(19) United States
(12) Patent Application Publication
(10) Pub. No.: US 2026/0207287 A1
(43) Pub. Date: Jul. 23, 2026

(54) SURGICAL DEVICE AND SYSTEM

(71) Applicant: Parallel Robotics LLC, Ann Arbor, MI (US)

(72) Inventor: Shorya Awtar, Ann Arbor, MI (US)

(73) Assignee: Parallel Robotics LLC, Ann Arbor, MI (US)

(21) Appl. No.: 19/467,163

(22) Filed: Feb. 2, 2026

Related U.S. Application Data
(63) Continuation of application No. PCT/US2024/040453, filed on Jul. 31, 2024.
(60) Provisional application No. 63/516,730, filed on Jul. 31, 2023.

(51) Int. Cl. A61B 34/00; A61B 17/00

(52) U.S. Cl. CPC A61B 34/71; A61B 17/00234; A61B 2017/00323; A61B 2017/00398; A61B 2017/0046

(57) ABSTRACT

A device for manipulating an object include a distal manipulator for engaging the object, a roll transmission that causes the distal manipulator to rotate, the roll transmission having a motor, and an articulation transmission that maintains the distal manipulator in the articulated orientations as the distal manipulator is rotated by the roll transmission. The distal manipulator is movable between a nominal orientation and articulated orientation and is rotatable in the articulated orientation with unlimited range of motion. The articulation transmission has an output member that is movable between another nominal orientation and other articulated orientations that correspond to the nominal orientation and the articulated orientation of the distal manipulator.

### CROSS-REFERENCE TO RELATED APPLICATION(S)

This application is a continuation of International Patent Application No. PCT/US2024/040453, filed Jul. 31, 2024, which claims priority to and benefit of U.S. Provisional Application No. 63/516,730, filed Jul. 31, 2023, the entire disclosures of which are incorporated by reference herein.

### TECHNICAL FIELD

This disclosure relates to a device for transmitting movement in three degrees of freedom, and in particular, for transmitting movement in three degrees of freedom to a surgical device.

### BACKGROUND

Various types of devices for manipulating objects, and more particularly for surgical instruments to perform laparoscopic movements and actions are known. It would be beneficial to provide a device which can provide an articulated rotation with unlimited range of motion.

### SUMMARY

Disclosed herein are implementations of a device for manipulating an object include a distal manipulator for engaging the object, a roll transmission that causes the distal manipulator to rotate, the roll transmission having a motor, and an articulation transmission that maintains the distal manipulator in the articulated orientations as the distal manipulator is rotated by the roll transmission. The distal manipulator is movable between a nominal orientation and articulated orientation and is rotatable in the articulated orientation with unlimited range of motion. The articulation transmission has an output member that is movable between another nominal orientation and other articulated orientations that correspond to the nominal orientation and the articulated orientation of the distal manipulator.

Also disclosed are implementations of a surgical device includes a distal manipulator for engaging an object, a handle assembly having a handle movable by a hand of a user between a second reference orientation and second articulated orientations and a roll input for receiving user input to rotate the distal manipulator in the articulated orientations, a roll transmission that causes the distal manipulator to rotate according to the user input, and an articulation transmission that maintains the distal manipulator in the articulated orientations as the distal manipulator is rotated by the roll transmission. The distal manipulator is movable between a reference orientation and articulated orientations and rotatable in the articulated orientations with unlimited range of motion. The articulation transmission includes an output member that is movable between a third reference orientation and third articulated orientations.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a schematic view of a device.

FIG. 2 is a simplified perspective view of an embodiment of the device of FIG. 1 in a first position and a first configuration.

FIG. 3 is a simplified perspective view of the embodiment of the device of FIG. 2 in a second position and the first configuration.

FIG. 4 is a simplified perspective view of the embodiment of the device of FIG. 2 in the first position and a second configuration.

FIG. 5A is another schematic view of the device of FIG. 1.

FIG. 5B is an upper, front, right perspective view of an embodiment of the device of FIGS. 1-5A.

FIG. 6 is a rear, right perspective view of the embodiment of the device of FIGS. 1-5A.

FIG. 7 is a schematic view of a first embodiment of an input transmission of the device of FIG. 5A.

FIG. 8 is a schematic view of a second embodiment of the input transmission of the device of FIG. 5A.

FIG. 9 is a side view of a distal manipulator of the device of FIGS. 1-5A in a first configuration.

FIG. 10 is a side view of the distal manipulator of FIG. 7 in the first configuration illustrating output cable lengths in dashed lines.

FIG. 11 is a top view of the distal manipulator of FIG. 7 in the first configuration illustrating output cable lengths in dashed lines.

FIG. 12 is a side view of the distal manipulator of FIG. 7 in the first configuration illustrating a device frame of reference and an output frame of reference.

FIG. 13 is a top view of the distal manipulator of FIG. 7 in the first configuration illustrating the device frame of reference and the output frame of reference.

FIG. 14 is a side view of the distal manipulator of FIG. 7 in the second configuration illustrating the device frame of reference and the output frame of reference.

FIG. 15 is a top view of the distal manipulator of FIG. 7 in the second configuration illustrating the device frame of reference and the output frame of reference.

FIG. 16 is a schematic view of the device of FIG. 5A further illustrating the input, intermediate, and output transmissions.

FIG. 17 is a simplified partial cross-sectional view of a chassis of the device of FIG. 5 and transfer devices thereof.

FIG. 18 is another simplified partial cross-sectional view of the chassis taken at 90 degrees relative to the view of FIG. 17.

FIG. 19 is a partial view perspective view of the chassis.

FIG. 20A is a perspective view of a surgical system having an exemplary device and a controller connected thereto.

FIG. 20B is a functional block diagram illustrating the function of an exemplary device and a controller connected thereto.

FIG. 21A is an upper, front, right perspective view of an exemplary device with an exemplary swashplate mechanism.

FIG. 21B is an upper, front, right perspective view of the exemplary device of FIG. 21A illustrating rotational axes.

FIG. 22A is a side view of the exemplary device of FIG. 21A.

FIG. 22B is an exemplary drive coupling of the device shown in FIG. 22A

FIG. 22C is side view of the exemplary device of FIG. 21A with a second drive coupling.

FIG. 23 is an upper, front, right perspective view of the exemplary device of FIG. 21A illustrating a first position of a roll rotation.

FIG. 24 is an upper, front, right perspective view of the exemplary device of FIG. 21A illustrating a second position of a roll rotation.

FIG. 25 is an upper, front, right perspective view of the exemplary device of FIG. 21A illustrating a third position of a roll rotation.

FIG. 26 is an upper, front, right perspective view of the exemplary device of FIG. 21A illustrating a fourth position of a roll rotation.

FIG. 27 is a simplified schematic view of the output frame of reference of the exemplary device.

FIG. 28A is an upper, front, right perspective view of an alternative configuration of the device.

FIG. 28B is an upper, front, right perspective view of an alternative configuration of the device.

FIG. 28C is an upper, front, right perspective view of an alternative configuration of the device.

FIG. 28D is a side view of an alternative configuration of the device.

FIG. 28E is a side view of an alternative configuration of the device.

FIG. 28F is a side view of an alternative configuration of the device.

FIG. 29A is a side view of an alternative configuration of the device.

FIG. 29B is a side view of an alternative configuration of the device.

FIG. 29C is a side view of an alternative configuration of the device.

FIG. 29D is an upper, front, right perspective view of an alternative configuration of the device.

FIG. 30A is a side view of an alternative configuration of the device.

FIG. 30B is a side view of an alternative configuration of the device.

FIG. 30C is a side view of an alternative configuration of the device.

FIG. 31A is a side view of an alternative configuration of the device.

FIG. 31B is a side view of an alternative configuration of the device.

FIG. 31C is a side view of an alternative configuration of the device.

FIG. 32 is a perspective view of a wristed apparatus that comprises the device.

FIG. 33 is a perspective view of the wristed apparatus of FIG. 32 is use by a user.

FIG. 34 is a perspective view of a handle of the wristed apparatus of FIG. 32.

FIG. 35 is a side view of a handle of the wristed apparatus of FIG. 32.

FIG. 36 is a side schematic view of a handle of the wristed apparatus of FIG. 32.

FIG. 37 is a side schematic view of a handle of the wristed apparatus of FIG. 32.

FIG. 38 is a perspective view of a feedback mechanism in the handle of the wristed apparatus of FIG. 32.

FIG. 39 is a perspective view of an input linkage assembly of the wristed apparatus of FIG. 32.

FIG. 40 is an exploded view of an input linkage assembly of the wristed apparatus of FIG. 32.

FIG. 41 is an exploded view of an input linkage assembly of the wristed apparatus of FIG. 32.

FIG. 42 is a schematic view of an input linkage assembly of the wristed apparatus of FIG. 32.

FIG. 43 is a collapsed and an exploded view of an input linkage assembly of the wristed apparatus of FIG. 32.

FIG. 44 is a schematic view of the swashbox assembly of the wristed apparatus of FIG. 32.

FIG. 45 is a schematic view of the pitch link subassembly of the wristed apparatus of FIG. 32.

FIG. 46 is a schematic view of the yaw link subassembly of the wristed apparatus of FIG. 32.

FIG. 47 is a schematic view of the articulation subassembly of the wristed apparatus of FIG. 32.

FIG. 48 is a schematic view of the articulation subassembly of the wristed apparatus of FIG. 32.

FIG. 49 is a schematic view of the articulation subassembly of the wristed apparatus of FIG. 32.

FIG. 50 is a schematic view of the swashbox subassembly of the wristed apparatus of FIG. 32.

FIG. 51 is an exploded schematic view of the articulation subassembly of the wristed apparatus of FIG. 32.

FIG. 52 is a schematic view of the swashbox subassembly of FIG. 50.

FIG. 53 is a schematic view of the swashbox subassembly of FIG. 50.

FIG. 54 is a schematic view of the swashbox subassembly of FIG. 50.

FIG. 55 is a schematic view of the swashbox subassembly of FIG. 50.

FIG. 56 is a schematic view of the swashbox subassembly of FIG. 50.

FIG. 57 is a schematic view of the swashbox subassembly of FIG. 50.

FIG. 58 is a schematic view of the swashbox subassembly of FIG. 50.

FIG. 59 is a schematic view of the swashbox subassembly of FIG. 50.

FIG. 60 is a schematic view of the roll transmission subassembly of the wristed apparatus of FIG. 32.

FIG. 61 is an exploded schematic view of the roll transmission subassembly of the wristed apparatus of FIG. 32.

FIG. 62 is a schematic view of a tool shaft subassembly of the wristed apparatus of FIG. 32.

FIG. 63 is a schematic view of a tool shaft subassembly of the wristed apparatus of FIG. 32.

FIG. 64 is a schematic view of the swashbox subassembly of the wristed apparatus of FIG. 32.

FIG. 65 is a schematic view of an alternative handle assembly.

FIG. 66 is a schematic view of an alternative handle assembly.

FIG. 67 is a schematic view of an alternative handle assembly.

FIG. 68 is a schematic view of an alternative handle assembly.

FIG. 69 is a schematic view of an alternative handle assembly.

### DETAILED DESCRIPTION

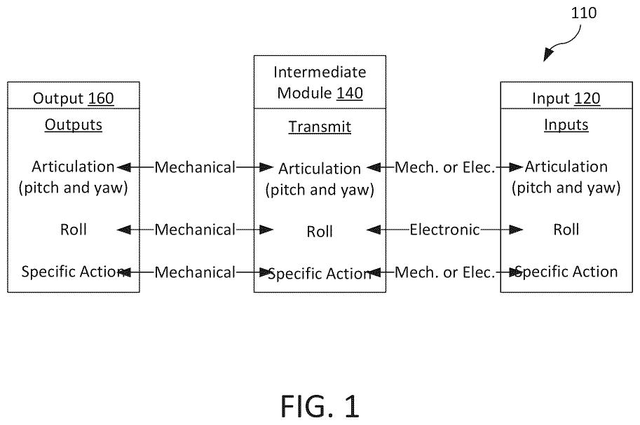

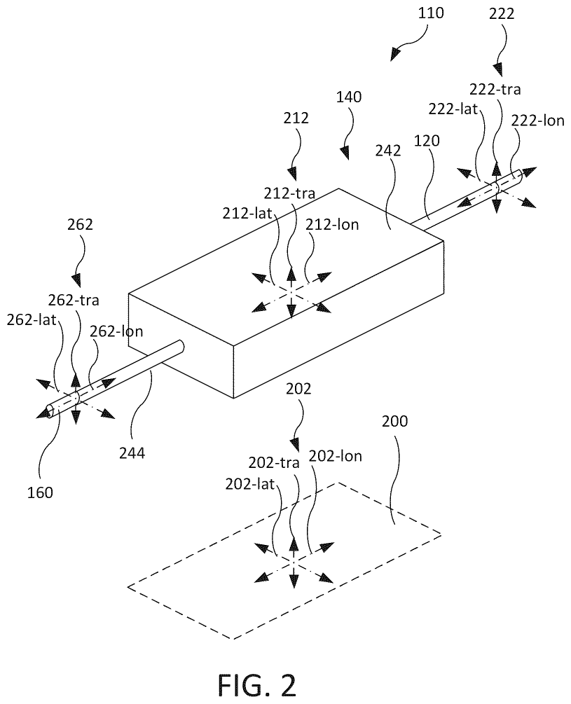

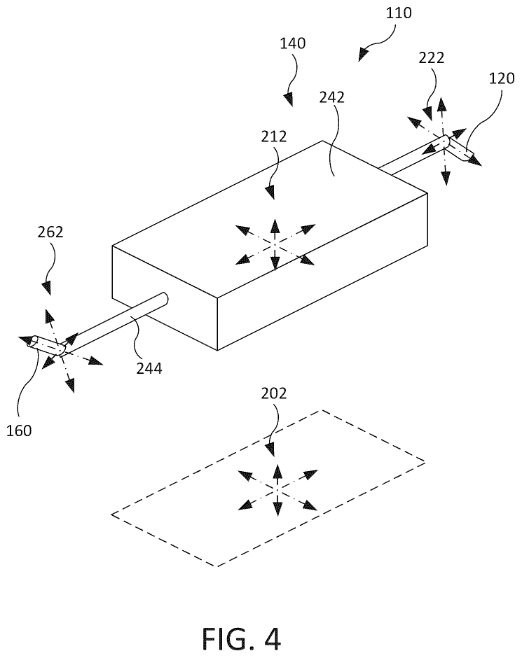

Referring to FIGS. 1-4, a device 110 is configured for a user to provide wrist-like movement in three degrees of freedom through a device with a swashplate mechanism. In particular, the device 110 may be configured for a user, such as a surgeon, to perform minimally invasive surgical procedures on a patient 200, such as laparoscopy. The patient 200 is schematically represented by a portion of a plane in FIGS. 2-4.

The device 110 generally includes an input (e.g. input end) 120, an intermediate module (e.g. intermediate portion) 140, and an output (e.g. output end) 160. The input end 120 receives user inputs from a user. The user inputs are typically motion inputs (e.g. rotation or translation or a combination thereof) and/or load inputs (e.g. torque or force or a combination thereof). The output end 160 provides physical outputs. The intermediate portion 140 converts, transmits, or otherwise transfers the user inputs received at the input end 120 into the physical outputs provided at the output end 160.

As illustrated in FIG. 1, the user inputs may include articulation (e.g. pitch and yaw rotation), roll rotation, and specific actions (e.g. tool inputs), which may be transferred from the input end 120 to the intermediate portion 140 mechanically or electronically. The physical outputs of the output end 160 may also include articulation (e.g. pitch and yaw rotation), roll rotation, and tool outputs, which are transferred from the intermediate portion 140 to the output end 160. Roll rotation may be simply referred to as “roll”. As used here, the terms “convert”, “transmit”, or “transfer” generally refer to the transmission by the intermediate portion 140 of user inputs received at the input end 120 into physical outputs at the output end 160.

The physical outputs provided by the output end 160 include movement outputs and tool outputs. The movement outputs include articulation and roll, of the output end 160 relative to the patient 200, the device 110, or both. Movement outputs may include one or more translations of the output end 160 relative to the patient 200. The tool output includes operation of the output end 160 to operate a tool thereof. As examples, the input end 120 may include a handle that is movable relative to the intermediate portion 140 to receive the user inputs to the surgical device 110, while the output end 160 may include a tool, such as a distal manipulator for engaging an object, that is movable relative to the intermediate portion 140 and may include jaws that are operated by being opened and closed (e.g., to grasp and release an object, such as a needle for suturing). The distal manipulator may itself be a tool (e.g., an end effector) or include another tool coupled thereto that is the object (e.g., another type of end effector or non-contact tool, such as a laser or sensor).

Referring to FIGS. 2-4, as referenced above, the user inputs and the physical outputs include movements relative to the patient 200, the device 110, or both which may be considered to form a patient frame of reference 202 and a device frame of reference 212, respectively. It is noted that the patient 200 may move in space, such as an operating room, such that the patient frame of reference 202 may move relative to space (e.g., an absolute or true ground frame of reference).

The patient frame of reference 202 may be defined to include a patient longitudinal axis 202-lon extending longitudinally through the patient 200 (e.g., from head to toe), patient lateral axis 202-lat extending laterally through the patient 200 (e.g., from left to right), and a patient transverse axis 202-tra extending through the patient (e.g., from front to back), which substantially perpendicular to each other and are fixed relative to the patient 200. The different axes of the patient frame of reference 202 may be defined relative to the patient in any other suitable manner.

The device frame of reference 212 may be defined to include a device longitudinal axis 212-lon, a device lateral axis 212-lat, and a device transverse axis 212-tra that are substantially perpendicular to each other and fixed relative to the intermediate portion 140 or a fixed reference portion thereof, such as a chassis 242 or other stationary portion (relative to the device). Aspects of the input end 120 and the output end 160 move relative to the device frame of reference 212. The device longitudinal axis 212-long may generally correspond to a direction in which the output end 160 is inserted into the patient 200. The device longitudinal axis 212-lon may extend through the output end 160 when the output end 160 is in a reference or nominal (i.e., not articulated) orientation relative to the device frame of reference 212. The output end 160 may be coupled to the chassis 242 via member 244 that extends from the intermediate portion 140. In the case of the member 244 being a straight shaft, the device longitudinal axis 212-lon may also extend through the member 244, such as being coaxial therewith, and the chassis 242 of the intermediate portion 140.

The output end 160 may also be considered to have an output frame of reference 262. The output frame of reference 262 generally includes an output longitudinal axis 262-lon, a output lateral axis 262-lat, and a output transverse axis 262-tra that are substantially perpendicular to each other and fixed relative to a portion of the output end 160, such as a base thereof. As the output end 160 is moved relative to the reference portion of the device 110, the output frame of reference 262 moves relative to the device frame of reference 212.

The input end 120 may also be considered to have an input frame of reference 222. The input frame of reference 222 generally includes an input longitudinal axis 222-lon, an input lateral axis 222-lat, and an input transverse axis 222-tra that are substantially perpendicular to each other and fixed relative to a portion of the input end 120, such as a handle thereof. As the input end 120 is moved relative to the reference portion of the device 110, the input frame of reference 222 moves relative to the device frame of reference 212.

As referenced above, the physical outputs of the output end 160 include movement of the output end 160 relative to the patient frame of reference 202, or the device frame of reference 212, or both. Physical outputs can include translations, articulation, and roll rotation.

The translational outputs of the output end 160 include translation of the output end 160 with respect to the patient frame of reference 202. Translational movement of the output end 160 may be achieved by moving the entire surgical device 110 relative to the patient 200, approximately along the device longitudinal axis 212-long, the device lateral axis 212-lat, the device transverse axis 212-tra, or any combination thereof. For example, the output end 160 may be inserted into the patient 200 through a port placed in an incision on the patient's body about which the device 110 pivots, such that the translational movement of the output end 160 may be achieved by moving the intermediate portion 140 of the surgical device 110 relative to the patient 200 (i.e., insertion and retraction, lateral movement to the left and right, and transverse movement up and down).

The articulation output includes articulation of the output end 160 relative to one or two respective axes, such as the device lateral axis 212-lat, the device transverse axis 212-tra, or both. The term articulation is considered to include bending, rotating, or pivoting relative to an axis, for example, generally about an axis of a frame of reference or line parallel thereto.

Articulation of the output end 160 may be achieved by rotating, bending, or pivoting the output end 160 relative to the device lateral axis 212-lat, the device transverse axis 212-tra, or both. Articulation relative to the device lateral axis 212-lat may be referred to as pitch rotation (or simply pitch), while articulation relative to the device transverse axis 212-tra may be referred to as yaw rotation (or simply yaw).

As referenced above, the output end 160 is in the reference (or nominal) orientation when the output longitudinal axis 262-lon is coaxial or otherwise parallel with the device longitudinal axis 212-lon. The output end 160 is considered to be in an articulated orientation when the output end 160 is at a non-zero angle relative to the device lateral axis 212-lat, the device transverse axis 212-tra, or both, such that the output longitudinal axis 262-lon is not coaxial or otherwise parallel with the device longitudinal axis 212-lon. From the reference orientation, the output end 160 may articulate through ranges of motion about to the device lateral axis 212-lat, the device transverse axis 212-tra, or both, which may be referred to as a pitch range of motion and a yaw range of motion, respectively, and which may be substantially symmetric relative to the reference orientation. The terms “reference orientation” and “nominal orientation” may be used interchangeably.

The roll output includes rotation of the output end 160 about the device longitudinal axis 212-lon when the output end 160 is in the reference orientation. When the output end 160 is articulated about the device lateral axis 212-lat, the device transverse axis 212-tra, or both and while maintaining this articulated orientation, then rotation of the output end 160 about the output longitudinal axis 262-lon. As roll of the output end 160 occurs, the output lateral axis 262-lat and the output transverse axis 262-tra rotate about the output longitudinal axis 262-lon that itself may remain stationary in the articulated orientation relative to the device longitudinal axis 212-lon.

The tool output of the output end 160 may, for example, include opening and closing of jaws that form a distal manipulator of the output end 160.

The device 110 is configured for different physical outputs of the output end 160 to be performed independent of each other, such that performance of different physical outputs neither require nor cause operation of other ones of the physical outputs. As listed in Table 1 below, the translation outputs, the articulation output, the roll output (including articulated roll), and the tool output may all be performed independent of each other. It should be noted that the physical outputs of the output end 160 may be independent of each other, while any devices, structures, mechanisms, and intervening actions that cause the physical outputs may still be dependent on each other (e.g., the operation or movement of one such device, structure, mechanisms action may require or cause operation or movement of another). For example, articulated roll may be achieved by operating those mechanisms that would cause rotation of the member 244 and the output end 160 relative to the device longitudinal axis 212-lon, while simultaneously operating those mechanisms that cause articulation of the output end 160 to maintain the output longitudinal axis 262-lon in an articulated orientation as opposed to tracing a generally conical shape.

TABLE 1
Independent Physical Outputs

| Independent Mechanical Outputs | Frame of Reference |
|---|---|
| Longitudinal translation | Patient |
| Lateral translation | Patient |
| Transverse translation | Patient |
| Pitch | Device |
| Yaw | Device |
| Roll | Device |
| Roll in an articulated configuration | Output |
| Tool | Output |

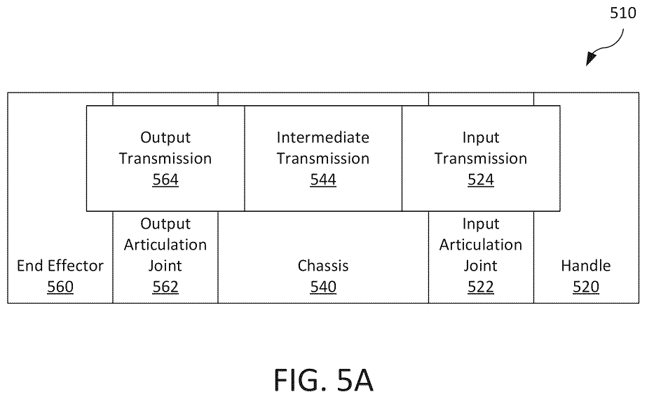

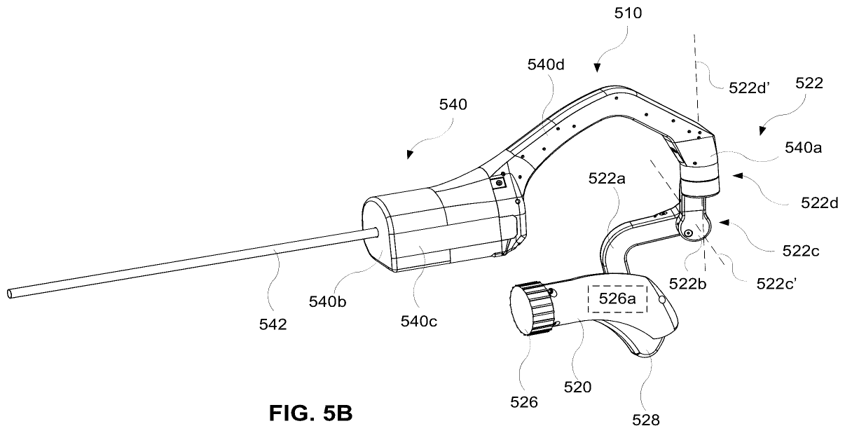

The input end 120 is configured to receive user inputs from the user, which the intermediate portion 140 receives, converts, transmits, or otherwise transfers these to physical outputs provided at the output end 160. Transfer and conversion of the user inputs into the physical outputs may be performed cooperatively by the input end 120, the intermediate portion 140, and the output end 160 using any suitable combinations of mechanisms and devices (e.g., cables, pulleys, linkages, other mechanisms, sensors, transducers, motors, and other electronic devices), which may be referred to as transmissions and are discussed with reference to FIG. 5A and onward.

The input end 120 may be configured to receive user inputs for articulation, such as pitch, yaw, or both, and may also be configured to receive user inputs for roll and for operating the tool. User inputs to control articulation may be generally referred to as articulation inputs (including pitch input, or yaw input, or both). User inputs to control roll may generally be referred to as roll input. User inputs for operating a tool may generally be referred to as tool inputs.

To receive the user inputs for pitch, yaw, or both, the input end 120, which may include a handle, may be articulated about the input lateral axis 222-lat, the input transverse axis 222-tra, or both and, thereby, relative to the intermediate portion 140. Articulation about the input lateral axis 222-lat may be referred to as a pitch input, while articulation about the input transverse axis 222-tra may be referred to as yaw input. As the input end 120 is articulated relative to the device frame of reference 212, the input frame of reference 222 moves relative thereto.

The pitch input, the yaw input, or both may be transmitted entirely mechanically from the input end 120 through the intermediate portion 140 and into the pitch output, the yaw output, or both provided at the output end 160. In this case, the pitch input, the yaw input, or both may be received mechanically by the input end 120 and mechanically transferred from the input end 120 to the intermediate portion 140. The intermediate portion 140 in turn mechanically converts the pitch input, the yaw input, or both into the pitch output, the yaw output, or both that are mechanically transferred from intermediate portion 140 to the output end 160 to be provided thereby.

Alternatively, the pitch input, the yaw input, or both may be transmitted by a combination of mechanically and electronically (such combination commonly referred to as “mechatronically”) from the input end 120 through the intermediate portion 140 and into the pitch output, the yaw output, or both provided by the output end 160. In one example, the pitch input, the yaw input, or both may be received mechanically by the input end 120 converted to electronic signals using transducers such as sensors. For example, a portion of the input end 120, such as the handle, may be articulated about the input lateral axis 222-lat, the input transverse axis 222-tra, or both, which is measured by sensors according to which electronic signals, such as a pitch signal, a yaw signal, or both are captured and transferred to a microcontroller. The microcontroller may command electromechanical actuators such as motor located in the intermediate portion 140. Typically, such a command from a microcontroller to a motor is sent via a motor driver. The motor in turn provides motions that are mechanically transferred from the intermediate portion 140 into the pitch output, the yaw output, or both provided by the output end 160.

The input end 120 may also be configured to receive user inputs for roll. To receive inputs for roll, a portion of the input 120, such as a knob or a dial (for example, referred to as roll dial), may be rotated by the user, for example, about the input longitudinal axis 222-lon. The roll input may be transmitted by a combination of mechanically and electronically (i.e. mechatronically) from the input end 120 through the intermediate portion 140 and into the articulated roll output provided by the output end 160. In one example, the roll input is received mechanically by the input end 120 and electronically transferred to a microcontroller. For example, the rotation of the input end 120, is measured and transferred to the microcontroller. The microcontroller may command an electromechanical actuator such as a roll motor located in the intermediate portion 140. The roll motor in the intermediate portion 140 in turn produces the roll rotation that is mechanically transferred from the intermediate portion 140 to the output end 160 to be provided thereby.

Alternatively, the roll input may be transmitted entirely mechanically from the input end 120 through the intermediate portion 140 and into the articulated roll output provided by the output end 120. In this case, roll input is received mechanically by the input end 120 and mechanically transferred from the input end 120 to the intermediate portion 140. The intermediate portion 140 in turn mechanically transforms or converts the roll input into the roll output that is mechanically transferred from intermediate portion 140 to the output end 160 to be provided thereby.

To receive inputs for operating the tool, the input end 120 may include a tool input portion that is engaged by the user, such as a lever or trigger or button or pressure pad that is pulled or pressed or pushed or otherwise engaged or actuated by the user. Engagement of the tool input portion by the user may be referred to as a tool input. The tool input may be transmitted entirely mechanically from the input end 120 through the intermediate portion 140 and into the tool output provided by the output end 160. In this case, tool input is received mechanically by the input end 120 and mechanically transferred from the input end 120 to the intermediate portion 140. The intermediate portion 140 in turn mechanically transmits or converts the tool input into the tool output that is mechanically transferred from intermediate portion 140 to the output end 160 to be provided thereby.

Alternatively, the tool input may be transmitted by a combination of mechanically and electronically (i.e. mechatronically) from the input end 120 through the intermediate portion 140 and into the tool output provided by the output end 160. In one example, the tool input is received mechanically by the input end 120, such as the lever, and electronically transferred to a microcrontroller. For example, movement of the lever of the input end 120 may be measured by one or more sensors or transducers according to which electronic signals, such as a tool signal, are transferred to the microcontroller. The microcontroller in turn commands an electromechanical actuator (such as a motor) located in the intermediate portion 140 or elsewhere to produce a tool output that is mechanically transferred from the intermediate portion 140 to the output end 160 to be provided thereby.

The device 110 is configured for different user inputs to the input end 120 to be received independent of each other, such that receipt of different user inputs neither require nor cause receipt nor cause interference of another user input. As noted above, the user inputs may be received mechanically at the input end 120, and may be respectively transmitted mechanically or mechatronically from the input end 120 to the intermediate portion 140. Different possible combinations of mechanical and electronic (or mechatronic) transmissions are outlined in Table 2 below. The terms “mechatronic” and “electronic” in the context of motion transmission are used interchangeably here. Similarly, “mechatronically” and “electronically” are used interchangeably in the context of motion transmission.

TABLE 2
Combinations of Mechanical and Electronic
Movement Transmission

| User Input | Pitch | Yaw | Roll |
|---|---|---|---|
| Embodiment 1 | Mechanical | Mechanical | Electronic |
| Embodiment 2 | Mechanical | Electronic | Electronic |
| Embodiment 3 | Electronic | Mechanical | Electronic |
| Embodiment 4 | Electronic | Electronic | Electronic |
| Embodiment 5 | Mechanical | Mechanical | Mechanical |
| Embodiment 6 | Mechanical | Electronic | Mechanical |
| Embodiment 7 | Electronic | Mechanical | Mechanical |
| Embodiment 8 | Electronic | Electronic | Mechanical |

Referring to FIGS. 5A-11, a device 510 is an embodiment of a surgical device 510 or wristed apparatus. The device 510 generally includes a handle assembly 520, an input articulation joint 522, a chassis 540 (or frame), a shaft 542, a distal manipulator 560, and an output articulation joint 562. The chassis 540 is structurally coupled to the handle 520 at a first side 540a thereof via the input articulation joint 522 and to the shaft 542 at a second side 540b thereof. The distal manipulator 560 is structurally coupled to the shaft 542 (e.g., being pivotably coupled to allow pivoting therebetween and to transfer toque therebetween) via the output articulation joint 562.

The handle assembly 520 forms the input end 120 and is associated with the input frame of reference 222. The input articulation joint 522, the chassis 540, the shaft 542, and the output articulation joint 562 generally form the intermediate portion 140 and are associated with the device frame of reference 212, for example, with the device longitudinal axis 212-lon being coaxial with the shaft 542. The distal manipulator 560 forms the output end 160 and is associated with the output frame of reference 262. The handle assembly 520 is movable between a nominal orientation, which corresponds to the nominal orientation of the distal manipulator 560 and the output member 1752 discussed below, and one or more articulated orientations, which correspond to articulated orientations of the distal manipulator 560 and the output member 1752 discussed below. Thus, movement of the handle assembly 520 in pitch and yaw directions (e.g., relative to the chassis 540 or the device frame of reference 212) causes the distal manipulator 560, the output member 1752, or both to move in pitch and yaw directions (e.g., also relative to the chassis 540 or the device frame of reference 212).

The device 510 further includes a series of transmissions by which the handle 520 is functionally coupled to the distal manipulator 560. Each of the transmissions includes a series of intervening systems, mechanisms, and devices (e.g., cables, pulleys, linkages, other mechanisms, sensors, transducers, motors, other electronic devices, and systems thereof) to transfer and convert the user inputs (e.g., pitch, yaw, and roll) into the physical outputs of the distal manipulator 560 (e.g., pitch, yaw, and roll). For example, the surgical device 510 may be considered to include an input transmission 524, an intermediate transmission 544, and an output transmission 564, which cooperatively function to transfer and convert the user inputs (e.g., pitch and yaw) into physical outputs (e.g., pitch and yaw) of the distal manipulator 560. The input transmission 524 transfers the user inputs from the handle 520 to the intermediate transmission 544. The input transmission 524 generally extends from the handle 520 and via the input articulation joint 522 to the intermediate transmission 544. The intermediate transmission 544 converts and transmits the user inputs into the physical outputs. The intermediate transmission 544 is generally contained by or otherwise coupled to the chassis 540. The output transmission 564 transfers the physical outputs from the intermediate transmission 544 to the distal manipulator 560 to be provided thereby. The output transmission 564 extends from the intermediate transmission 544 to the output articulation joint 562 and the distal manipulator 560. Each of the different transmissions may be considered to include multiple transmissions by which the user inputs and physical outputs are transmitted and converted for different functions, such as articulation, roll, or tool. Some aspects of the input transmission 524 and the intermediate transmission 544 may be performed electronically (or mechatronically). Further aspects of the transmissions are discussed in further detail below.

The handle 520, which may also be referred to as a handle body, is movably coupled to the chassis 540 (e.g. the frame) to receive articulation inputs from the user for controlling articulation of the distal manipulator 560, such as pitch, yaw, or both. The handle 520 includes a first end that is movably coupled to the first side 540a of the chassis 540. For example, the first end of the handle 520 may be coupled to the chassis 540 with the input articulation joint 522 that includes first link 522a and a second link 522b. The first end of the handle 520 is fixedly coupled to the first link 522a that is rotatably coupled to the second link 522b at a first pivot joint 522c. The second link 522b is rotatably coupled to the chassis 540 at a second pivot joint 522d. The first link 522a and the second link 522b are generally rigid structures, which are generally configured to not bend or otherwise deflect when receiving the user inputs for controlling articulation of the distal manipulator 560.

The first pivot joint 522c and the second pivot joint 522d are each configured to permit rotation of the handle 520 about only one respective axis relative to the chassis 540, while constraining movement of the handle 520 in and about other axes. For example, the pivot joints 522c, 522d may be configured as pin/pivot/revolute joints that include a post rigidly coupled to one structure (e.g., the first link 522a, the second link 522b, or the chassis 540) and a receptacle formed by the other structure (e.g., the other adjacent one of the first link 522a, the second link 522b, or the chassis 540), the post being received and rotatable within the receptacle (e.g., each having circular cross sectional shapes or having a bearing interface).

The first pivot joint 522c forms a first pivot axis 522c′ about which the first link 522a and, thereby, the handle 520 rotate relative to the second link 522b and the chassis 540. The first pivot axis 522c′ may, for example, form or be parallel with the input lateral axis 222-lat discussed previously. When there is no yaw input (i.e. yaw input is zero), then this axis also coincides with or is parallel to the device lateral axis 212-lat discussed previously. Rotation of the handle 520 about the first pivot axis 522c′ may, for example, be a pitch input by which the user controls pitch output of the distal manipulator 560. The first link 522a may be referred to as a pitch link, the first pivot joint 522c may be referred to as an input pitch joint (or simply pitch joint), and the first pivot axis 522c′ may be referred to as an input pitch axis.

The second link 522b is coupled to the first side 540a of the chassis 540 at a second pivot joint 522d. The second pivot joint 522d forms a second pivot axis 522d′ about which the second link 522b and, thereby, the first link 522a and the handle 520, rotate relative to the chassis 540. The pivot axis 522d′ may, for example, form or be parallel with the device transverse axis 212-tra and/or the input transverse axis 222-tra, or both discussed previously. Rotation of the handle 520 about the second pivot axis 522d′ may, for example, be a yaw input by which the user controls yaw output of the distal manipulator 560. The second link 522b may be referred to as a yaw link, the second pivot joint 522d may be referred to as an input yaw joint (or simply yaw joint), and the second pivot axis 522d′ may be referred to as an input yaw axis.

It is noted that while the first and second pivot axes 522c′, 522d′ are described and illustrated as corresponding to the device lateral axis 212-lat, the device transverse axis 212-tra, the pitch input, and the yaw input, the first and second pivot axes 522c′, 522d′ may be arranged in other manners, for example, being rotationally offset (e.g., 45 degrees) from the device lateral axis 212-lat and the device transverse axis 212-tra. In this manner, the handle 520 may still be pivoted about the device lateral axis 212-lat and the device transverse axis 212-tra to provide pitch and yaw inputs, respectively; however, such pivoting results in different combinations about the first and second pivot 522c′, 522d′ that provide each of the pitch input and the yaw input. For example, to change pitch of the distal manipulator 560, the handle 520 is pivoted about the device lateral axis 212-lat, which results in pivoting of the handle about both the first and second pivot joints 522c, 522d and the first and second pivot axes 522c′, 522d′ defined thereby.

As illustrated, the handle 520 is configured to be grasped by the hand of a user. The handle 520 may be generally elongated and extend forward from a proximal end near the user that is coupled to the first link 522a to a distal end near the patient that is free (i.e., not coupled to any structure). The pivot axes 522c′, 522d′ may generally coincide (e.g., intersect) the wrist of the user, such that the user may pivot their hand upward and downward about their wrist to pivot the handle 520 to provide the pitch input and leftward and rightward to provide the yaw input. For example, the first link 522a extends from the handle 520 laterally outward in the general direction of the device lateral axis 212-lat, upward in the general direction of the device transverse axis 212-tra, and rearward toward the user in the general direction of the device longitudinal axis 212-lon to the first pivot joint 522c. The second link 522b extends from the first pivot joint 522c upward in the general direction of the device transverse axis 212-tra, and laterally inward in the general direction of the device lateral axis 212-lat to the second pivot joint 522d.

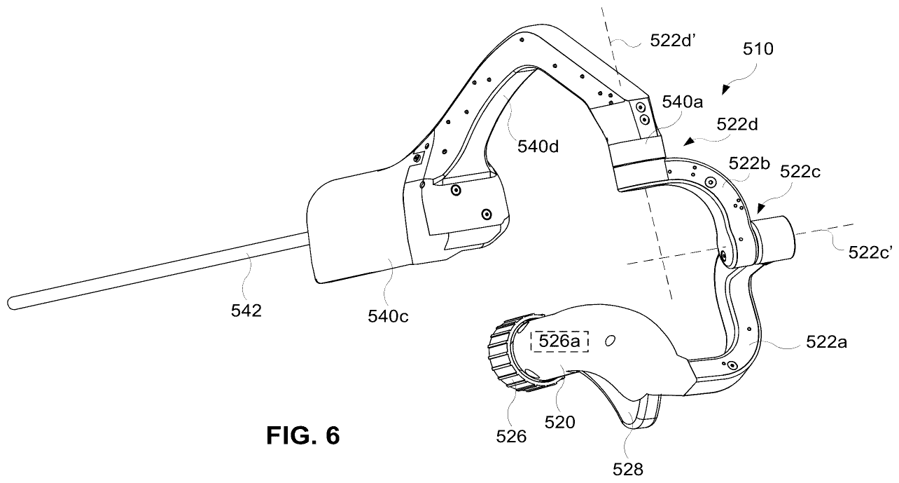

As shown in FIGS. 5B and 6, the chassis 540 (i.e., frame) includes a housing 540c (frame box) that contains other components of the device 510, such as various aspects of the input transmission 524, the intermediate transmission 544, and the output transmission 564. The chassis 540 further includes an arm 540d (i.e. frame arch) that extends rearward from the housing 540c toward the handle 520 to form the second side 540a of the chassis 540 that is coupled to the handle 520. For example, the arm 540d may extend upward and rearward over the handle 520 and terminate at the first side 540a of the chassis 540 at which the second pivot joint 522d is positioned above the wrist of the user. Aspects of the transmission 524 may extend through the first side 540a, the arm 540d, and the housing 540c of the chassis 540.

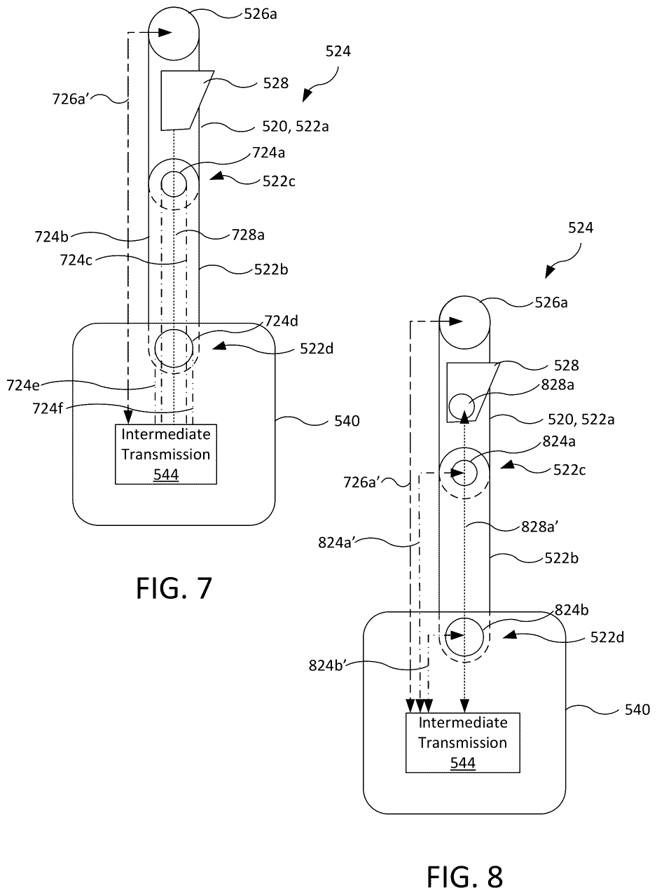

Referring to FIG. 7, rotation of the handle 520 about the first pivot axis 522c′, the second axis 522d′, or both, which may respectively form the pitch input, the yaw input, or both, is transferred to the chassis 540 mechanically. For example, the device 510 includes the input transmission 524 that transfers rotation of the handle 520 about the first pivot axis 522c′ and the second pivot axis 522d′, which may alone or in combination provide the pitch input and the yaw input, to the intermediate transmission 544 inside the chassis 540. The input transmission 524 includes first and second cable lengths 724b, 724c that extend from a first pulley 724a at the first pivot joint 522c through the second link 522b, through the second pivot joint 522d and into the chassis 540, through the arm 540d and into the housing 540c. As the handle 520 is rotated about the first pivot axis 522c′ in positive and negative directions, such as upward and downward to control pitch of the distal manipulator 560, the first and second cable lengths 724b, 724c, respectively, are pulled. The intermediate transmission 544 inside the housing 540c of the chassis 540, which is discussed in further detail below, in turn further receives, converts, and transmits movement of the first and second cable lengths 724b, 724c to the output transmission 564 that causes the distal manipulator 560 to provide the pitch output, the yaw output, or both. A key feature of this cable transmission is that as the first and second cable lengths 724b, 724c that extend from a first pulley 724a at the first pivot joint 522c are routed through the through the second pivot joint 522d, the rotation of the handle 520 about the second pivot axis 522c′ (i.e. rotation of the second pivot joint 522d) should not influence the cable lengths 724b and 724c. In other words, the input pitch transmission should not be influenced by the yaw rotation. To accomplish this decoupling, several strategies can be employed. One strategy is to route the cables 724b and 724c as close as possible to the rotation axis 522d′ of the second pivot joint 522d. Another strategy is to use a Bowden cable arrangement wherein the cables 724b and 724c are routed via flexible sheaths, as they traverse the second pivot joint 522d.

Rotation of the handle 520 about the second pivot axis 522c′ may similarly be transmitted from the second pivot joint 522d by the input transmission 524. The input transmission 524 further includes another set of first and second cable lengths 724e, 724f that extend from a second pulley 724d at the second pivot joint 522d into the chassis 540, such as through the arm 540d and into the housing 540c, to the intermediate transmission 544. As the handle 520 is rotated about the second pivot axis 522d′ in positive and negative directions, such as leftward and rightward to control yaw of the distal manipulator 560, the first length and second cable lengths 724e, 724f, respectively, are pulled. The intermediate transmission 554 in turn further receives, converts, and transmits movement of the first and second cable lengths 724e, 724f to cause movement output of the distal manipulator 560, such as by yawing the distal manipulator 560 to the output transmission 564 that causes the distal manipulator 560 to provide the pitch output, the yaw output or both.

The transmissions 524, 544, 564 may be cooperatively configured for the magnitude of the user input to the handle 520 (e.g., angle of pitch, yaw, or both) to generally correspond to the magnitude of physical output of the distal manipulator 560 (e.g., angle of pitch, yaw, or both) by being substantially equal (e.g., within 20%, 10%, or less of each other) or to magnify or demagnify the user input to physical output transmission ratio (e.g., ratio of 4:1, 3:1, 2:1, or less, or 1.5:1, 2:1, 3:1, 4:1 or more).

The input transmission 524, while described as including the pulleys 724a, 724d, and the cable lengths 724b, 724c, 724e, 724f may be implemented in other manners suitable for mechanically transferring movement of the handle 520 to the intermediate transmission 544, for example, using suitable combinations of cables, chains, belts, gears, and/or linkages.

The handle 520 may further include an articulated roll input 526, which is configured to receive user inputs for controlling roll of the distal manipulator 560. The articulated roll input 526 may, as shown, be a knob or dial (e.g. roll dial) that is rotatable relative to the handle 520 about axis 222-lon. For example, as shown, the second end of the handle 520 is a distal end that is arranged away from the user and to which the articulated roll input 526 is rotatably coupled. Alternatively, the rol input 526 may be physically separate from the device 510.

The handle 520 further includes a roll input sensor 526a (e.g. roll dial encoder), which is depicted schematically in FIGS. 7-8, such as a rotary encoder, that measures rotation of the articulated roll input 526 relative to the handle 520. The roll input sensor 526a sends electronic signals 726a′, which may be referred to as roll input signals, according to rotation of the roll input 526 relative to the handle 520. The intermediate transmission 544, such as a controller (or controller box) thereof, receives the roll input signals 726a′, and causes roll of the distal manipulator 560 according thereto. The articulated roll input 526 may have an unlimited rotational range of motion relative to the handle 520 in both clockwise and counter clockwise directions, such that the roll input 526 may be continuously rotate to, thereby, cause continuous rotation of the distal manipulator 560 in clockwise and counterclockwise directions, respectively.

The transmissions 524, 544, 564 may be cooperatively configured for the magnitude of the roll input to generally be equal to the magnitude of roll (including articulated roll) of the distal manipulator 560 with their respective angular positions relative to the chassis 540, which is generally preferable for a user. But the transmissions 524, 544, and/or 564 may also be configured to magnify or scale-down the roll input to roll output ratio (e.g., ratio of 4:1, 3:1, 2:1, or less, or 1.5:1, 2:1, 3:1, 4:1 or more between changes to the input and the output). With the magnitudes be generally equal, the roll input 526 may provide a visual and/or physical indicator to the user of the roll position of the distal manipulator 560 within the body of a patient.

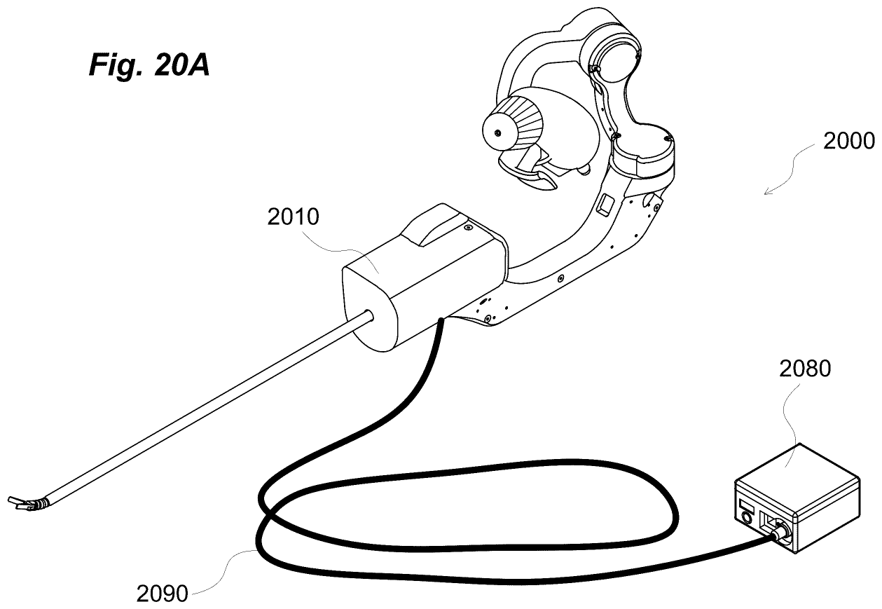

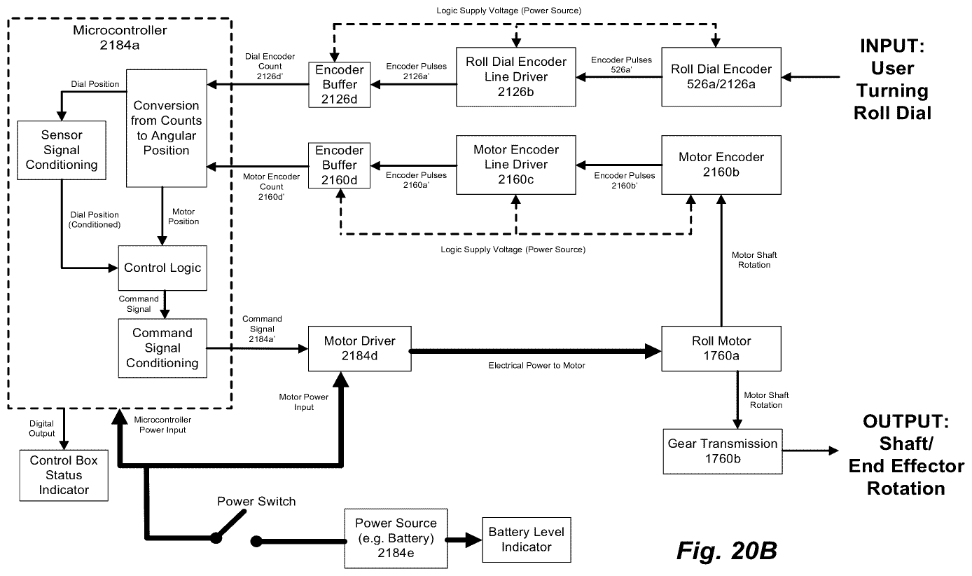

Referring to FIG. 20A, an embodiment of the device 110 is illustrated and includes various electromechanical components (such as motor, which may be referred to as a roll motor, to drive the roll rotation, and a roll input 526, such as a roll dial 526 and roll dial encoder 526a that measures a roll input angle input to the roll dial 526 by the user), as well as other electronic components (such as microcontroller, motor driver, encoder line driver, encoder buffer board, etc.), as described throughout this document.

In FIG. 20A, a system 2000 generally includes a device 2010, which is an embodiment of the device 110, 510, an electronics unit 2080, and a cable 2090 that connects the device 2010 to the electronics unit 2080 for power and signal transfer therebetween. The device 2010 is generally configured as described previously for the devices 110, 510, for example, that is an embodiment of the device 110, 510, a control box 2080, and a control cable that connects the device 110 with the control box 2080 for transferring power and signals therebetween. The device 2010 is generally configured as the devices 110, 510 described previously by including the input end 120, the intermediate portion 140, and the output end 160. The input end 120 may be configured as described for the device 510 by including the handle 520 with the articulation joint 522, the input transmission 524, the roll input 526, the tool input 528, and their associated systems and components, which may be adapted for the different configuration shown in FIG. 20A (e.g., with the various links 522a, 522b being arranged to be below the hand of the user instead of above as shown with the device 510). The input articulation joint 522 may include the first link 522a (e.g., a pitch link), the first joint 522b (e.g., the pitch joint), the second link 522b (e.g., the yaw link), the second joint 522d (e.g., the yaw joint), and other components and sub-assemblies as described herein and as may be appropriate. The roll input 526 may be configured as the roll dial 526 and a roll dial encoder 526a that senses rotation therewith, along a roll dial encoder line driver 2126b and other components and sub-assemblies as may be appropriate.

The intermediate portion 140 may be configured as described for the device 510 by including the chassis 540, which includes the housing 540c and the arm 540d (e.g., the frame arch) and may include and/or contain the intermediate transmission 544. The intermediate transmission includes the articulation transmission 1646 (e.g., the swash plate mechanism 1746) and the roll transmission 1648. The roll transmission 1648 includes the roll motor 1760a, a motor encoder 2160b, a motor encoder line driver 2160c and various other mechanical and electromechanical components associated therewith and contained in the chassis 540 (e.g., within the housing 540c).

The output end 160 of the device 2010 further includes the shaft 542, the output articulation joint 562, the distal manipulator 560 that may include jaws, a mechanism for opening and closing the jaws, and other components and sub-assemblies as described herein and as may be appropriate.

The electronics unit 2080 includes a housing (shown; not labeled) and various electronic components contained therein, including a microcontroller 2184a, an encoder buffer board 2126b, a power supply 2184e (e.g., a battery), a motor driver 2184d, and other components and systems as described herein and as may be appropriate.

Referring to FIG. 20B, which depicts a mechatronics system (e.g., which may also be referred to as mechatronics system 700), roll rotation of the distal manipulator 560 is transmitted and controlled mechatronically. The term mechatronic here signifies a system that employs mechanical, electromechanical, and electronic components. The device 2010 receives inputs from the user to control roll with the roll input 526.

The user turns the roll dial 526 on the handle 520 with respect to the handle 520 about axis 222-lon. This rotation is detected (e.g., sensed, captured, measured, transduced) by the roll dial 526, which may be contained within the handle 520 (e.g., the first link 522a thereof). The roll dial encoder 526a produces (e.g., outputs) a roll dial encoder signal 526a′. The roll dial encoder signal 526a′ is typically one or more electrical signals, which can be digital and/or analog. In another embodiment, instead of a roll dial encoder 526a may be replaced with another type of sensor that detects rotation of the roll dial 526 and produces electrical signals (e.g., the roll dial encoder signal 526a or equivalent), such as a rotary potentiometer or a resolver.

This roll dial encoder signal 526a′ can be communicated to a roll dial encoder line driver 2126b that boosts the signal (and possibly inverts it) allowing it to be communicated over longer cable lengths while minimizing noise, electromagnetic interference, and signal data corruption. A roll dial encoder line receiver (not shown) can be installed at the receiving end of the boosted signal (e.g., functioning as an input to the microcontroller 2184a, in the case that differential signals are implemented, which will further minimize the undesirable effects (e.g., interference, corruption, or noise) in the transmitted roll input signal 2126a′. The use of a roll dial encoder line driver 2126b and receiver is optional but may be recommended, especially when long cables are involved. It is possible to only use the roll dial encoder line driver 2126b, or to use both the roll dial encoder line driver 2126b and receiver, or neither. Note that other various methods of mitigating undesired electromagnetic noise or interference are also possible, such as the use of electromagnetic shielding, passive filtering, twisted wires, and differential inputs etc.

As the roll input signal 2126a′ reaches the microcontroller 2184a, the microcontroller 2184a then determines according to the roll input signal 2126a′ the instantaneous angular position of the roll dial 2126a, which reflects the user input (i.e. how much roll rotation the user has commanded).

A separate encoder buffer board 2126d (or simply an encoder buffer) may be arranged between the roll dial encoder 2126a and microcontroller 2184a, or the roll dial encoder line receiver 2126c and the microcontroller 2184a (in case a roll dial encoder line receiver 2184a is used). The encoder buffer 2126d receives the roll input signal 2126a′ and processes it to produce a modified roll input signal 2126d′ at a suitably high rate to reduce the hardware or software computation burden in the microcontroller 2184a. Sometimes, the encoder buffer 2126d may be functionality built into the microcontroller 2184a, or may be a separate discrete electronic board or integrated circuit (IC). The encoder buffer 2126d then communicates the modified roll input signal 2126d′ (e.g., encoder counts) to the microcontroller 2184a via an interface (not labeled), which may be wired (e.g. Serial Peripheral Interface (SPI), Universal Asynchronous Receiver/Transmitter (UART), Inter-Integrated Circuit (I2C) etc.) or wireless (e.g. via Bluetooth, WiFi, radio, infrared etc.)

Upon receipt of the roll input signal 2126a′ or the modified roll input signal 2126d′, the microcontroller 2184a may perform various signal filtering, conditioning, and/or processing methods (e.g. low pass filters, high pass filters, moving average filters, saturation, band-pass/band-stop filters etc.) to reduce and/or smooth out remaining noise in the roll input signal 2126a′ or the modified roll input signal 2126d′ from sources such as electromagnetic interference and mechanical vibrations etc.

Separately, the roll motor 1760a housed in the housing 540c provides the electromechanical actuation to roll rotate the shaft and ultimately the end-effector) also has a roll motor encoder 2160b attached to it. The roll motor encoder 2160b detects (e.g., captures, measures, senses, transduces) the actual roll rotation of the roll motor output shaft (and therefore, the roll rotation of the shaft 542 and the gear train 1760b, and therefore the roll rotation of the distal manipulator 560.

The motor encoder 2160b produces (e.g., outputs) a motor output signal 2160b′. The motor output signal 2160b′ may be or include one or more electrical signals, which can be digital and/or analog. The motor encoder signal 2160b′ can be communicated to the motor encoder line driver 2160c that boosts the signal (and possibly inverts it) allowing it to be communicated over longer cable lengths while minimizing noise, electromagnetic interference, and signal data corruption. A motor encoder line receiver (not shown) can be installed at the receiving end of the boosted signal, in the case that differential signals are implemented, which will further minimize the undesirable effects (e.g., interference, corruption, or noise) in the transmitted motor output signal 2160a′. The motor encoder line driver 2160c and the receiver 2160d are also optional but may be advantageous, especially when long cables are involved. It is possible to only use the motor encoder line driver 2160c, or to use both the motor encoder line driver 2160c and receiver 2160d, or neither. Note that other various methods of mitigating undesired electromagnetic noise and/or interference are also possible, such as the use electromagnetic shielding, passive filtering, twisted wires, differential inputs, etc.

As the motor output signal 2160a′ reaches the microcontroller 2184a, the microcontroller 2184a then determines the instantaneous actual motor shaft roll rotation position, and using an appropriate scaling factor geartrain 1760b) within the microcontroller 2184a, the microcontroller 2184a determines what the actual roll rotation of the distal manipulator 560 is (i.e., the rotational position and/or number of rotations).

The motor encoder buffer board 2160d may advantageously be arranged between the motor encoder 2160b and the microcontroller 2184a, or the motor encoder line receiver and the microcontroller 2184a (in case an encoder line receiver is used). The motor encoder buffer board 2160d receives the motor encoder signal 2160a′ and processes it to produce a modified motor output signal 2160d′ and reduce the hardware or software computation burden in the microcontroller. The motor encoder buffer board 2160d functionality may be built into the microcontroller 2184a, and or may be a separate discrete electronic board or integrated circuit (IC). The motor encoder buffer board 2160d then communicates the modified motor output signal 2160d′ (e.g., with motor encoder counts) to the microcontroller 2184a via an interface that may be wired (e.g. Serial Peripheral Interface (SPI), Universal Asynchronous Receiver/Transmitter (UART), Inter-Integrated Circuit (I2C) etc.) or wireless (e.g. via Bluetooth, WiFi, Radio, Infrared etc.).

Upon receipt of the motor output signal 2160a′ or the modified motor output signal 2160d′, the microcontroller 2184a may perform signal filtering, conditioning and/or processing methods (e.g. low pass filters, high pass filters, moving average filters, saturation, band-pass/band-stop filters etc.) to reduce and/or smooth out remaining noise from sources such as electromagnetic interference, mechanical vibrations etc.

The microcontroller 2184a compares the actual roll rotation of the end-effector (coming from the motor encoder 2160b) with the user commanded value of the roll rotation (coming from the roll dial encoder 2126a) and based on this “difference” or “error” implements a control logic (a.k.a. control algorithm) that is designed and executed to minimize this “difference” or “error” to an acceptably small level. As such, even if the user holds the commanded value of roll rotation constant, but there is an external disturbance torque attempting to roll the end-effector away from the user's commanded value, the microcontroller 2184a will, via the control logic, counteract this external disturbance torque to maintain the roll rotation of the end-effector at the user's commanded value.

The control logic runs on the microcontroller 2184a, and can be based on feedback control, or feedforward control, or a combination of the two. This control logic can employ classical controls (e.g. PID controller, lead-lag controller), or modern controls (e.g. state space controller), or a combination thereof. This control logic can involve command shaping, input filtering, fuzzy logic, adaptive controls, and even machine learning (e.g. artificial intelligence) based controls. This control logic can include various Boolean logic (e.g. if, then, else statements) that captures different use case scenarios to accordingly switch the logic. The control logic includes various safety features to avoid accidental and/or unintentional driving of the roll motor 1760a, detecting faulty measurements from the motor encoder 2160b and/or the roll dial encoder 2126a, detecting unintended rotation of the roll motor 1760a and by extension the distal manipulator 560, limiting the voltage and/or current sent to the roll motor, limiting the amount of rotation and/or torque produced by the roll motor, among various other safety measures.

The microcontroller 2184a, according to the control logic, sends a suitable command signal 2184a′ to a motor driver 2184d. It may be desirable to implement various command shaping methods (e.g. low pass filters, high pass filters, moving average filters, saturation, band-pass/band-stop filters etc.) on the command signal 2184a′ before sending it to the motor driver to avoid unwanted behaviors such as abrupt motor fluctuations, resonance, overcurrent, and more. When the motor driver 2184d receives the command signal 2184a′, the motor driver 2184d sends a suitable amount of voltage, current, and/or power to the roll motor 1760a to turn the roll motor 1760a such that the above mentioned “difference” or “error” between the distal manipulator 560 rotation and roll dial rotation (as commanded by the user) is minimized. The motor driver 2184d receives command signals 2184a′ from the microcontroller 2184a and electrical power from a power source 2184e such as a battery, or other electrical power source 2184e (e.g. power adapter, AC to DC converter, transformer, etc.).

In general, one or more electrical power sources 2184e, and preferably a battery or multiple batteries are needed to power all these electronic components (e.g. microcontroller 2184a, encoder line driver, encoder buffer board, motor driver) and electromechanical components (roll dial encoder, motor encoder, roll motor) in the system 2000. Sometimes, there is also a need for stepping down/stepping up voltage for different electronic components. In this case, voltage regulators/converters can be used to provide power at a steady, precise voltage.

Any communication of signals discussed here can be wired (i.e. via electrical cables, or optical cable) or wireless (i.e. via Bluetooth, WiFi, radio, infrared etc.). However, all signal communication shown in FIG. 20A is via electrical cables.

Now let us proceed to describe one specific embodiment of the wristed apparatus (including the device 2010 and controller box 2080) that is shown in FIG. 20A.

In this embodiment, there is a power source, a battery, which is located inside the controller box.

In this embodiment, there is a roll dial encoder housed between the roll dial and handle body, and a roll dial encoder line driver located in the handle body. The roll dial encoder signal is routed from the roll dial encoder to the encoder line driver within the handle body, and then via internal electrical cables running through the handle body, pitch link, pitch joint, yaw link, yaw joint, frame arch, and ultimately to the frame body. This roll dial encoder signal is next routed from the frame box to the controller box via an external electrical cable shown in FIG. 20A. Within the controller box, this roll dial encoder signal goes to the encoder buffer board, which counts/decodes the signal and further routes it to the microcontroller, all within the controller box. The microcontroller then converts the decoded roll dial encoder signal into the rotation of the roll dial using a known conversion factor. The microcontroller then employs signal filtering/conditioning/processing methods to clean up and smooth out the rotation measurement of the roll dial before it is fed into the control logic. Note that the scaling factor between the roll dial encoder signal and roll dial can be varied to change the transmission ratio between roll dial rotation and end effector/shaft rotation.

The roll dial encoder and its associated roll dial encoder line driver, both within the handle sub-system, receive power from the battery located with the controller box. This electrical power is routed from the controller box to the frame box via an external electrical cable (shown in the figure above). Once within the frame box, this electrical power is routed via internal electrical cables running from the frame box, through the frame arch, yaw joint, yaw link, pitch joint, pitch link and ultimately to the handle body, where the roll dial encoder and its associated roll dial encoder line driver are located.

The “external electrical cable” 2090 mentioned here and shown in the above figure typically comprises a bundle of multiple individual electrical cables or wires. These multiple wires separately communicate individual signals, power (e.g. electrical voltage), electrical ground reference, etc.

Next, there is a roll motor with a motor encoder mechanically attached to it, all within the frame box. The motor encoder receives electrical power from the battery located in the controller box. This electrical power is routed from the controller box via the above mentioned external electrical cable(s) to the frame box. The motor encoder signal is routed from the motor encoder to an associated motor encoder line driver within the frame box. From there, this motor encoder signal is routed from the frame box to the controller box via the above mentioned external electrical cable(s). Within the controller box this motor encoder signal is received at the encoder buffer board, which further communicates the decoded motor encoder signal to the microcontroller. The microcontroller then converts the decoded motor encoder signal into shaft/end effector roll rotation using a known, constant conversion factor. Note that the scaling factor between the roll dial encoder signal and roll dial can be varied to change the transmission ratio between roll dial rotation and end effector/shaft rotation.

Next, the microcontroller feeds the end effector roll rotation and roll dial rotation into its control logic to determine a suitable command signal to minimize the “error” or “difference” between the two measured rotations. The command signal then undergoes conditioning/filtering/processing within the microcontroller to limit the maximum magnitude of commanded voltage/current/power. A motor driver, which is located within the controller box, receives power from the battery (located within the controller box), and receives a command signal from the microcontroller via internal electrical wiring/connections, all within the controller box. Based on this command signal from the microcontroller, electrical power is modulated and routed by this motor driver in the controller box to the roll motor in the frame box via an external electrical cable (seen in the above figure).

In general, the power from the motor driver (in the controller box) to the roll motor (in the frame box) may be routed via a second separate external electrical cable, i.e. separate from the external electrical cable that is used to route power and signals pertinent to the encoders between the device (specifically housing/frame box) and the controller box. It is generally a good practice to physically separate motor power cable from encoder signals and power cable to minimize/prevent electromagnetic interference and data corruption of the encoder signals.

While the above embodiment describes one power source for the entire wristed apparatus—a battery located in the controller box; in general, there can be multiple power sources located in the various modules of the wristed apparatus. For example, there could be batteries preferably located within one or more of the hand body, pitch link, yaw link, frame arch, and frame box, etc. When there is one or more power source within the apparatus, there can be various voltage regulators within the system that regulate the source voltage down any lower voltage level (e.g. 3.3V or 5V) that might be needed by the any of the electronic components needed above. Sometimes, power is provided directly to an electronic or electromechanical component, or indirectly via a voltage regulator. At other times, one or more power source(s) within the wristed apparatus may power a microcontroller, which in turn may have an internal voltage regulator, and may be used to power some or all of the electronic and electromechanical components in the system.

While the above embodiment describes the electrical cable to transmit data signals and power supply lines separately in individual cables or wires, it is also possible to transmit both data signals and power supply lines together in the same cables or wires using technologies such as Power Over Ethernet (PoE).

While the above embodiment describes only the primary function of the controller box, i.e. to house the microcontroller, battery, encoder buffer board, motor driver, the controller box can also contain a range of displays and user interfaces. For example, while maintaining the same or substantially similar functionality described above, the controller box may contain additional functions such as user interfaces to display battery life, switches to allow selection of different use modes, components to track device usages, etc.

While the above embodiment describes one arrangement or configuration of the electronic and electromechanical components, housed within the device and/or controller box, there are many alternative embodiments or configurations that are possible. For example, while maintaining the same or substantially similar functionality described above, all the electronic and electromechanical components mentioned above machine may be suitably packaged and housed within one or more of the handle sub-assembly, pitch link, yaw link, frame arch, or the frame box. In such configurations, there will not be an external controller box and the entire wristed apparatus will visually look like the device in the above figure. However, it is to be understood that all the physical components (electronic, electromechanical, and mechanical) can be housed, packaged, mechanically assembled, electrically connected all within the device. In the present context, “robotic” and “mechatronic” are generally interchangeable terms.

In the present context, “electrical” and “electronic” are generally interchangeable terms.

Referring to FIGS. 5B and 6, the handle 520 may further include a tool input 528, which is configured to receive user inputs for controlling the tool outputs provided by the distal manipulator 560. The tool input 528 may, as shown, be a retractable lever (or trigger) that is depressible and releasable by the user to operate the distal manipulator 560, for example, to close and open the distal manipulator 560, respectively. Alternatively, the tool input 528 may be a push button, a latch, a ratcheted input, a scissor-grip, a thumb button, a squeeze ball, etc. As shown in FIG. 7, the movement of the tool input 528 is transmitted mechanically by the input transmission 524 from the tool input 528 to the intermediate transmission 544. For example, the input transmission 524 includes an input tool cable 728a that extends from the handle 520 to the intermediate transmission 544 through the first link 522a, the first pivot joint 522c, the second link 522b, and the second pivot joint 522d into the chassis 540. The intermediate transmission 544 receives, converts, and transmits movement of the input tool cable 728a to the output transmission 564 that in turn causes the distal manipulator 560 to provide the specific output, such as by opening or closing the jaws thereof. A key feature of this cable transmission is that as the tool input cable 728a has to traverse through the first pivot joint 522c and the second pivot joint 522d to reach the chassis 540, the tool input cable 728a should not be influenced (or be minimally influenced) by the rotations of the first pivot joint 522c and/or the second pivot joint 522d. To accomplish this decoupling, several strategies can be employed. One strategy is to route the cables 728a as close as possible to the rotation axis 522c′ of the first pivot joint 522c and the rotation axis 522d′ of the first pivot joint 522c. Another strategy is to use a Bowden cable arrangement wherein the cable 728a is routed via a flexible sheath, as it extends from the handle 520 into the chassis 540.

As shown in FIG. 8, movement of the tool input 528 may instead be transferred to the distal manipulator 560 mechatronically. In this case, the input transmission 524 includes a sensor 828a, such hall sensor or potentiometer or pressure sensor or touch sensor, that measures the position of or force applied on the tool input 528 relative to the handle 520 and sends electronic signals 828a′, which may be referred to as tool input signals, according to the position to a microcontroller. The microcontroller in turn commands an electromechanical actuator (e.g. motor) to produce a tool input that the intermediate transmission 544 then receives and converts the tool input signal 828a′ by transmitting movement to the output transmission 564 that causes the distal manipulator 560 to provide the tool output, such as by opening or closing the distal manipulator 560.

In the case of implementing sensors for sensing articulation inputs, the articulated roll input, or the tool input, the sensors may be electrically connected to the intermediate transmission 544 via a wire to be powered thereby and transmit signals therebetween. Alternatively, the sensors may be powered by a battery and transmit signals wirelessly with the intermediate transmission 544.

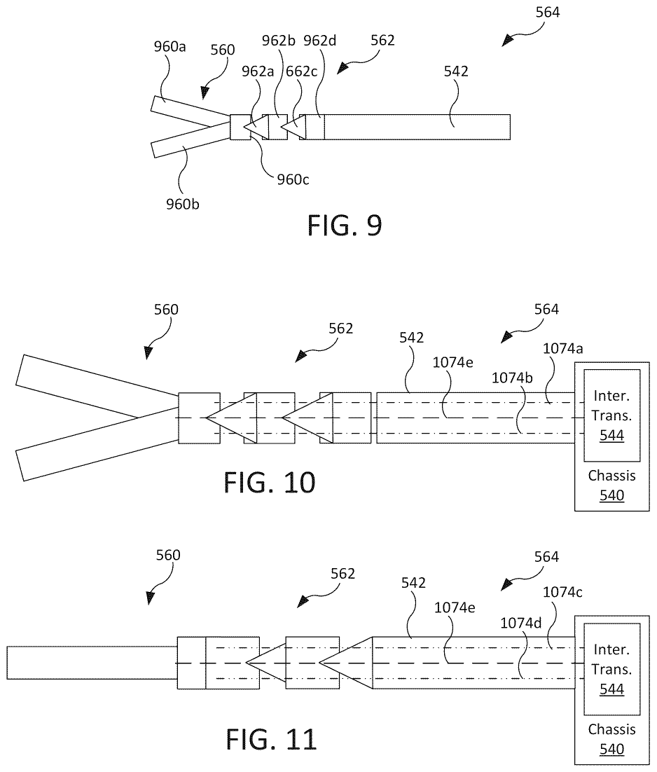

Referring to FIGS. 9-15, the tool (i.e. the distal manipulator) 560 may, for example, be configured as a pair of jaws 960a, 960b that are actuated to move toward each other so as to close and away from each other so as to open. A proximal end 960c of the distal manipulator 560, which is coupled to the shaft 542 by way of the output articulation joint 562. The shaft 542, the output articulation joint 562, and the distal manipulator 560 are rotatably constrained to each other relative to the device longitudinal axis 112-lon, such that rotation of the shaft 542 thereabout relative to the chassis 540 causes rotation of the output articulation joint 562 and the distal manipulator 560 relative to the chassis 540. In this manner, each of the shaft 542 and the output articulation joint 562 may transfer roll output from the intermediate transmission 544 to the distal manipulator 560 and, accordingly, be considered part of the output transmission 564, as discussed in further detail below.

The output articulation joint 562 facilitates articulation of the distal manipulator 560 relative to the shaft 542. As shown in FIG. 9, the output articulation joint 562 may include a series of pivot members 962a-d, such as two or three or four pivot members, that are connected in series and pivoted relative to each other about axes that alternate in direction between being parallel with the output lateral axis 262-lat and the output transverse axis 262-tra.

As shown in FIGS. 10-11, the output transmission 564 transfers output motion from the intermediate transmission 544 to the distal manipulator 560 to articulate, roll, and provide the tool output according thereto. The output transmission 564 may, for example, include two sets of mechanical cable lengths. A first set includes first and second output cable lengths 1074a, 1074b that are pulled by the intermediate transmission 544 to bend the output articulation joint 562 and, thereby, articulate the distal manipulator 560 relative to the output lateral axis 262-lat to pitch the distal manipulator 560 relative to the shaft 542 and chassis 540. A second set includes first and second output cable lengths 1074c, 1074d that are pulled by the intermediate transmission 544 to bend the output articulation joint 562 and, thereby, articulate the distal manipulator 560 relative to the output transverse axis 262-tra to yaw the distal manipulator 560 relative to shaft 542 and the chassis 540. As illustrated in both FIGS. 10-11, the output transmission 564 may also include an output tool cable 1074e that extends centrally through the shaft 542 and the output articulation joint 562 to the distal manipulator 560. The intermediate transmission 544 pulls on the output tool cable 1074e to cause the jaws 960a, 960b to move toward each other, so as to close the distal manipulator 560. The jaws 960a, 960b may be sprung open, such that release of tension in the tool cable 1074e allows the jaws 960a, 960b to spring apart. This spring action can be accomplished in the distal manipulator or may be implemented via a second output tool cable that extends back from the distal manipulator, centrally through the output articulation joint 562 and the shaft 542, into the intermediate transmission 544, terminating at an distal manipulator jaw opening spring within the intermediate transmission.

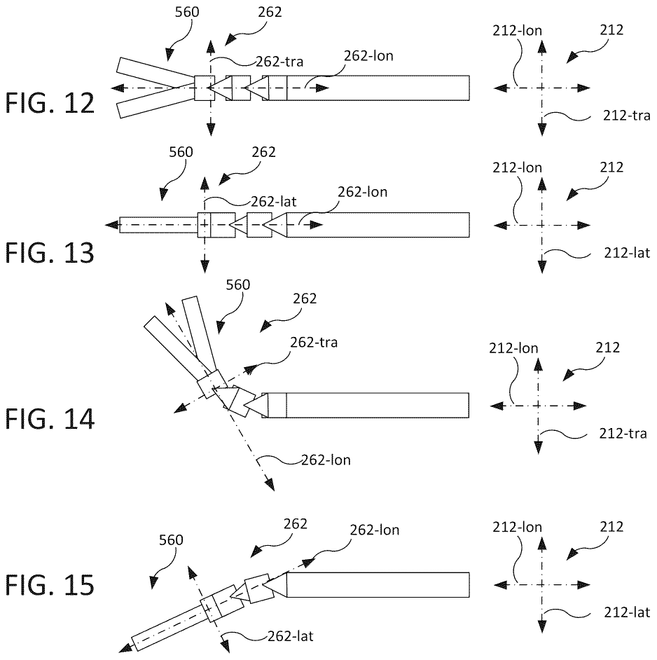

As illustrated in FIGS. 12-15, as referenced above, the output frame of reference 262 is associated with the distal manipulator 560, for example, with the origin of the output frame of reference 262 coinciding with the proximal end 960c of the distal manipulator 560. As also referenced above, the device frame of reference 212 is associated with the shaft 542, for example, with the device longitudinal axis 212-lon being coaxial with the shaft 542. The distal manipulator 560 is movable between the nominal orientation (e.g., in which the output longitudinal axis 262-lon thereof is parallel with the device longitudinal axis 212-lon) and one or more (e.g., variable) articulated orientations (e.g., in which the output longitudinal axis 262-lon is not parallel with the device longitudinal axis 212-lon). The distal manipulator 560 may rotate about the device longitudinal axis 212-lon that the shaft 542 rotates about. FIGS. 12 and 13 are side and top views of the distal manipulator 560 in the reference orientation. FIGS. 14 and 15 are side and top views of the distal manipulator 560 in an example articulated orientation articulated upward (e.g., positively in pitch) and leftward (e.g., positively in yaw).

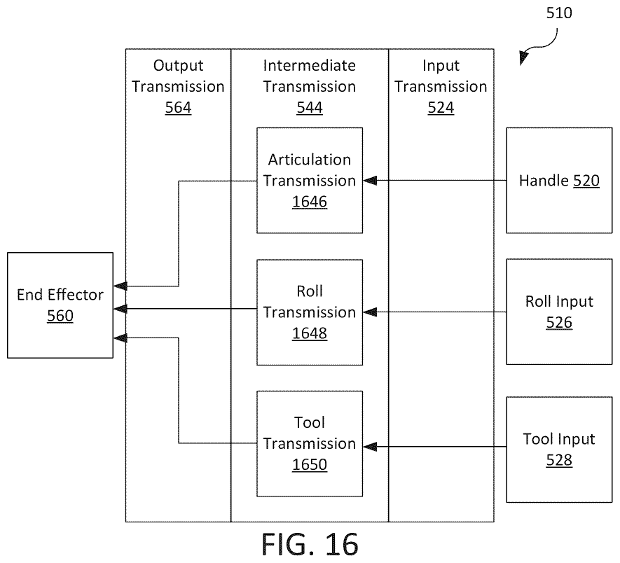

Referring again to FIGS. 5A and 16, as referenced above, the intermediate transmission 544 receives user inputs from the handle 520 via the input transmission 524, converts the user inputs into physical outputs, and transfers the physical outputs to the distal manipulator 560 via the output transmission. Referring to FIG. 16 intermediate transmission 544 may be considered to generally include multiple transmissions that convert the inputs into the outputs. For example, the intermediate transmission 544 may include an articulation transmission 1646, a roll transmission 1648, and a tool output transmission 1650. The intermediate transmission 544 and the subsystems thereof may be partially or wholly contained within the chassis 540. The articulation transmission 1646 receives and converts articulation inputs from the handle 520 via the input transmission 524 into the articulation outputs transferred by the output transmission 564 to the distal manipulator 560 to provide the articulation thereof. The roll transmission 1648 receives and converts the articulated roll inputs from the articulated roll input 526 via the input transmission (accomplished mechatronically) into the articulated roll outputs transferred by the output transmission 564 of the distal manipulator 560 to provide roll thereof. As discussed in further detail below, the articulation transmission 1646 is configured to work in cooperation with the roll transmission 1648 to provide articulated roll of the distal manipulator 560. The tool output transmission 1650 receives and transfers tool operation inputs from the tool input 528 via the input transmission (accomplished mechanically or mechatronically) into outputs provided by the distal manipulator 560.

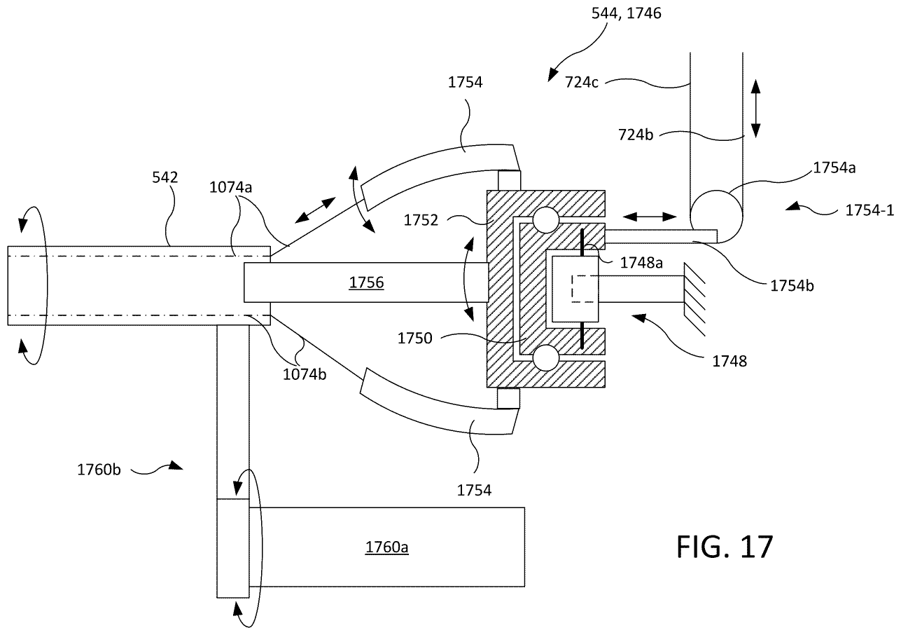

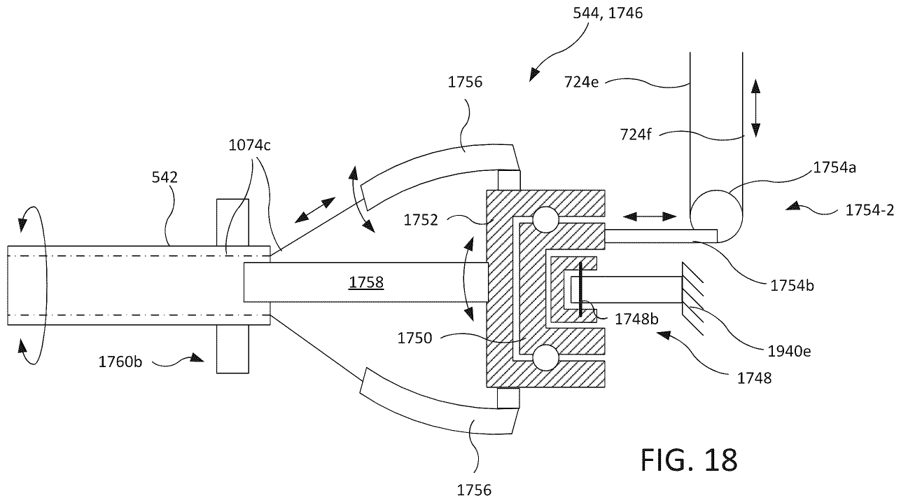

Referring to FIGS. 17-19, the articulation transmission 1646 is configured to receive the articulation inputs mechanically from the input transmission 524, mechanically convert the articulation inputs into articulation outputs, and mechanically transfer the articulation outputs to the output transmission 564. While the articulation inputs may be transferred mechatronically by the input transmission 524, any such articulation input is converted into a mechanical input received at the articulation transmission 1646.

In the embodiment shown, the articulation transmission 1646 is configured as a swashplate mechanism 1746. The articulation transmission 1646 (e.g., swashplate mechanism 1746) is generally configured to receive, convert, and transfer the articulation inputs from the handle 520 via the input transmission 524 to the distal manipulator 560 via the output transmission 564 to cause articulation thereof, while also maintaining the distal manipulator 560 in an articulated orientation as the distal manipulator 560 is rotated, so as to provide roll of the distal manipulator 560 about the articulated 262-lon axis (e.g., as the distal manipulator 560 is rotated by the roll transmission 1648, such as by the electric motor 1760a thereof). The distal manipulator 560 is rotatable with an unlimited range of motion, which may correspond to the unlimited range of motion of the output member 1752 (described below).

The swashplate mechanism 1746 generally includes a pivot 1748, an input member 1750, and an output member 1752. Generally speaking, the input member 1750 is configured to receive the articulation inputs from the input transmission 524 and transfers the articulation movement to the output member 1752, while the output member 1752 transfers the articulation movement from the input member 1750 to the output transmission 564 and, thereby, the distal manipulator 560 while also permitting the distal manipulator 560 to rotate while being maintained in an articulated orientation.

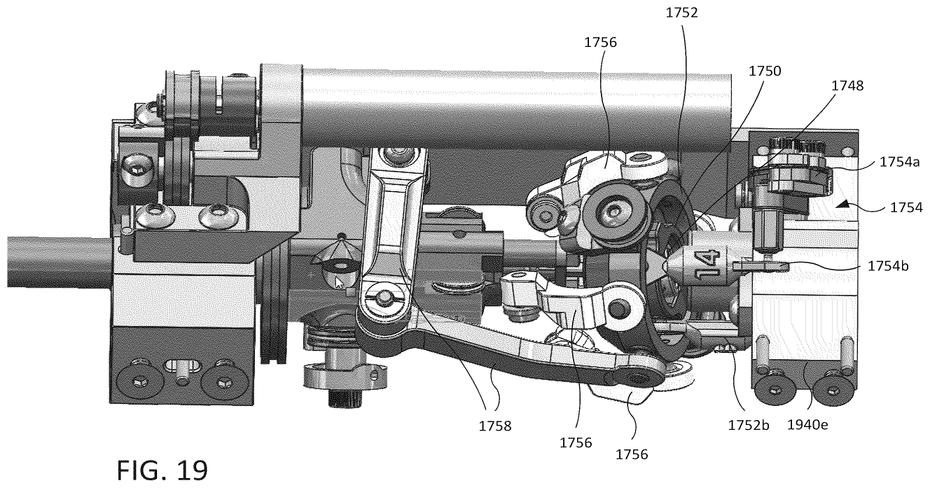

The pivot 1748 is coupled to a non-moving portion of the chassis 540 to form a ground reference associated with the device frame of reference 212. For example, as shown in FIG. 19, the chassis 540 may generally include a frame 1940e within the housing 540c (not illustrated). The housing 540c contains the swashplate mechanism 1746 therein, while the pivot 1748 is immovably coupled to the frame 1940e and, thereby, fixed relative to the device frame of reference 212. The input member 1750 is pivotably supported by the pivot 1748 relative to the device frame of reference 212 (e.g., within the housing 540c) and, thereby, pivotable about a first pivot axis 1748a, a second pivot axis 1748b, or both that are perpendicular to each other. In some embodiments, axes 1748a and 1748b may correspond to device frame axes 212-lat and 212-tra. The frame 1940e of the chassis 540 may include several members that are immovably coupled to each other and to which movable components of the input transmission 524, the intermediate transmission 544, and the output transmission 564 are coupled. The housing 540c is spaced apart from the output member 1752 to move between the other nominal and articulated orientations, to rotate in the articulated orientations (e.g., is rotatable an unlimited rotational range of motion relative to the housing 540c), or both freely of (i.e., without engaging) the housing 540c. The device frame of reference 212 may be fixed relative to the housing 540c, while the output member 1752 is pivotable relative to the housing 540c about a device pitch axis thereof (i.e., one of the device lateral or transverse axes 212-lat, 212-tra between the other nominal orientation and the other articulated orientations.

The input member 1750 is additionally constrained by the pivot 1748 from any other translational or rotational movement relative to the device frame of reference 212. Specifically, the pivot 1748 and the input member 1750 are cooperatively configured for the input member 1750 to pivot relative to the device frame of reference 212 in correspondence with the pitch, yaw, or both of the distal manipulator 560 but to not pivot or otherwise rotate relative to the device frame of reference 212 in correspondence with roll of the distal manipulator 560. For example, the input member 1750 may be constrained from rotating about the device longitudinal axis 212-lon or an axis parallel therewith. The pivot 1748 may, for example, be a two-axis gimbal. Alternatively, in some embodiments, the pivot 1748 may be a ball and socket joint. As illustrated, the input member 1750 may be a cup-shaped member in which the pivot 1748 is positioned. The input member 1750 may also be referred to as a non-rotating or first member, plate, component, or structure.

The input member 1750 receives the articulation inputs from handle 520 via the input transmission 524, for example, via the cable lengths 724b, 724c, 724e, 724f to pivot (i.e. articulate or rotate) the input member 1750 about the pivot 1748. When in a reference orientation relative to the chassis 540 (e.g., to the device frame of reference 212), which corresponds to the reference orientation of the distal manipulator 560, a longitudinal axis (not illustrated) of the input member 1750 and/or the output member 1752 may be parallel or coaxial with the device longitudinal axis 212-lon. When in articulated orientations, which correspond to articulated orientations of the distal manipulator 560, the longitudinal axis of the input member 1750 may not be parallel with the device longitudinal axis 212-lon. Stated differently, the input member 1750, the output member 1752, or both are movable between other reference and articulated orientations that correspond to the reference and articulated orientations of the distal manipulator 560. In the nominal orientations, the distal manipulator 560, the output member 1752, or both may rotate about the device longitudinal axis 212-lon that the shaft 542 also rotates about.

The input cable lengths 724e, 724f, 724c, 724b of the input transmission 524 are directly or indirectly coupled to the input member 1750, such that as the input cable lengths 724c, 724b, 724e, 724f translate, the input member 1750 pivots about the first and second pivot axes 1748a, 1748b of the pivot 1748. In one example, the input cable lengths 724c, 724b, and the input cable lengths 724e, 724f are indirectly coupled to the input member 1750 via a first transfer mechanism 1754-1 and a second transfer mechanism 1754-2, respectively. Each of the first and second transfer mechanisms 1754-1, 1754-2 may be considered part of the input transmission 524 and generally includes a pulley 1754a and a drive link 1754b. The pulley 1754a is coupled the input cable lengths 724c, 724b or the input cable lengths 724e, 724f, to be rotated thereby. The drive link 1754b is a rigid member that extends between the pulley 1754a and the input member 1750 to transfer motion of the cable lengths to the input member 1750. Each of the drive links 1754b is coupled at one end with a ball joint to the pulley 1754a at a position on the pulley 1754a that is radially outward of a rotational axis thereof. Another end of the drive link 1754b is coupled with another ball joint to the input member 1750 of the swashplate mechanism 1746 at a circumferential position disposed away from one of the pivot axes 1748a, 1748b of the pivot 1748 corresponding thereto. The drive links 1754b of the two transfer mechanisms 1754 may be coupled to the input member 1750 at positions spaced apart by approximately 90 degrees about the longitudinal axis of the input member 1750. As each of the input cable lengths 724c, 724b or the input cable lengths 724e, 724f is pulled from articulation inputs to the handle 520, the pulley 1754a is rotated and the drive link 1754b pushes or pulls the input member 1750 of the swashplate mechanism 1746 causing it to pivot (or articulate) about the first pivot axis 1748a, the second pivot axis 1748b, or both. Each of the drive links 1754b of the input transmission 524 may be referred to as input links.

In the case of the articulation inputs being electronic inputs, the transfer mechanisms 1754-1, 1754-2 may instead include motors (not shown) that are configured to rotate the pulleys 1754a and, thereby, move the drive links 1754b according to the articulation input signals. In such case, the transfer mechanisms 1754 may still be considered part of the input transmission 1724 and mechanically transfer the articulation inputs to the swashplate mechanism 1746 and, thereby, the intermediate transmission 544.

The output member 1752 is rotatably coupled to the input member 1750 about a roll rotation axis. More particularly, the output member 1752 is rotatable about the longitudinal axis of the input member 1750 and may have an unlimited rotational range of motion (i.e. rotation) thereabout. As referenced above, when the swashplate mechanism 1746 is in a reference orientation, the longitudinal axis of the input member 1750 may be coaxial or otherwise parallel with the device longitudinal axis 212-lon. Other than rotation about the longitudinal axis of the input member 1750, the output member 1752 is constrained in all other movement relative to the input member 1750. As a result, as the input member 1750 is pivoted according to the articulation inputs about the pivot 1748, the output member 1752 is also pivoted by generally the same amount about the pivot 1748. For example, as illustrated, the output member 1752 may be a cup-shaped member in which is received the input member 1750. The output member 1752 may be rotatably coupled to the input member 1750 via bearings, such as ball bearings in circumferential tracks of the input member 1750 and the output member 1752, which further constrain all other movement of the output member 1752 relative to the input member 1750.

The output member 1752 transfers the articulation of the input member 1750 to the distal manipulator 560 via the output transmission 564, for example, via first and second output cable lengths 1074a, 1074b and the first and second output cable lengths 1074c, 1074d. The pairs of output cable lengths 1074a, 1074b and 1074c, 1074d are directly or indirectly coupled to the output member 1752, such that as the output member 1752 pivots with the input member 1750 about the pivot axes 1748a, 1748b, the output cable lengths 1074a, 1074b, 1074c, 1074d translate along their respective lengths and cause the distal manipulator 560 to articulate.

In one example, the output cable lengths 1074a, 1074b, 1074c, 1074d are each indirectly coupled to the output member 1752 via coupling links 1756. Each of the coupling links 1756 is a generally rigid member that is coupled to the output member 1752 with a ball joint, for example, to an outer circumferential surface thereof as shown. Each of the coupling links 1756 may further be curved or otherwise include a radially-outward curved shape (e.g., defining an inward recess) that, as the output member 1752 is pivoted about the first and second pivot axes 1748a, 1748b, permits movement of the output member 1752 therein without interference therebetween. As the output member 1752 is pivoted by the input member 1750 the sets of output cable lengths 1074a, 1074b and 1074c, 1074d are translated and, thereby, cause articulation of the distal manipulator 560. That is, the output cable lengths 10741, 1074b, 1074c, 1074d are coupled to the output member 1752 and the distal manipulator 560, such that as the output member 1752 is moved between the its nominal and articulated orientations, the distal manipulator 560 is moved by the output cable lengths 1074a, 1074b, 1074c, 1074d between its nominal and articulated orientations, respectively.

As referenced above, the output member 1752 is rotatable relative to the input member 1750. More particularly, the output member 1752 is rotatably coupled to the shaft 542, so as to transfer torque therebetween rotate in unison therewith relative to the chassis 540, for example, about the device longitudinal axis 212-lon. Furthermore, the output cable lengths 1074a, 1074b, 1074c, 1074d extend from the output member 1752 through the shaft 542 and rotate therewith about the device longitudinal axis 212-lon. This ensures that the cable lengths 1074a, 1074b, 1074c, 1074d do not get coiled up or wound up within the shaft as the shaft rotates about the device longitudinal axis 212-lon. The shaft 542 receives torque from the roll transmission 1646 (e.g., the motor 1760a thereof) and transfers the torque to the output member 1752 to cause rotation thereof and, as referenced above, transfers torque to the distal manipulator 560 to cause rotation thereof. For example, the shaft 542 may transfer torque from the motor 1760a to the distal manipulator 560 and the output member 1752 in parallel to cause rotation thereof.

The shaft 542 is rotatably coupled to the output member 1752 of the swashplate 1744 with a roll coupling linkage 1758, which may also be referred to as a roll drive linkage. The roll coupling linkage 1758 is coupled to and transfers motion (e.g., torque) between the shaft 542 and the output member 1752. The roll coupling linkage 1758 is also configured to accommodate pivoting of the output member 1752 about the pivot 1748. For example, as shown, the roll coupling linkage 1758 may include a first link coupled to the shaft 542 and a second link extending from the first link and coupled to the output member 1752. A first end of the linkage 1758 is coupled of the shaft 542 (e.g., formed by a first link), while a second end of the linkage 1758 is coupled to the output member 1752 (e.g., formed by a second link). The linkage 1758 is configured to accommodate changing distances between the first end and the second end as the output member 1752 rotates while in articulated orientations. The first end of the linkage rotates at a fixed radial distance about the device longitudinal axis 212-lon, and the second end rotates at variable radial distances about the longitudinal axis as the output member 1752 rotates in the articulated orientations. The first end of the linkage 1758 is coupled to the shaft at a fixed longitudinal position along the device longitudinal axis 212-lon, and the second end of the linkage varies in longitudinal distance positions along the longitudinal axis as the output member rotates in the other articulated orientation. The first link is coupled to the shaft 542 with a pivot joint having a first pivot axis that is generally perpendicular to and spaced radially outward from a rotational axis of the shaft 542 (e.g., the device longitudinal axis 212-lon). The first link extends generally perpendicular to the first pivot axis and the rotational axis of the shaft 542, although the latter is not necessary. The second link is coupled to the first link with another pivot joint having a second pivot axis that is generally parallel with the first pivot axis and may be positioned on a radially opposite side of the shaft 542 from the first pivot axis. The second link extends rearward toward the output member 1752 and is coupled to the output member 1752 with a ball joint.

As the shaft 542 and the output member 1752 of the swashplate mechanism 1746 are rotated by the roll transmission 1648, the output member 1752 is held in an articulated orientation by the input member 1750 according to the articulation inputs, thereby providing articulated roll of the distal manipulator 560. That is, the distal manipulator 560 is rotated while in the one or more articulated orientations. As the output member 1752 rotates, the output cable lengths 1074a, 1074b, 1074c, 1074d orbit around the rotational axis (e.g., the device longitudinal axis 212-lon) therewith changing their rotational position about the input member 1750 and, thereby, change their longitudinal position relative to the device longitudinal axis 212-lon. As a result, as the shaft 542 rotates about the device longitudinal axis 212-lon, the output cable lengths 1074a, 1074b, 1074c, 1074d translate relative to the shaft 542, thereby causing the distal manipulator 560 to articulate relative to the shaft 542 as it rotates and, thereby, maintain the articulated orientation of the distal manipulator 560 relative to the tool reference frame 212.

As referenced above, the intermediate transmission 544 includes the roll transmission 1648. The roll transmission 1648 receives the roll inputs from the input transmission 524, for example, mechatronically and converts the roll inputs into roll outputs that are transferred by the output transmission 564 to the distal manipulator 560 to cause roll thereof. The roll transmission 1648 may include a motor 1760a (i.e. roll motor) that is operatively coupled to the shaft 542 to cause rotation thereof relative to the chassis 540. For example, the motor 1760a may apply torque to the shaft 542, which in turn transfers torque from the motor 1760a to the distal manipulator 560 to cause rotation thereof (e.g., in the nominal or articulated orientations). The shaft 542 is coupled to the frame 1940e of the chassis 540 with one or more bearings 1942a that permit the shaft 542 to rotate relative to the frame 1940e while preventing other movement therebetween (e.g., translational and/or pivoting movements). The motor 1760a is operatively coupled to the shaft 542 via a gear train 1760b or other rotation transfer mechanism (e.g. belt and pulleys, or chain and sprockets, etc.) that causes the shaft 542 to rotate relative to the frame 1940e of the chassis 540. The motor 1760a rotates the shaft 542 according to the roll input signals 726a′ received from the roll input sensor 526a that measures rotation of the roll input 526. For example, the roll input transmission 1648 may include a transducer configured to receive the articulated roll input signals 726a′, and a microcontroller that operates the motor 1760a via a motor driver to rotate the shaft 542 according to the roll input signals 726a′. For example, the motor 1760a may rotate the shaft 542 (and therefore the distal manipulator) by the same roll angular displacement that the roll input 526 is rotated relative to the handle 520.

As referenced above, the intermediate transmission 544 includes the tool output transmission 1650. The tool output transmission 1650 may be configured in any suitable manner, for example, including a length of cable or member extending between the input tool cable 728a and the output tool cable 1074e that mechanically transfers the tool input to the tool output. In the case of the tool input being transferred mechatronically from the tool input 528, the tool output transmission 1650 may include an actuator, such as a motor or voice coil, that pulls the output tool cable 1074e.

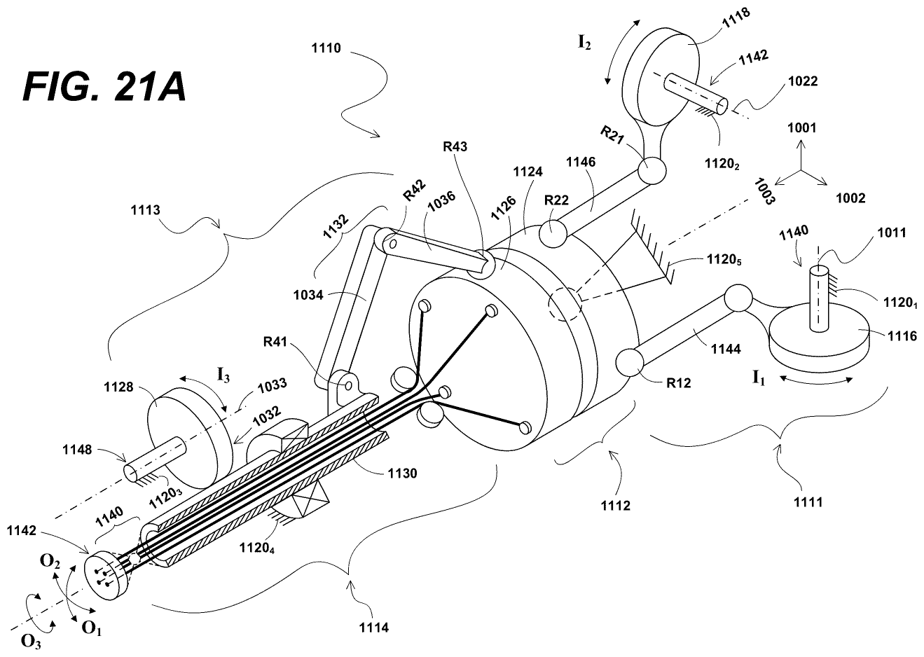

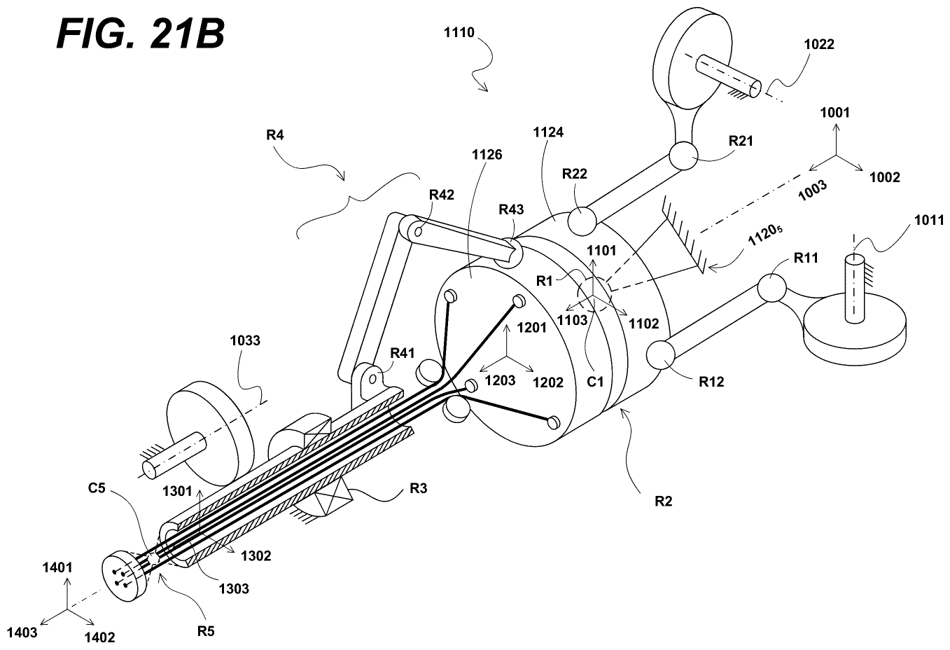

Referring now to FIGS. 21A and 21B, an exemplary intermediate module 140 is illustrated. The intermediate module is configured as a transmission system (i.e. a swashplate mechanism) 1110 capable of receiving three rotational inputs, each received at a respective input member (i.e. at the input end) and converting the rotational inputs into three rotational outputs delivered at a single output member (i.e. at the output end).

The transmission system 1110 comprises an articulation input sub-system 1111 (comprising at least two input members 1116, 1118), a swashplate sub-system 1112 (comprising at least two swashplates 1124, 1126), a roll input sub-system 1113 (comprising at least a third input member 1128, a shaft 1130, and a roll drive coupling 1132), and an output sub-system 1114 (comprising at least an output joint 1140 and an output member 1142). The proximal end of the transmission system 1110 is defined as being closer to the input sub-system 1111 while the distal end of the transmission system 1110 is defined as being close to the output sub-system 1142.

The three inputs are received separately at the three input members. For example, a first input I₁ is received at a first input member 1116, a second input I₂ is received at a second input member 1118, and a third input I₃ is received at a third input member 1128. The first input I₁ and the second input I₂ may be referred to as articulation inputs. In some examples, the first input I₁ and the second input I₂ may be orthogonal and may correspond to a yaw rotation and a pitch rotation. In other examples, the first input I₁ and the second input I₂ may not be orthogonal and may refer to other axes that produce tip and tilt. The third input I₃ may correspond to a roll rotation.

The corresponding outputs are provided at an output member 1142 (e.g. a distal manipulator), where a first output O₁ and a second output O₂ may be a yaw rotation and a pitch rotation, respectively, of the output member 1142. A third output O₃ may be a roll rotation of the output member 1142. Rotations of the distal manipulator in the first output O₁ and the second output O₂ together may be referred to as the “articulation of the distal manipulator”. Rotation in the third output O₃ may be referred to as the “roll rotation of the distal manipulator” or “distal manipulator roll”.

The transmission system 1110 may include a plurality of mechanical grounds. The grounds 1120 may be physical members such as a structure or chassis or base or frame, upon which the various other members of the system 1110 are mounted or coupled. For example, the first input member 1116 may be mounted to a first ground 1120₁, the second input member 1118 may be mounted at a second ground 1120₂, the third input member 1128 may be mounted at a third ground 1120₃, the shaft may be mounted at a fourth ground 1120₄, and the first and second swashplates 1124, 1126 may be mounted at a fifth ground 1120₅. The grounds are not fixed or immobile in an absolute sense. The grounds serve as a reference with respect to which the motions of the various members in the transmission system 1110 are described. The grounds may be a continuum or may be a structural arrangement of discrete components that are coupled to each other. The grounds may take any physical shape as required by the geometry, assembly, or application of the overall transmission system 1110. Axes 1001, 1002, 1003 are fixed to the grounds. Axes 1001, 1002, and 1003 may be perpendicular to each other and may intersect with each other.

Referring to FIGS. 21A and 21B, the transmission system 1110 comprises the swashplate sub-system 1112 which further comprises the first swashplate 1124 and the second swashplate 1126. The first swashplate 1124 is coupled to the ground 1120₅ of the transmission system via a first rotational joint R1 with a center of rotation at C1. The first rotational joint R1 may be a two-DoF (“degrees of freedom”) rotational joint (e.g. a universal joint, a cardan joint, a constant velocity or CV joint, etc.) or a three-DoF rotational joint (e.g. spherical joint, ball and socket joint, etc.). The first rotational joint R1 allows the first swashplate 1124 to have at least two articulation rotations (e.g. yaw and pitch) with respect to the ground 1120₅, and constrains the three translations of the first swashplate 1124 with respect to the ground 1120₅ at the center of rotation C1 of the rotation joint R1.

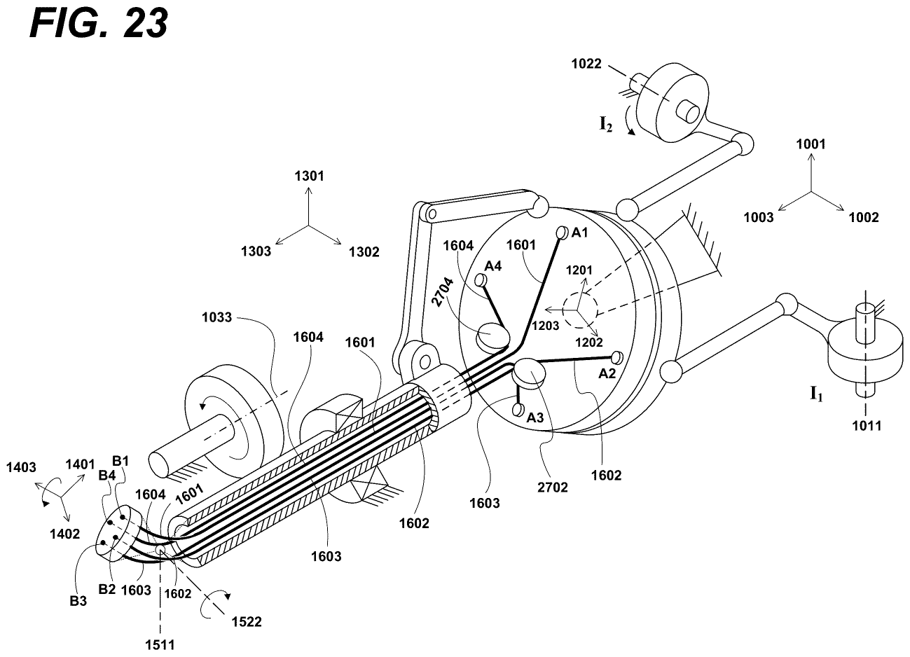

Axes 1101, 1102 and 1103 are assigned to the first swashplate 1124. Axes 1101, 1102, and 1103 may all intersect at the center of rotation C1 of the rotational joint R1 of the first swashplate 1124 such that the first swashplate 1124 can articulate in yaw and pitch rotations about axes 1101 and 1102, respectively. When the first swashplate 1124 is in its nominal (i.e. non-articulated) configuration (FIG. 21B), axis 1103 coincides with axis 1003. Furthermore, in this configuration, the plane formed by axes 1101 and 1102 is parallel to the plane formed by axes 1002 and 1003. In this configuration, axes 1001 and 1101 may be parallel, and axes 1002 and 1102 may be parallel. In an articulated configuration of the first swashplate 1124 (see for example, FIG. 23), axis 1103 is no longer collinear or parallel with axis 1003, and instead points at an angle with respect to axis 1003. Similarly, the plane formed by axes 1101 and 1102 is no longer parallel to the plane formed by axes 1001 and 1002.

The first swashplate 1124 is coupled to the second swashplate 1126 via a second rotational joint R2 that allows one rotational DoF (i.e roll rotation) about axis 1103 and constraints articulation (i.e. yaw and pitch rotations) about axes 1101 and 1102. Furthermore, in certain embodiments translations along the three axes may also be constrained by the second rotational joint R2, while in other embodiments that may not be the case. In some examples, the second rotational joint R2 may include one or more rolling element bearings (e.g. ball bearing, roller bearing), or one or more bushings to support axial, radial, and/or moment loads. The second swashplate 1126 articulates (in yaw and/or pitch rotations) along with the first swashplate 1124 by generally the same amount. In other words, the yaw and pitch rotations of the second swashplate 1126 are effectively the same as the yaw and pitch rotations of the first swashplate 1124. However, the rotational DoF allowed by the second rotational joint R2, allows the second swashplate 1126 the freedom to rotate about axis 1103 (i.e. roll rotation) with respect to the first swashplate 1124. More simply, the first swashplate 1124 and the second swashplate 1126 are capable of articulating in the yaw and/or pitch rotations, articulating at the same amount. However, the first swashplate 1124 does not rotate about axis 1103, while the second swashplate 1126 is capable of rotating about axis 1103 with roll rotation.

Axes 1201, 1202, and 1203 are fixed to the second swashplate 1126. In general, axes 1103 and 1203 remain collinear as the two swashplates 1124, 1126 articulate together and as the second swashplate 1126 rotates in roll rotation with respect to the first swashplate 1124. The plane formed by axes 1201 and 1202 remains parallel to the plane formed by axes 1101 and 1102, even as the plane formed by axes 1201 and 1202 rotates with respect to the latter plane about axes 1103 or 1203. In certain embodiments, axes 1201, 1202, and 1203 can all intersect at the center of rotation C1.

While the first 1124 and second swashplates 1126 are shown as disks or plates, they can take any other mutually similar or different shapes that might be necessitated by packaging and assembly constraints associated with other components and members in their vicinity. The shapes of the first and second swashplates can also be determined by the application at hand. These shapes may be rings (circular or square or rectangular), domes, hemispheres, or any other shapes. In certain embodiments and as illustrated in FIGS. 21A and 21B, the first swashplate 1124 is coupled to a first input member 1116 and a second input member 1118. In some embodiments, the first input member 1116 and the second input member 1118 may be a gear. In other embodiments, the first input member 1116 and the second input member 1118 may be a pulley, a link, a lever arm, or could take other functional shapes. The first input member 1116 is pivotably coupled to ground 1120₁ via a first input rotational joint 1140 that provides at least one DoF with an axis of rotation defined by axis 1011. The second input member 1118 is pivotably coupled to ground 1120₂ via a second input rotational joint 1142 that provides at least one DoF with an axis of rotation defined by axis 1022. In certain embodiments, the first and second input rotational joints 1140, 1142 may be a 1 DoF rotation joint (e.g. pin joint, pivot joint, or revolute joint). In other embodiments, these input rotational joints may have other attributes that provide for two or three DoF.

In some examples, axes 1011 and 1022 may be approximately orthogonal. In other examples these axes may be at any two non-collinear axes in the plane defined by axes 1001 and 1002. For example, these axes may be at angles such as at a 70 degree angle or a 45 degrees angle or another angle. In certain embodiments, axis 1011 may be parallel to axis 1001, and axis 1022 may be parallel to axis 1001.

The first input member 1116 is configured to receive the first input I₁, which, in some embodiments, may be yaw rotation. The second input member 1118 is configured to receive the second input I₂, which, in some embodiments, may be pitch rotation. The first input member 1116 is coupled to the first swashplate 1124 via a first drive link 1144, thereby converting and transmitting the yaw rotation of first input member 1116 to a corresponding yaw rotation of the first swashplate 1124 about the first rotational joint R1. One end of the first drive link 1144 is pivotably coupled to the first input member 1116 via first pivot joint R11, and its other end is pivotably coupled to the first swashplate 1124 via a second pivot joint R12.

Similarly, the second input member 1118 is coupled to the first swashplate 1124 via a second drive link 1146, thereby converting and transmitting the pitch rotation of the second input member 1118 to a corresponding pitch rotation of the first swashplate 1124 about the first rotational joint R1. One end of the second drive link 1146 is pivotably coupled to the second input member 1118 via a third pivot joint R21, and its other end is pivotably coupled to the first swashplate 1124 via a fourth pivot joint R22. The first, second third, and fourth pivot joints R11, R12, R21, and R22 may be 2 DoF rotational joints (e.g. a universal or cardan joint) or a 3 DoF rotational joint (e.g. a spherical joint, ball and socket joint). In some embodiments, the pivot joints may have a flexure embodiment (e.g. via a notch flexure design, or an hourglass flexure design, or a beam flexure, etc.) In certain embodiments the third and fourth pivot joints R12 and R22 may be located on the first swashplate approximately 90 degrees apart with respect to the first rotational joint R1 and the center of rotation C1. In certain embodiments the centers of rotation of the third and fourth pivot joints R12 and R22 and the center of rotation C1 of the first rotational joint R1 all lie in the same plane as the plane formed by axes 1101 and 1102.

In certain embodiments, the first drive link 1144 may be the first input member 1116 itself without the need for separate members. In other words, the first input member and first drive link may be the same member or an extension of the other. Similarly, the second drive link 1146 and the second input member 1118 may be the same member or an extension of the other. In these embodiments, the first input I₁ and the second input I₂ may be directly received at the first drive link 1144 and second drive link 1146, respectively.

It is to be understood that sizes and locations of the first and second drive links 1144, 1146 and the first, second third, and fourth pivot joints R11, R21, R12, R22 can be chosen to achieve any desired ratio between the first input I₁ received at first input member 1116 and the resulting/corresponding rotation of the first swashplate 1124, and similarly between the second input I₂ received at the second input member 1118 and the resulting/corresponding rotation of the first swashplate 1124.

In some embodiments, there may be a correlation between the first input I₁ received at the first input member 1116 and the yaw rotation of the first swashplate 1124, independent of the second input I₂. There may also be a correlation between the second input I₂ received at the second input member 1118 and the pitch rotation of the first swashplate 1124, independent of the first input I₂. In other embodiments, some combination of the first input I₁ and the second input I₂ may produce yaw rotation of the first swashplate 1124, and some other combination of the first input I₁ and the second input I₂ received at the first and second input members 1116, 1118 may lead to pitch rotation of the first swashplate 1124.

In some embodiments and as illustrated in FIGS. 21A and 21B, the first input member 1116 along with first input link 1144 are may be analogous to the second input member 1118 and second input link 1146. In other embodiments, the first input member 1116 and the first input link 1144 may be a different combination of coupling to the second input member 1118 and the second input link 1146. In examples, the coupling between the input members and the first swashplate can include gears, belts, cables or ropes, chains, or other types of linkages (e.g. planar or spatial linkage mechanisms).

In embodiments and as illustrated in FIGS. 21A and 21B, the third input I₃ (e.g. roll rotation) is received at the third input member 1128 which may be coupled to the shaft 1130 via a friction drive 1032. In other embodiments, the third input I₃ may be transmitted from the third input member 1128 to the shaft 1130 via other transmissions such as via a cable, a belt, a gear or other transmission systems.

The third input member 1128 is pivotably coupled to ground 1120₃ via a third input rotational joint 1148 with an axis of rotation defined by axis 1033. In certain embodiments, the third input rotation joint 1148 may be a 1 DoF rotation joint (e.g. pin joint, pivot joint, or revolute joint). In some embodiments, axis 1033 may be parallel to axis 1003. In other embodiments, such as if the coupling includes a bevel gear drive between the third input member 1128 and the shaft 1130, axes 1033 and 1003 may not be parallel but instead be perpendicular or at another angle between 0 and 90 degrees. In some embodiments, the third input I₃ may be directly received at the shaft 1030 without the need for a third input member. In that case, the third input member and the shaft may be the same member or an extension of the other. For example, the shaft could be integrated with the rotor of an electric motor, or the shaft could be integrated with a dial or a lever that could be directly driven by a user.

The shaft 1130 is coupled to the ground 1120₄ via a third rotational joint R3 providing one DoF that allows rotation of the shaft 1130 with respect to ground 1120₄ about axis 1003. In some embodiments, the third rotational joint R3 may include rolling element bearings which allow roll rotation of the shaft 1130 about axis 1003, but does not allow articulation with respect to the ground 1120₄. In other embodiments, the third rotational joint R3 may include bushings, pins, or other bearing options to provide roll rotation. Axes 1301, 1302, and 1303 are fixed to the shaft 1030. The plane formed by axes 1301 and 1302 remains parallel to the plane formed by axes 1001 and 1002. In some embodiments, axis 1303 may be laterally offset from axis 1003. In other examples, axes 1303 and 1003 may be collinear.

As illustrated in FIGS. 21A and 21B, the second swashplate 1126 is coupled to the shaft 1130 via a roll drive coupling R4 that transmits roll rotation of the shaft 1130 (about axis 1003 or 1303) to a corresponding roll rotation of the second swashplate 1126 (about axis 1203) and while allowing for the articulation of the second swashplate 1126 with respect to the ground 1120₄ and/or the shaft 1130. The third rotational joint R3 prevents articulation rotation of the shaft 1030 with respect to the ground 1020₄. Depending on the articulation of the second swashplate 1126 with respect to ground 1020₄ (and therefore the shaft 1130), axis 1203 will generally not be collinear or parallel with axes 1003 or 1303, and instead can point at an angle with respect to axes 1003 and 1303.

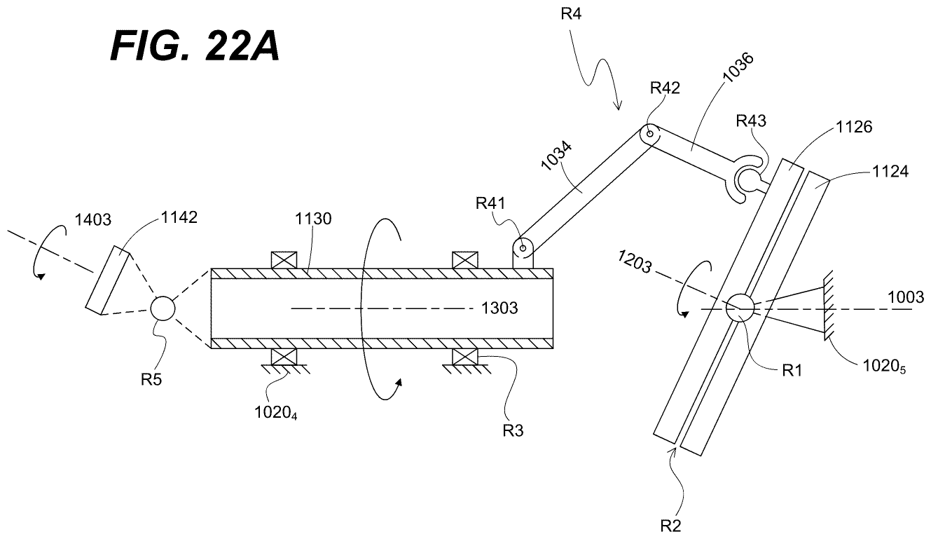

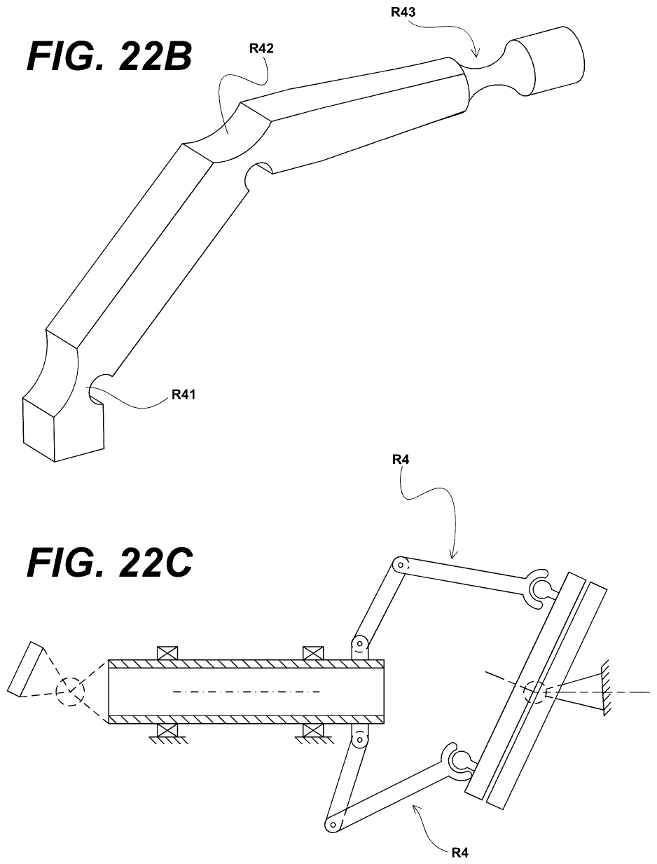

The roll drive coupling R4 may include multiple links and joints. In one embodiment as illustrated in FIGS. 21A and 21B, the roll drive coupling R4 includes a first rotational drive link joint R41 between the shaft 1130 and a roll drive coupling first link 1034, a second rotational drive link joint R42 between the roll drive coupling first link 1034 and a roll drive coupling second link 1036, and a third drive link rotational joint R43 between the roll drive coupling second link 1036 and the second swashplate 1126. The first and second rotational drive link joints R41 and R42 may be a 1 DoF rotational joint such as (e.g. pin joint, pivot joint, or revolute joint). The third rotational drink link joint R43 may be a two-DoF rotational joint (e.g. a universal joint, a cardan joint, a constant velocity (CV) joint, etc.) or a three-DoF rotational joint (e.g. spherical joint, ball and socket joint, etc.). In embodiments, any of these first, second, or third rotational drive link joints may have a flexure embodiment (e.g. via a notch flexure design, or an hourglass flexure design, or a beam flexure, or a wire flexure, or a notch flexure, etc.). One flexure based realization of the roll drive coupling R4 is shown in FIG. 22B, where R41 and R42 are flexure based living hinges that offer 1 rotational DoF while R43 is a flexure-based hourglass that offers 3 rotational DoF.

In some embodiments, the arrangement of links and rotational drive link joints in the roll drive coupling R4 may be referred to as a revolute(R)-revolute(R)-spherical(S) or simply R-R-S chain, listing the sequence of joints. In other embodiments, the roll drive coupling R4 may alternatively comprise a revolute(R)-revolute(R)-universal(U) or R-R-U chain. Other types of kinematic chains (i.e. arrangements of links and rotational joints) may also be used in the roll drive coupling R4. In further embodiments, more than one chain may be used at the same time for the roll-drive coupling R4. For example, two separate and independent R-R-S chains, spanning the shaft 1130 and the second swashplate 1126, may be employed to transmit the roll rotation of the shaft 1130 to a corresponding rotation of the second swashplate 1126 (see FIG. 22C). This can help increase the torque transmission capacity associated with roll rotation.

As illustrated in FIGS. 21A and 21B, the roll drive coupling R4 ensures that the third input I₃ (or roll rotation) received at the third input member 1128 and/or the shaft 1130 is transmitted to the second swashplate 1126, irrespective of the articulation of the second swashplate 1124. In other words, even though the shaft 1130 does not articulate with respect to ground 1020₅ while the second swashplate 1126 articulates with respect to ground 1020₅ (due to articulation of the first swashplate 1124), roll rotation of the shaft 1130 about axis 1303 (or effectively axis 1003) is coupled to the roll rotation of the second swashplate 1126 about axis 1203 (or effectively axis 1103). As shown, this roll drive coupling R4 arrangement provides roll rotation of the second swashplate 1126 that is exactly or approximately equal to the roll rotation of the shaft 1130. In other words, there is a 1:1 transmission ratio between the shaft 1130 and the second swashplate 1126. In practice, other transmission ratio of choice e.g. 1:2, 1:4, 2:1, or any other desired ratio can be achieved by appropriate choice of geometry, dimensions, and location of the various links, members and joints shown herewith, and/or via inclusion of additional linkage(s), belt(s), pulley(s), rope(s), gear(s), or other transmission elements. In another embodiment, the roll drive coupling R4 may be used to couple roll rotation directly between the third input member 1128 and the second swashplate 1126.

The shaft 1130 is coupled to an output member 1142 (e.g. a distal manipulator) via a first output rotational joint R5 that transmits roll rotation of the shaft 1130 (about axis 1003 and 1303) to roll rotation of the distal manipulator 1142 while allowing for the articulation of the distal manipulator with respect to the shaft 1142 about a center of rotation C5.

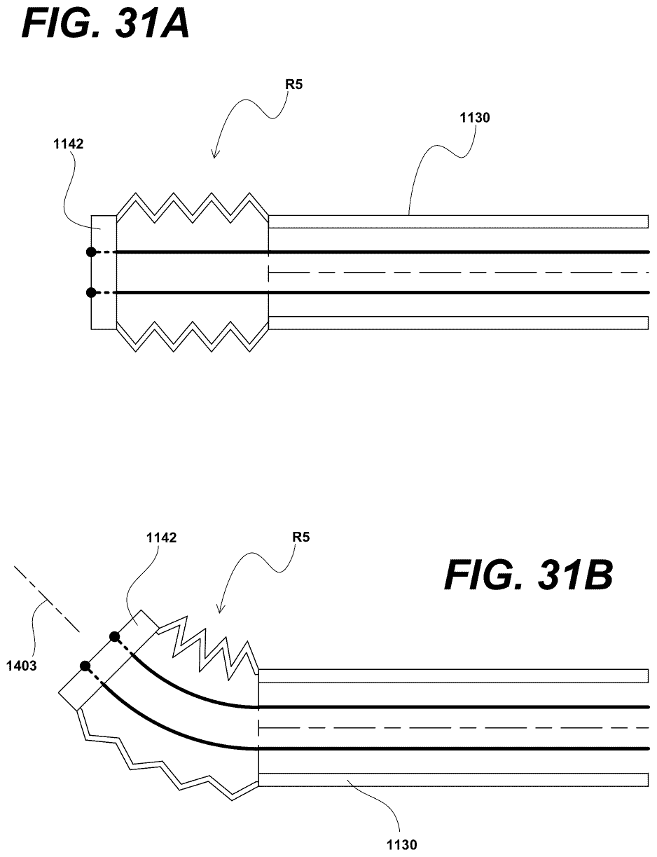

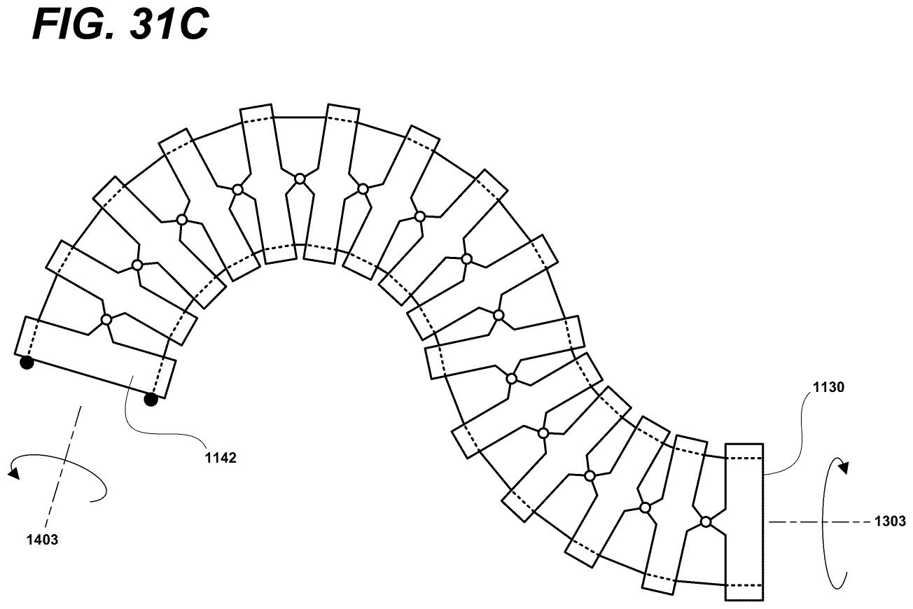

In one embodiment, the first output rotational joint R5 is a two DoF joint that allows articulation (pitch and yaw rotations) between the distal manipulator 1142 and the shaft 1130 and transmits roll rotation. The first output rotational joint R5 may be a universal joint, a cardan joint, a constant velocity (CV) joint, or another joint that provides two DoF. In other embodiments the first output rotational joint R5 may also be a series of universal or cardan joints, which together accomplish allowing articulation (pitch and roll rotations) between the distal manipulator 1142 and shaft 1130 and transmit roll rotation. A series of universal joints at the first output rotational joint R5 can enable the distal manipulator to take a complex serpentine shape in the pitch and yaw directions (as shown in FIG. 31C).

Axes 1401, 1402, and 1403 are fixed to the distal manipulator 1142. In the nominal (or non-articulated) configuration as shown in FIGS. 21A and 21B, axis 1403 is aligned and collinear with axes 1003 and 1303. Axes 1401 and 1402 form a plane that is parallel to the plane formed by axis 1301 and 1302. Upon articulation (for example shown in FIG. 23), the distal manipulator 1142 and axis 1403 points at an angle with respect to axis 1303. Axes 1401 and 1301 can be parallel, and axes 1402 and 1302 can be parallel when the distal manipulator 1142 is non-articulated.

Referring now to FIG. 22A, the first output rotational joint R5 provides roll rotation of the shaft 1130 about axis 1303, which is transmitted to roll rotation of the distal manipulator 1142 about axis 1403, irrespective of the articulation of the distal manipulator 1142. In other words, even though the shaft 1130 does not articulate with respect to ground 1020₅, the distal manipulator 1142 articulates with respect to the shaft 1130 and the ground 1020₅ due to articulation of the second swashplate 1126, and the roll rotation of the shaft 1130 about axis 1303 is coupled to the roll rotation of the distal manipulator 1142 about axis 1403.

The roll drive coupling R4 between the shaft 1130 and the second swashplate 1126, and the first output rotational joint R5 between the shaft 1130 and the distal manipulator 1142 ensure that the roll rotation of the second swashplate 1126 about axis 1203, the roll rotation of the shaft 1130 about axis 1303, and the roll rotation of the distal manipulator 1142 about axis 1403 are all coupled to each other. All three of these roll rotations happen together in synchronization, which is shown in FIGS. 23-26, illustrating multiple positions in a rotational cycle.

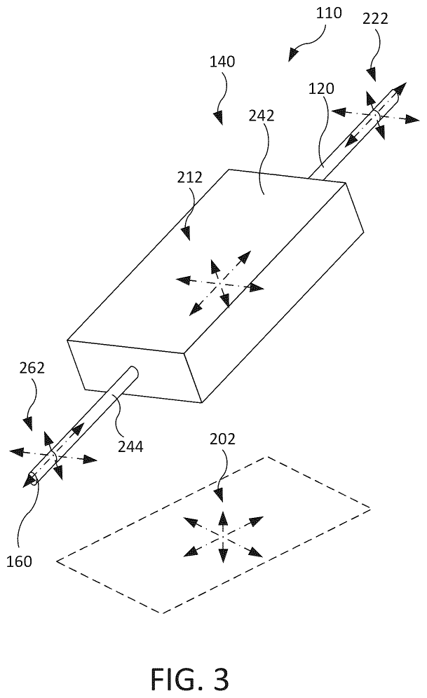

The distal manipulator 1142 is coupled to the second swashplate 1126 via an “articulation output transmission” that transmits the articulation of the second swashplate 1126 to articulation of the distal manipulator 1142. As illustrated in FIG. 21A and FIGS. 3-6, the articulation output transmission includes four cables (1601, 1602, 1603, and 1604). One end of each cable 1601, 1602, 1603, 1604 is coupled to the second swashplate 1126, while the other end of each cable 1601, 1602, 1603, 1604 is connected to the distal manipulator 1142. The cables 1601, 1602, 1603, 1604 are capable of transmitting tension (i.e. can be pulled but cannot be pushed). Two cables transmit rotation in each articulation rotation. For example, a first pair of cables 1601 and 1603 transmit pitch rotation in positive and negative directions, while a second pair of cables 1602 and 1604 transmit yaw rotation in positive and negative directions.

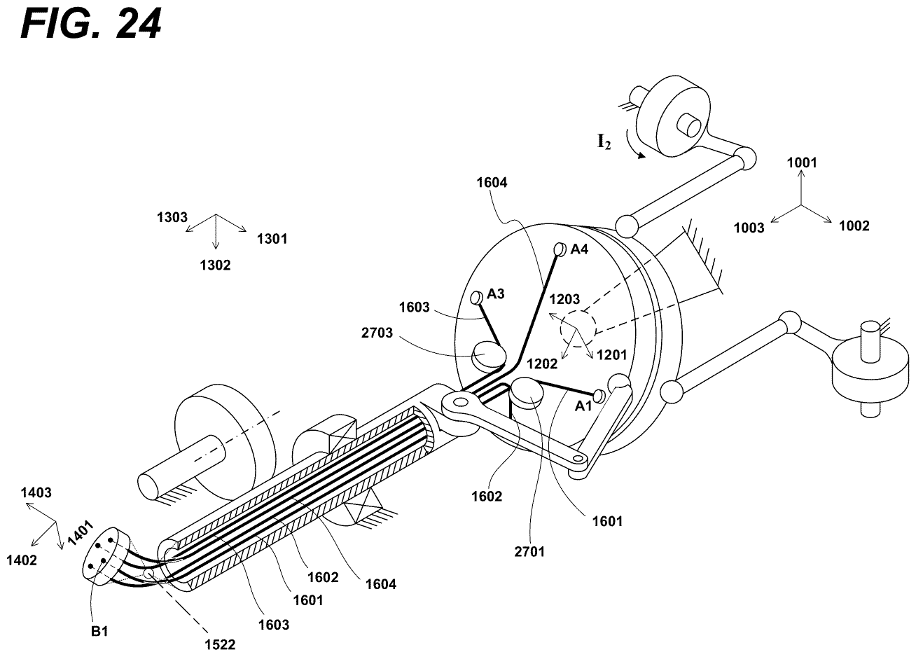

In some embodiments, the cables 1601, 1602, 1603, 1604 are shown to be routed through the shaft 1130 between the second swashplate 1126 and the distal manipulator 1142. In other embodiments, the cables 1601, 1602, 1603, 1604 may be routed via different paths within or outside the shaft 1130, or completely independent of the shaft 1130. In some embodiments, redirect pulleys 2701, 2702, 2703, 2704 are provided to route and direct the cables 1601, 1602, 1603, 1604. Between the second swashplate 1126 and entry location at the proximal end of the shaft 1130, there is at least one redirect pulley 2701, 2702, 2703, 2704 per cable 1601, 1602, 1603, 1604. FIG. 23 illustrates pulley 2702 associated with cable 1602 and pulley 2704 associated with cable 1604. FIG. 24 illustrates pulley 2701 associated with cable 1601 and pulley 2703 associated with cable 1603. These redirect pulleys 2701, 2702, 2703, 2704 are mounted to the shaft (generally via a pin joint), such that as the shaft rotates about axis 1303, the pulleys also orbit about axis 1303, along with the shaft. It is understood that the illustrated figures only show two pulleys for clarity of the images, and the illustrated embodiment as described includes four pulleys 2701, 2702, 2703, 2704, each associated with their respective cable 1601, 1602, 1603, 1604. In other examples, multiple redirect pulleys or pins or idlers or riding surfaces, or other common components/features may be used to suitably direct the cables. In some instances, there may be no redirect pulleys.

In one embodiment, a first connection point A1, A2, A3, A4 of a given cable 1601, 1602, 1603, 1604 on the second swashplate 1126 may have a corresponding second connection point B1, B2, B3, B4 at an analogous location on the distal manipulator 1142. For example as illustrated in FIG. 23, looking from the distal end to the proximal end, one end of cable 1601 may be coupled to the second swashplate 1126 at or close to the 12 o'clock location at the first connection point A1, while the other end of cable 1601 may be coupled to the distal manipulator 1142 also at or close to the 12 o'clock location at the second connection point B1. Similarly, the two ends of cable 1602 may be coupled to the second swashplate 1126 (at connection point A2) as well as to the distal manipulator 1142 (at connection point B2) at their respective 3 o'clock (approximately) locations, the two ends of cable 1603 may be coupled to both the second swashplate 1126 (at connection point A3) as well as to the distal manipulator 1142 (at connection point B3) at their respective 6 o'clock (approximately) locations, and the two ends of cable 1604 may be coupled to both the second swashplate 1126 (at connection point A4) as well as to the distal manipulator 1142 (at connection point B4) at their respective 9 o'clock (approximately) locations. This arrangement of cables 1601, 1602, 1603, 1604 results in an operation such that when the second swashplate 1126 is articulated upward (i.e. positive pitch rotation), the distal manipulator 1142 is also articulated upward (i.e. positive pitch rotation). When the second swashplate 1126 is articulated downward (i.e. negative pitch rotation), the distal manipulation 1142 is also articulated downward (i.e. negative pitch rotation). When the second swashplate 1126 is articulated rightward (i.e. negative yaw rotation), the distal manipulation 1142 is also articulated rightward (i.e. negative yaw rotation). When the second swashplate 1126 is articulated leftward (i.e. positive yaw rotation), the distal manipulation 1142 is also articulated leftward (i.e. positive yaw rotation).

In other embodiments, the connection points for the cables 1601, 1602, 1603, 1604 at their respective first connection points A1, A2, A3, A4 and their respective second connection points B1, B2, B3, B4 may be different. For example, one end of cable 1601 may be coupled to the second swashplate 1126 at or close to the 12 o'clock location, while the other end of cable 1601 may be coupled to the distal manipulator 1142 at (or close to) the 6 o'clock location. In this case an upward articulation of the second swashplate 1126 (i.e. positive pitch rotation) may lead to a downward articulation of the distal manipulator 1142 (i.e. negative pitch rotation). In yet another alternative arrangement, one end of cable 1601 may be coupled to the second swashplate 1126 at or close to the 12 o'clock location, while the other end of cable 1601 may be coupled to the distal manipulator 1142 at (or close to) the 3 o'clock location. In this case an upward articulation of the second swashplate 1126 (positive pitch rotation) may lead to a rightward articulation of the distal manipulator 1126 (negative yaw rotation).

Furthermore, the radial location of the first connection points A1, A2, A3, A4 where the cable end is coupled to the second swashplate 1126 (i.e. distance of cable coupling point A1/A2/A3/A4 from center C1) relative to the radial location the second connection points B1, B2, B3, B4 of where the other cable end is coupled to the distal manipulator 1142 (i.e. distance of cable coupling point B1/B2/B3/B4 from center C5) may determine the ratio between articulation angle of the second swashplate 1126 and the distal manipulator 1142. By changing or optimizing these radial locations of cable coupling locations on the second swashplate 1126 and/or the distal manipulator 1142, a desirable transmission ratio between the articulation (i.e. yaw and/or pitch rotations) of the second swashplate 1126 and the articulation of the distal manipulator 1142 may be achieved. Furthermore, one can implement and achieve different transmission ratios for the yaw rotation compared to the pitch rotation.

In some embodiments, the cables ends may be coupled to the second swashplate 1126 or the distal manipulator 1142 via crimping the cables ends in place with a ball crimp. In other embodiments, the cable ends may be crimped in place via other crimps or by clamped in place. Crimps used on the cable can have various shapes including, but not limited to, ball crimps or cylindrical crimps. The coupling could also include the use of a ball interface (e.g. a ball and socket or spherical joint) or a universal-joint interface that allows the cable end to swivel freely with respect to the second swashplate 1126 or the distal manipulator 1142, while still effectively transmitting tension.

In one preferred arrangement, all four first connection points A1, A2, A3, A4 of the four cables 1601, 1602, 1603, 1604 on the second swashplate 1126 are in the same plane, which also passes through the center of rotation C1 of the first rotational joint R1.

Thus, the distal manipulator 1142 receives articulation rotation from the second swashplate 1126 and roll rotation from the shaft 1130. However, since the roll rotation of the second swashplate 1126, the shaft 1130, and the distal manipulator 1142 about their respective roll axes (1203, 1303, and 1403, respectively) are synchronized, the cables 1601, 1602, 1603, 1604 maintain their relative lateral/circumferential positions with respect to these three members (second swashplate 1126, shaft 1130, and distal manipulator 1142) without getting twisted up during roll rotation. Although four cables are shown in the FIGS. 23-26, in practice three cables, five cables, or more cables could be used to transmit pitch and yaw articulation from the second swashplate to the distal manipulator.

Referring now to FIGS. 23-26, operation of the transmission system 1110, and in particular the roll rotation of the transmission system 1110 is illustrated. The transmission system 1110 is configured to receive multiple separate and independent inputs including articulation input and roll input. Articulation input (i.e. the first input I₁ and the second input I₂) may include yaw input and pitch input. Yaw input and pitch input may be received separately and independently at the first and second input members 1116, 1118. The roll input (the third input I₃) may be received at the third input member 1128. In embodiments, the first input I₁, the second input I₂, and the third input I₃ are received independently at three different input members 1116, 1118, 1128 and delivered together to the single output member—the distal manipulator 1142.

The multiple inputs such as the first input I₁ (i.e. yaw input), the second input I₂ (i.e. pitch input), and the third input I₃ (i.e. roll input) are all received at their respective input members (e.g. first input member 1116, second input member 1118, and third input member 1126, respectively) entirely independent of each other. This means that any of the three inputs I₁, I₂, I₃ may be received by driving their respective input member 1116, 1118, 1126, irrespective of the status of the other two inputs or input members.

The construction of the transmission system 1110 is such that whatever articulation input (i.e. yaw and/or pitch) is received at the first swashplate 1124 is transmitted to the second swashplate 1126 irrespective of any roll rotation of the second swashplate 1126 about axis 1103 (or equivalently 1203).

Furthermore, the articulation input sub-system 1111 is such that whatever yaw rotation received at the first input member 1116 is transmitted to the first swashplate 1124 and then to the second swashplate 1126 (yaw rotation about axis 1101) irrespective of any pitch rotation received at the second input member 1118 and any roll rotation of the second swashplate 1126 about axis 1103 (or equivalently 1203). Similarly, whatever pitch rotation received at the second input member 1118 is transmitted to the first swashplate 1124 and then to the second swashplate 1126 (pitch rotation about axis a102) irrespective of any yaw rotation received at the first input member 1116 or any roll rotation of the second swashplate 1126 about axis 1103 (or equivalently 1203). Furthermore, any roll rotation input received at the third input member 1128 is transmitted to the second swashplate 1126 via the roll drive coupling R4 irrespective of any articulation of the second swashplate 1126 about axes 1101 and/or 1102.

Even though the first swashplate 1124 does not have a roll rotation while the second swashplate 1126 can experience roll rotation when a roll rotation input is provided, the two swashplates 1124, 1126 remain coupled via the second rotational joint R2, which ensures that both swashplates 1124, 1126 articulate together (about axes 1101 and/or 1102) while remaining free to have a relative roll rotation about axis 1103 (or equivalently 1203). The kinematic arrangement of joints and interfaces is such that there is no jamming or over-constraint or conflict between the two swashplates 1124, 1126. Similarly, there is no kinematic conflict or back-driving between the inputs 1116, 1118, 1128. Any one input member 1116, 1118, 1128 may be driven (i.e. receive its respective input) independent of the status of the other two inputs. As described above, the second swashplate 1126 independently receives articulation input from the first swashplate 1124 and roll input from the shaft 1130, and exhibits a combination of all three rotations: articulation (including yaw and pitch) and roll.

The transmission system 1110 further transmits these three rotations to the distal manipulator 1142. The roll input received at the third input member 1128 is transmitted to the distal manipulator 1142 via the shaft 1130 and the first output rotational joint R5. The articulation of the second swashplate 1126 is transmitted to the distal manipulator 1142 via an articulation output transmission that may include the plurality of cables 1601, 1602, 1603 and 1604.

The kinematic arrangement of the roll input sub-system 1113 and the output sub-system 1114 ensures that the second swashplate 1126, the shaft 1130, and the distal manipulator 1142 all rotate in synchrony about their respective roll axes, 1203, 1303, and 1403, irrespective and independent of any articulation inputs transmitted through the transmission system 1110. The articulation inputs determine the articulation angle of axis 1203 (on the second swashplate 1126) with respect to axis 1003, and the articulation angle of axis 1403 (on the distal manipulator 1142) with respect to axis 1303, but do not impact the roll rotation of the second swashplate 1126 about axis 1203 and corresponding roll rotation of the distal manipulator 1142 about axis 1403.

Similarly, the articulation inputs received at the first swashplate 1124 are transmitted to the second swashplate 1126 via the second rotational joint R2 and further to the distal manipulator 1142 via the articulation output transmission comprising four cables 1601, 1602, 1603, 1604. This articulation transmission happens irrespective of the roll rotation of the second swashplate 1126, the shaft 1130, and the distal manipulator 1142, and is made possible via the roll decoupling (i.e. rotational DoF about the roll axis 1103) between the second swashplate 1126 and the first swashplate 1124 offered by the second rotational joint R2.

Thus, three rotational inputs I₁, I₂, I₃ are received separately and independently at three different locations at three different input members 1116, 1118, 1128 in the transmission system 1110 and do not impact or conflict with or constrain each other in any way; however, the transmission system 1110 transmits and delivers all three of these rotations altogether at a single output member—the distal manipulator 1142.

The construction of transmission system 1110, as described above, ensures that any given input is transmitted independently to the distal manipulator 1142. For example, yaw rotation input (i.e., the first input I₁) is transmitted to the distal manipulator 1142 as a corresponding yaw rotation output O₁ based on a ratio determined by various geometries and dimensions chosen as part of the construction, but this mapping from yaw rotation input I₁ to yaw rotation output O₁ is independent of any pitch rotation or roll rotation transmitted via the transmission. This attribute is referred to as fidelity of transmission or when a particular input motion is faithfully (e.g. without major losses or corruption or distortion) is transmitted to a corresponding output motion irrespective of whatever other motions are being transmitted via the transmission system.

Upon receiving a roll input (i.e. the third input I₃) at the third input member 1128, the distal manipulator 1142 faithfully exhibits a corresponding roll rotation about axis 1403, completely irrespective of which orientation the distal manipulator 1142 is articulated in (in response to articulation inputs (i.e. I₁ and I₂) received at first and/or second input members 1116, 1118). In fact, this articulation may be fixed by holding the articulation inputs steady in any desired positions, or this articulation could be adjusted continuously or intermittently by dynamically varying the articulation inputs at the first and/or second input members 1116, 1118. Irrespective of that, the mapping of the roll input about axis 1033 and shaft 1130 about axis 1303 to roll rotation of the distal manipulator 1142 about axis 1403 is preserved. This is a notable attribute of the transmission mechanism presented herewith.

Similarly, the transmission system faithfully transmits yaw rotation I₁ from the first input member 1116 (rotated about axis 1011 with respect to ground 10201) to a corresponding rotation of the distal manipulator 1142 (e.g. a yaw rotation O₁), at a ratio determined by the geometry and dimensions of certain members and joints in the transmission system 1110, irrespective of any roll rotation that is received at the third input member 1128 and transmitted via the shaft 1030 and the first 5 output rotational joint R5 to the distal manipulator 1142. Furthermore, this yaw rotation of the distal manipulator 1142 happens about an axis 1511 that is close to the center of rotation C5 and that remains approximately parallel to axis 1001 even as the distal manipulator 1142 (and axis 1401 attached to the distal manipulator 1142) rotates in roll about axis 1403. Thus, if the yaw rotation input about axis 1011 (as applied to the first input member 1116 with respect to ground 10201) is held steady, the distal manipulator 1142 articulates in yaw rotation with respect to ground 1020₄ about an axis 1511 approximately parallel to 1001, and maintains a steady articulation orientation even as the distal manipulator 1142 rotates in roll about axis 1403.

Similarly, the transmission system 1110 faithfully transmits pitch rotation from the second input member 1118 (rotated about axis 1022 with respect to ground) to a corresponding rotation of the distal manipulator 1142 (e.g. a pitch rotation), at a ratio determined by the geometry and dimensions of certain members and joints in the transmission system, irrespective of any roll rotation that is received at the third input member 1128 and transmitted via the shaft 1030 and the output rotational joint R5 to the distal manipulator 1142. Furthermore, this pitch rotation of the distal manipulator 1142 happens about an axis 1522 that is close to the center of rotation C5 and that remains approximately parallel to axis 1002 even as the distal manipulator 1142 (and axis 1402 attached to the distal manipulator 1142) rotates in roll about axis 1403. Thus, if the pitch rotation input about axis 1022 (as applied to the second input member 1118 with respect to ground) is held steady, the distal manipulator 1142 articulates in pitch rotation with respect to ground about an axis 1522 approximately parallel to 1002, and maintains a steady articulation orientation even as the distal manipulator 1142 rotates in roll about axis 1403.

Similarly, the transmission system 1110 faithfully transmits any combination of yaw and pitch rotations from the first and second input members 1116, 1118 to a corresponding articulation of the distal manipulator 1142 (combination of yaw and pitch rotations), irrespective of any roll rotation that is received at the third input member 1128 and transmitted via the shaft 1130 and the first output rotational joint R5 to the distal manipulator 1142. This articulation of the distal manipulator 1142 happens about an axis that remains steady with respect to the ground, even as the distal manipulator 1142 and axes 1401 and 1402 attached to the distal manipulator 1142 rotates in roll about axis 1403.

This is further evident in FIGS. 23 through 26. Each of these figures shows the first input I₁ (pitch rotation input) provided and held at the first input element 1116 that results in the first swashplate 1124 to articulate about axis 1102 (e.g. in pitch rotation) with respect to the center of rotation C1 of the first rotational joint R1. This articulation of the first swashplate 1124 is transmitted to the second swashplate 1126 which also exhibits a pitch rotation about axis 1102 with respect to the center of rotation C1. This causes the axis 1203 (attached to the second swashplate 1126) to be pointed at an articulated angle relative to axis 1003. The articulation of the second swashplate 1126 (i.e. pitch rotation) is transmitted via articulation output transmission (an arrangement of cables 1601, 1602, 1603, and 1604), to a corresponding articulation (pitch rotation) of the distal manipulator 1142 with respect to the shaft 1130 about axis 1522, that is parallel to axis 1002, passing through the center of rotation C5. The distal manipulator 1142 is held steady in the articulated condition, resulting in axis 1403 (fixed to the distal manipulator 1142) also pointing at an articulated angle with respect to axis 1303.

At the same time a roll rotation input (i.e. the third input I₃) received at the third input member 1128 causes the shaft 1130 to rotate about axis 1303, and this roll rotation is transmitted via the roll drive coupling R4 to the second swashplate 1126 causing the latter to rotate about axis 1203. This roll rotation input is also transmitted from the shaft 10130 to the distal manipulator 1142, via the first output rotational joint R5, causing the distal manipulator 1142 to rotate about axis 1403. This also results in axes 1401 and 1402, which are fixed to the distal manipulator 1142, to rotate about axis 1403.

Referring to FIGS. 23-26, the figures show four time instances of the transmission system 1110, where the second input I₂ (pitch rotation) is driven and held fixed, the first input I₁ (yaw rotation) is held fixed in its nominal/neutral position, and the third input I₃ (rotation rotation) causes the shaft 1130 to rotate. FIGS. 23-26 illustrate four time instances of the transmission system as the shaft 1130 rotates in quarter turn increments throughout one rotational cycle of the shaft 1130.

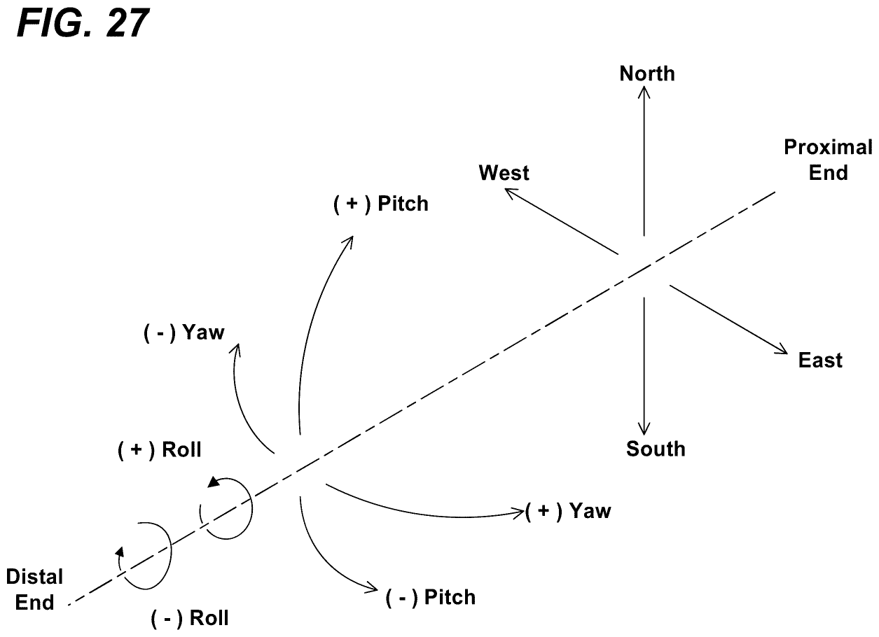

Referring to FIG. 23, axes 1301, 1302, and 1303 are fixed to the shaft 1030. Even though axis 1303 is shown to be laterally offset from axes 1003, axes 1303 and 1003 can be collinear, while the plane formed by axes 1301 and 1302 remains parallel to the plane formed by axes 1001 and 1002. Axes 1203 points normal to the second swashplate 1126 in an articulated direction with respect to axis ground frame axis 1003. Axis 1403 (fixed to the distal manipulator 1142) also points at an articulated angle with respect to axis 1303. Axis 1201 (fixed to the second swashplate 1126), axis 1301 (fixed to the shaft 1030), and axis 1401 (fixed to the distal manipulator 1142) all point towards north. References “north, south, east, and west” for directions with respect to the transmission system 1110, are illustrated in FIG. 27.

Referring to FIG. 24, since the second input I₂ (pitch rotation) is held steady, axes 1203 and 1403 also remain in the same respective articulated orientation as in FIG. 23. However, the roll drive coupling R4, the second swashplate 1126, the shaft 1130, and the distal manipulator 1142 have all rotated in roll by about a quarter turn compared to the position in FIG. 23. As a result, axis 1201 (fixed to the second swashplate 1126), axis 1301 (fixed to the shaft 1130), and axis 1401 (fixed to the distal manipulator 1142) now all point towards east.

It can be seen that while cable 1601 is in the north position in FIG. 23, by the time the transmission gets to the instance illustrated in FIG. 24, this north position is taken by cable 1604 while cable 1601 moves to the east position. All this while, the articulation of the second swashplate 1126 is held steady. The drive coupling R4 is now in a new position (pointing east) and adjusts to accommodate the fact the distance between R41 and R43 is now shorter.

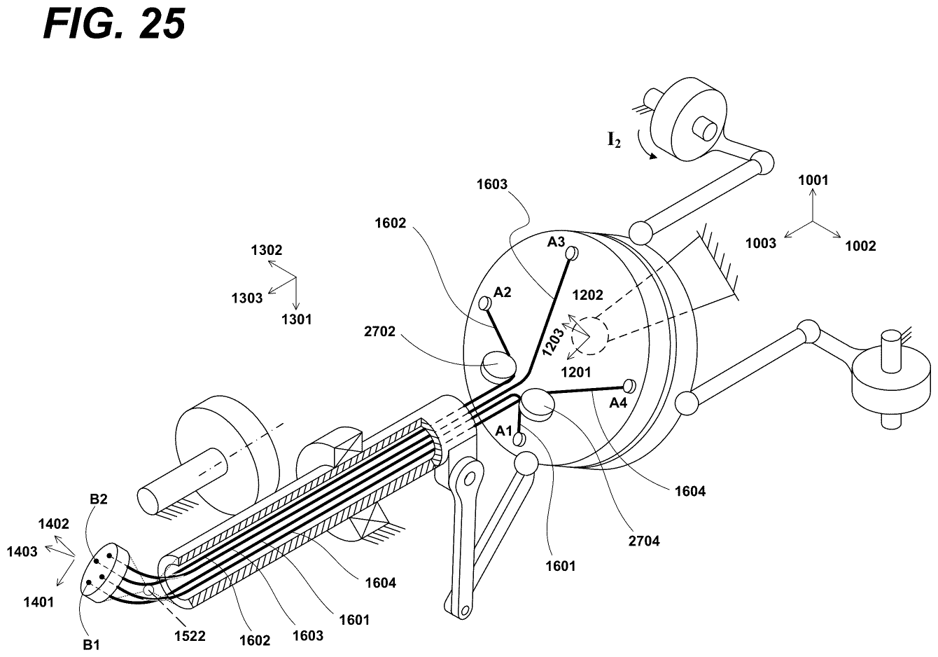

Referring to FIG. 25, since the second input I₂ (pitch rotation) is still held steady, axes 1203 and 1403 also remain in the same respective articulated orientation as in FIG. 24. However, the roll drive coupling R4, second swashplate 1126, the shaft 1130, and the distal manipulator 1142 have all rotated in roll by about another quarter turn compared to FIG. 24. As a result, axis 1201 (fixed to the second swashplate 1126), axis 1301 (fixed to the shaft 1130), and axis 1401 (fixed to the distal manipulator 1128) now all point towards south.

It can be seen that while cable 1604 is in the north position in FIG. 24, by the time the transmission gets to the instance illustrated in FIG. 25, this north position is taken by cable 1603 while cable 1604 moves to the east position. All this while, the articulation of the second swashplate 1126 is held steady. The drive coupling R4 is now in a new position (pointing south) and further adjusts to accommodate the fact the distance between R41 and R43 is now further reduced.

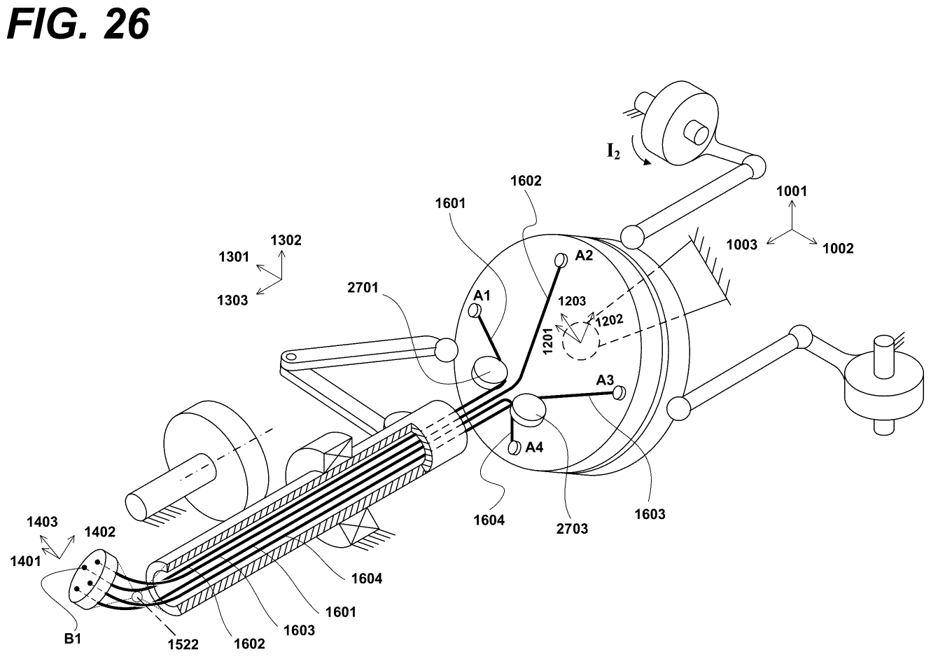

Referring to FIG. 26, since the second input I₂ (pitch rotation) is still held steady, axes 1203 and 1403 also remain in the same respective articulated orientation as in FIG. 25. But the roll drive coupling R4, second swashplate 1126, the shaft 1130, and the distal manipulator 1142 have all rotated in roll by yet another quarter turn compared to FIG. 25. As a result, axis 1201 (fixed to the second swashplate 1126), axis 1301 (fixed to the shaft 1130), and axis 1401 (fixed to the distal manipulator 1142) now all point towards west.

It can be seen that while cable 1603 is in the north position in FIG. 25, by the time the transmission gets to the instance illustrated in FIG. 26, this north position is taken by cable 1602 while cable 1603 moves to the east position. All this while, the articulation of the second swashplate 1126 is held steady. The drive coupling R4 is now in a new position (pointing west) and adjusts to accommodate the fact the distance between R41 and R43 has now increased compared to the previous time instance illustrated in FIG. 25.

The instances illustrated in FIGS. 23-26 show that the second swashplate 1126, shaft 1130, and distal manipulator 1142 all rotate in roll in a synchronized manner because of the roll drive coupling R4 and the first output rotational joint R5, even as the second swashplate 1126 and the distal manipulator 1142 are articulated. Furthermore, since the redirect pulleys 2701, 2702, 2703, and 2704 are mounted on the shaft 1130 (i.e. the respective pins or axes of rotation of these pulleys are coupled to the shaft), these pulleys 2701, 2702, 2703, and 2704 also orbit around axis 1303 as the shaft 1130 rotates (in roll) about axis 1303. Synchronization of roll across these multiple members (second swashplate 1126, shaft 1130, and distal manipulator 1142) on the output side of the transmission system 1110 independent of any articulation (pitch and/or yaw rotation) ensures that cables 1601, 1602, 1603 and 1604 all remain in their relative location laterally/circumferentially with respect to these members. In fact, the roll drive coupling R4 and the output articulation transmission (comprising cables 1601, 1602, 1603 and 1604) also rotate in synchronization with these members about the axis 1303. This ensures that the cables do not get twisted or wound up, and do not interfere with the roll drive coupling R4 in the presence of roll rotation being transmitted from the third input member 1128 to the distal manipulator 1142. This provides the transmission with the ability to transmit roll rotation to the distal manipulator 1142 in positive or negative directions without any physical limits or constraints on the range of roll rotation imposed by any member of the transmission system.

A key attribute of this transmission system 1110 is its high torque transmission capacity in roll rotation. In the preferred embodiment shown, the roll input received at the third input member 1128 is transmitted to the shaft 1130 and then, via the roll drive coupling R4, to the second swashplate 1126 and via the first output rotational joint R5 to the distal manipulator 1142. All these roll transmission members, elements, joints, and interfaces are such that relatively large amounts of forces and torques can be transmitted via them to the distal manipulator 1142. This provides a high torque capability in this transmission system 1110, particularly in the roll rotation about axis 1403.

Typically, it is common to achieve high torque transmission capabilities in yaw and/or pitch rotation directions because of the transmission elements involved. However, roll is typically transmitted via an internal roll transfer member that runs through the center of the transmission system or shaft to the distal manipulator. This roll transfer member is flexible in bending to allow for the articulation at the input of the transmission and articulation of the distal manipulator. But this flexibility as well as the geometry of such a flexible member limits its torque transmission capacity in the roll direction. Furthermore, the geometry of such a roll transfer member also limits how tight the articulation of the distal manipulator can be (i.e. how small the radius of curvature achieved by the rotational joint R5). Tight articulation requires the member to be thinner so that it can flex or take a tight bend more easily, but this in turn further limits the torque transfer capability.

Several variations of the various sub-systems of transmission system 1110 are possible.

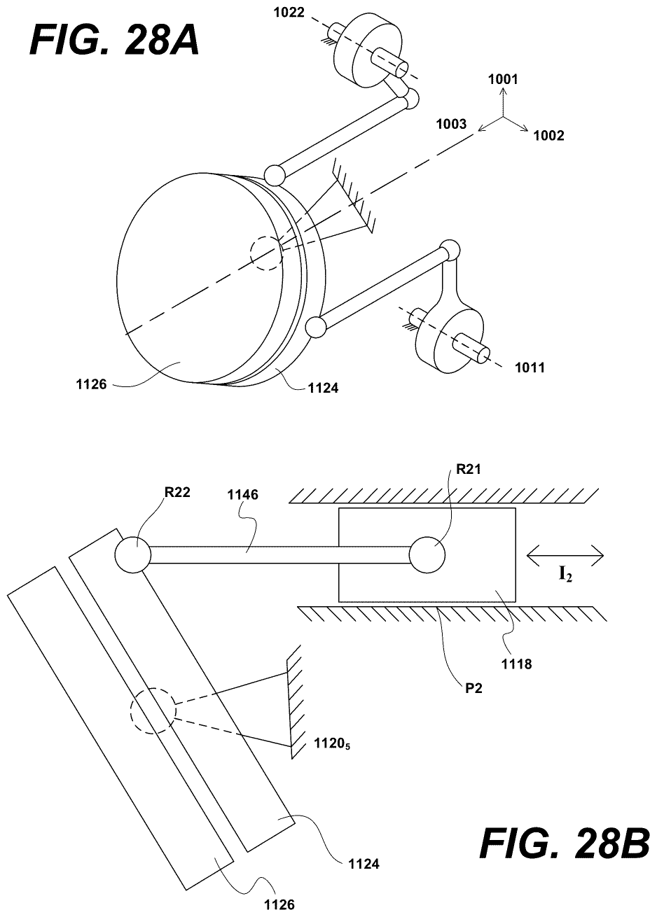

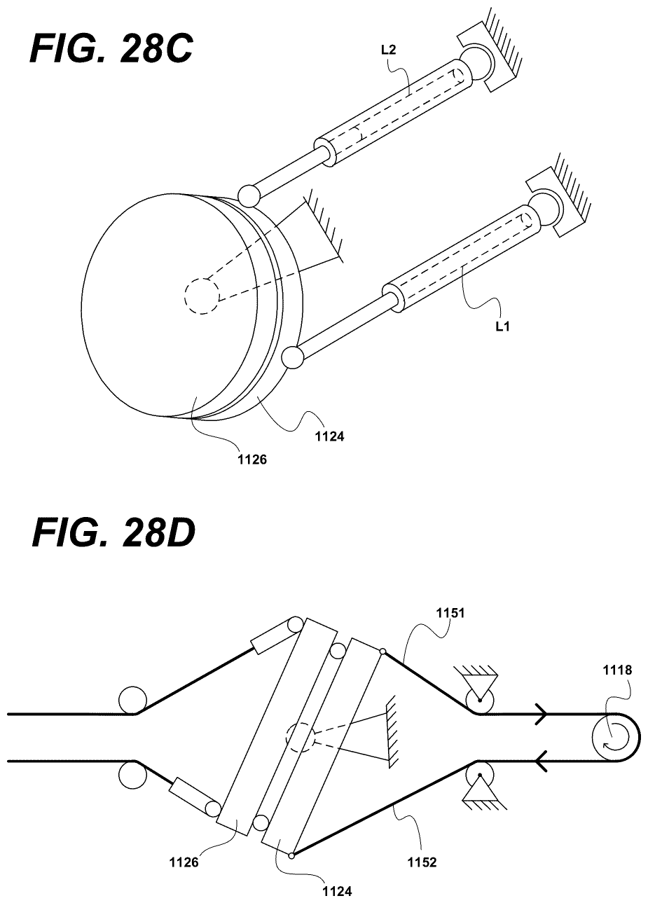

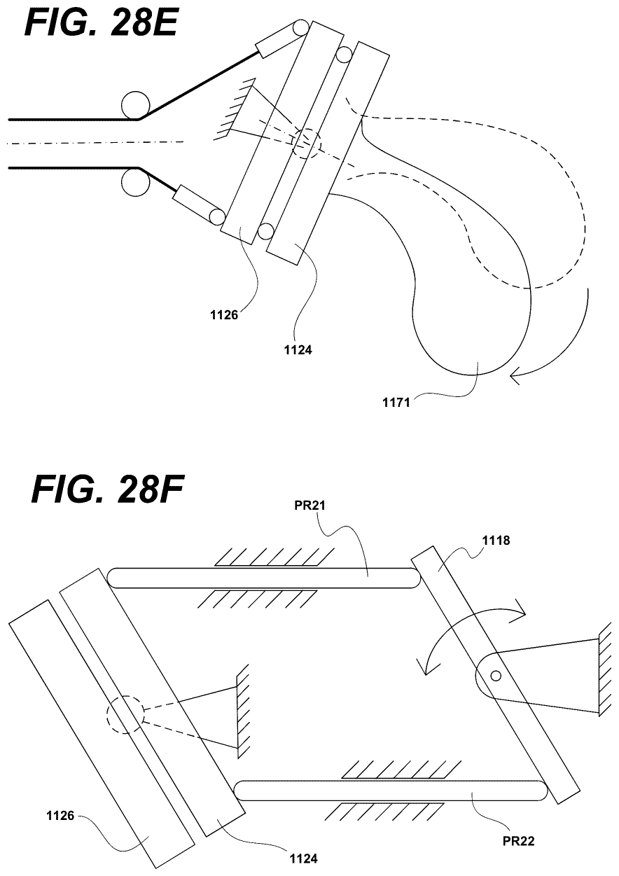

Referring to FIG. 21A, first and second input members 1116, 1118 of the articulation input sub-system 1111 are shown to be pivotably mounted to the ground 1120₁, 1120₂ via respective revolute joints that have axes 1011 and 1022. While these axes are shown to be approximately perpendicular, in alternate embodiments, these axes could be parallel to each other and in the direction of axis 1001 or axis 1002. Additionally in FIG. 21A, the first input member 1116 is shown as a lever arm or link pivoting about axis 1011 in a plane formed by axes 1002 and 1003. In other embodiments, axis 1011 could be parallel to axis 1002 and the first input member 1116 could be pivoting in a plane formed by axes 1001 and 1003 as shown in FIG. 28A.

Referring to FIG. 28B, an embodiment is illustrated with the second input member as a slider 1118 that is coupled to ground 1120₅ via a prismatic joint P2. A first revolute joint R21 connects the first end of the second drive link 1146 to the slider 1118, and a second revolute joint R22 connects the first end of the second drive link 1146 to the slider 1118.

In general, the first input member 1116 and second input member 1118 can take many different geometries and configurations, as long as the first end (R11) of the first drive link 1144 and first end (R21) of the second drive link 1146 are driven approximately along a direction parallel to axis 1003.

Referring to FIG. 28C, an embodiment is illustrated that shows another variation of the articulation input sub-system 1111, wherein the first input I₁ and the second input I₂ are provided to the first swashplate 1124 via a first linearly actuated link L1 and a second linearly actuated link L2. These linearly actuated links L1, L2 could be piston-cylinders or some other linear actuator such as linear motor, or rotator motor with a lead screw, or a voice coil actuator, or a piezoelectric actuator etc. One end of each linearly actuated link is coupled to ground via a 2-DoF or 3-DoF rotational joint, and the other end of each linearly actuated link is coupled the first swashplate 1124 via another 2-DoF or 3-DoF joint.

In yet another embodiment shown in FIG. 28D, the articulation input transmission 1111 could comprise a set of cables. A first pair of cables (1151 and 1152) may rotate the first swashplate 1124 in pitch rotation. The first pair of cables 1151, 1152 may be driven by a second input member 1118, which is a pulley. Although not shown, a second pair of cables may be driven by a first input member (i.e. another pulley) that can be used to rotate the first swash plate 1124 in yaw rotation.

Although in most instances here, the input members, links, and transmission elements for receiving the first input (yaw) and the second input (pitch) are shown to be analogous or similar. In general, different types of input members and/or transmission elements could be used for the first input versus the second input and they need not be similar or analogous.

Referring to FIG. 28E, another embodiment is illustrated which shows a single input member, which may be directly or indirectly coupled to the first swashplate 1124, and may be used to provide both the first input and the second input to the swashplate 1124. For example, a handle or input lever 1171 can be directly coupled to the first swashplate 1124 and may be used to provide any desired combination of the first input and the second input (i.e. yaw and pitch rotations) to the transmission system 1110.

In yet another embodiment shown in FIG. 28F, the articulation input transmission 1111 could comprise a set of pushrods. For example, a first pair of pushrods (PR21 and PR22) may be provided to rotate the first swashplate 1124 in pitch rotation. The first pair of pushrods PR21, PR22 is driven by a second input member 1118. Although not shown, a second pair of pushrods may be provided and driven by a first input member to rotate the first swashplate 1124 in yaw rotation.

Furthermore, while the first, second, and third input members in FIG. 21 are shown to receive rotational input motions, these input members could be easily configured to receive linear input motions such as shown in the case of Input 1 and Input 2 in FIG. 28B and FIG. 28C.

Similarly, Input 3 could be provided by a linear input motion and this input motion could be transmitted to the roll rotation of the shaft (about axis 1303) using a rack and pinion gear arrangement.

The rotational input motions could be provided via levers, motors, pulley, gears, etc. The linear input motions could be provided by fluid piston-cylinders, linear motors, voice coil actuators, piezoelectric actuators, or motors with leadscrew or ballscrew, etc. Other actuator types can include electric, electromagnetic, air motor/turbine, fluidic motor/turbine, electrostatic. In any of these cases or the embodiments shown in the figures, there may be additional transmission elements between the actuator and the input members. These transmission elements could include cables, pulleys, spools, gears (spur, bevel, helical, planetary, etc.), leadscrew/ballscrew, belts, linkages, chains, etc.

In general, the input members and transmission elements for the first input, the second input, and the third input could be similar (analogous) or be different.

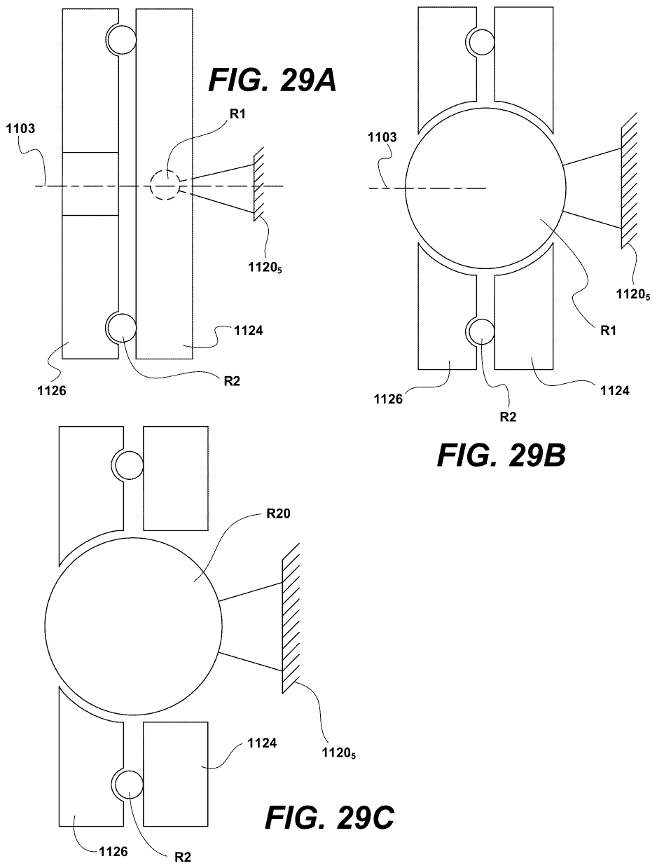

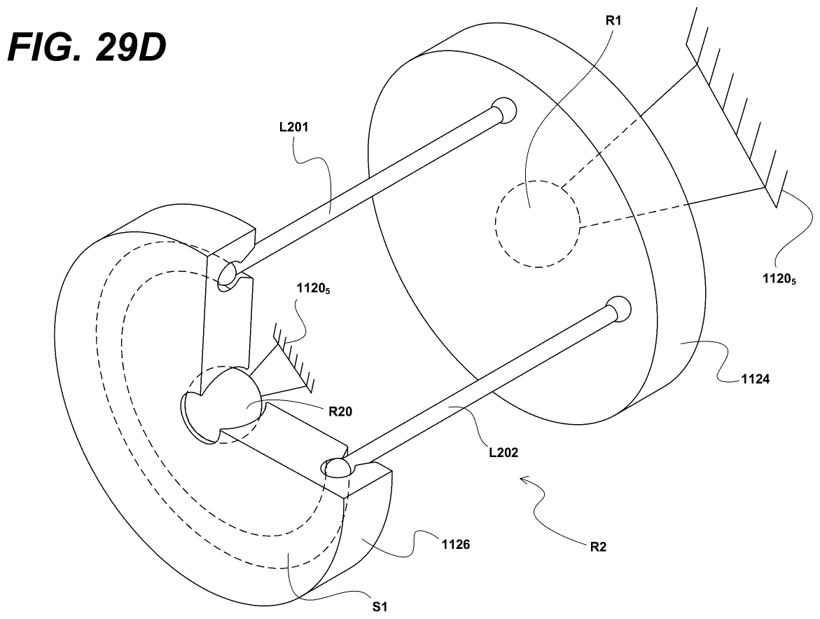

Furthermore, there can be different ways of coupling the first and second swashplates 1124, 1126 to each other and to ground 1120₅. Referring to FIG. 29A, an embodiment shows the arrangement used in FIG. 21A, where the first swashplate 1124 is coupled to ground 1120₅ via the first rotational joint R1, which may be a 2 DoF rotation joint (such as a gimbal, universal joint, cardan joint, CV joint etc.). The second swashplate 1126 is coupled to the first swashplate 1124 via the second rotational joint R2 that allows one rotational DoF (roll rotation) about axis 1103 and constraints articulation (i.e. yaw and pitch rotations). The second rotational joint R2 can comprise one or more rolling element bearings (e.g. ball bearing, roller bearing), or one or more bushings, to support axial, radial, and/or moment loads.

Alternatively, as shown in FIG. 29B, both the first and second swashplates 1124, 1126 can be coupled to ground 1120₅ via the first rotational joint R1 which may be a 3 DoF joint (e.g. a spherical or ball and socket joint). The first swashplate 1124 is coupled to the second swashplate 1126 via the second rotational joint R2 that allows one rotational DoF (roll rotation) about axis 1103 and constraints articulation (i.e. yaw and pitch rotations).

In yet another alternative embodiment shown in FIG. 29C, the second swashplate 1126 may be coupled to Ground 11205 via a 3-DoF rotational joint R20. And, the first swashplate 1124 is coupled to the second swashplate 1126 via joint R2 that allows one rotational DoF (Roll rotation) about axis 103 and constraints articulation (i.e. Yaw and Pitch rotations).

In yet another embodiment shown in FIG. 29D, the first swashplate 1124 is coupled to the ground 1120₅ via the first rotational joint R1, which may be a 2-DoF joint (e.g. a universal joint, or cardan joint, etc.) and the second swashplate 1126 is coupled to ground 1120₅ via a 3-DoF rotational joint R20 (e.g. a spherical joint, a ball and socket joint, etc.). The first swashplate 1124 and second swashplate 1126 are coupled via links. Typically, at least three or more links are needed to transmit the pitch and yaw rotations from the first swashplate to the second swashplate, but only two are shown in this figure (L201 and L202). One end of each link L201, L202 may be coupled to the first swashplate 1124 via a two-DoF rotational joint (e.g. a universal joint, a cardan joint, a constant velocity or CV joint, etc.) or a three-DoF rotational joint (e.g. spherical joint, ball and socket joint, etc.). The other end of the links L201, L202 may be coupled to the second swashplate 1126 via a ball end that can slide in a circular slot S1. This arrangement ensures that the yaw and/or pitch rotations of the first swashplate 1124 are transmitted to the second swashplate 1126, while the second swashplate 1126 remains free to roll about axis 1203 with respect to the first swashplate 1124.

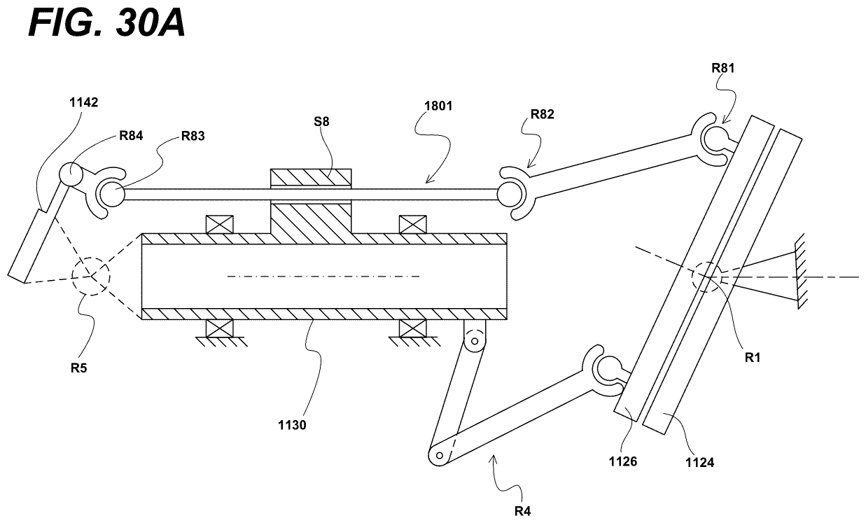

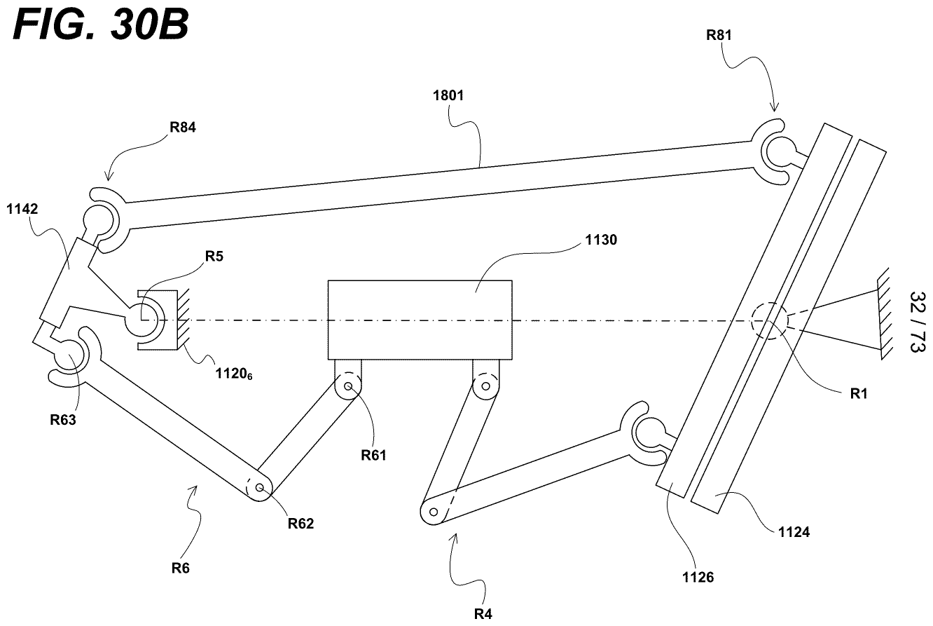

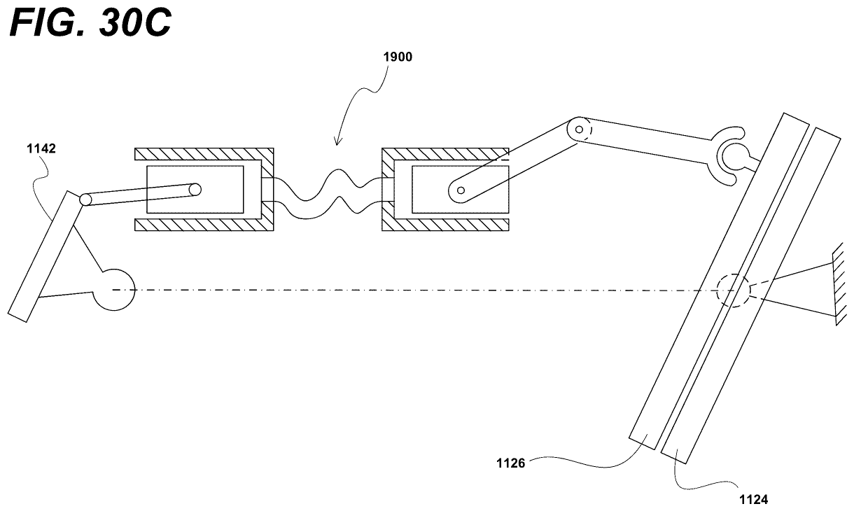

The output subsystem 1114 comprises an “articulation output transmission” that transmits the articulation of the second swashplate 1126 to articulation of the end effector (or distal manipulator) 1142. Referring to FIGS. 21A and 21B, the embodiment shows an “articulation output transmission” comprising cables. In an alternate embodiment as illustrated in FIG. 30A, the articulation output transmission may comprise a linkage. This linkage may comprise at least two pushrods, of which a first pushrod 1801 is shown. This push rod is coupled to the second swashplate 1126 via two joints, R81 and R82, each of which could be a 2 DoF rotational joint (e.g. universal or cardan joint) or a 3 DoF rotational joint (e.g. spherical or ball joint). The pushrod 1801 is coupled to the shaft 1130 via a slider interface 56. The other end of the pushrod 1801 may be coupled to the distal manipulator 1142 via another link and two joints R83 and R84, each of which could be a 2 DoF rotational joint (e.g. universal or cardan joint) or a 3 DoF rotational joint (e.g. spherical or ball joint). While one pushrod is shown (for transmitting pitch articulation), there can be second pushrod (not shown) in an orthogonal plane that transmits yaw articulation.

In yet another variation of this embodiment shown in FIG. 30B, pushrod 1811 is directly coupled between the second swashplate 1126 and distal manipulator 1142 via rotational joints R81 and R84, each of which could be a 2 DoF rotational joint (e.g. universal or cardan joint) or a 3 DoF rotational joint (e.g. spherical or ball joint). While a first pushrod 1811 is shown (for transmitting pitch articulation), there can be a second pushrod in an orthogonal plane that transmits yaw articulation (not shown). The distal manipulator 1142 in this embodiment is coupled to ground 1120₆ via the output rotational joint R5, which may be a 3-DoF rotational joint such as a spherical or ball joint. Also, the distal manipulator 1142 is coupled to the roll input 1128 or shaft 1130 via a second roll drive coupling R6 that is analogous to the first roll driving coupling R4. The second roll drive coupling R6 could also be an R-R-S chain comprising joints R61 (revolute), R62 (revolute), and R63 (spherical).

In yet another embodiment as illustrated in FIG. 30C, the articulation output transmission within the output system 1114, the articulation of the second swashplate 1126 could be transmitted to the articulation of the distal manipulator 1142 via a fluidic transmission system 1900.

Referring now to FIGS. 31A and 31B, another embodiment may include the output rotational joint R5 that couples the shaft 1130 to distal manipulator 1142. The output rotational joint R5 may be a bellows joint such that it allows relative articulation, as shown in FIG. 31B, but is stiff in torsion and therefore transmits roll rotation about the articulated axis 1403. Any bearing mentioned here could be a rolling element bearing, or a bushing, or a flexure bearing, air bearing, fluid bearings, or magnetic bearing.

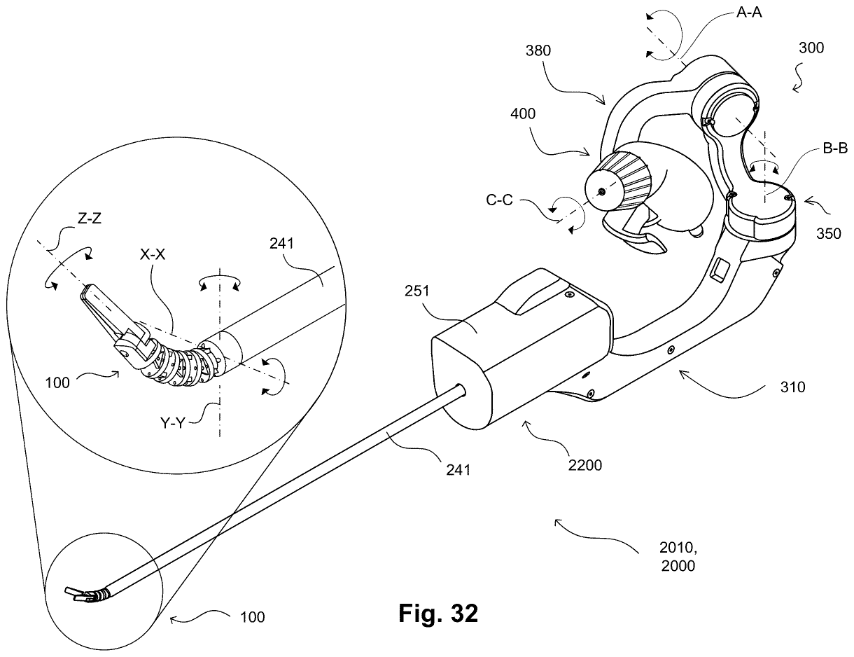

Referring now to FIG. 32, the Device 2010 features a proximal end with a Handle Assembly 400 controlled by a user, and a distal end with a Distal Manipulator 100 with at least three controllable rotational degrees of freedom (DoF). These rotational degrees of freedom are defined in FIG. 32 as rotation about Yaw Input Axis B-B, rotation about Pitch Input Axis A-A, and rotation about Roll Input Axis C-C. The Distal Manipulator's Pitch Output Axis X-X and Yaw Output Axis Y-Y are defined with respect to the Chassis Subassembly 310. The Distal Manipulator's 100 Roll Output Axis Z-Z is aligned with and fixed to the Distal Manipulator 100. In other words, the Distal Manipulator 100 revolves about its articulated Roll Output Axis Z-Z. The Roll Output Axis Z-Z is defined by rotations about the Pitch Input Axis A-A and the Yaw Input Axis B-B. The control of the rotations about the Yaw Input Axis B-B, Pitch Input Axis A-A, and Roll Input Axis C-C is decoupled.

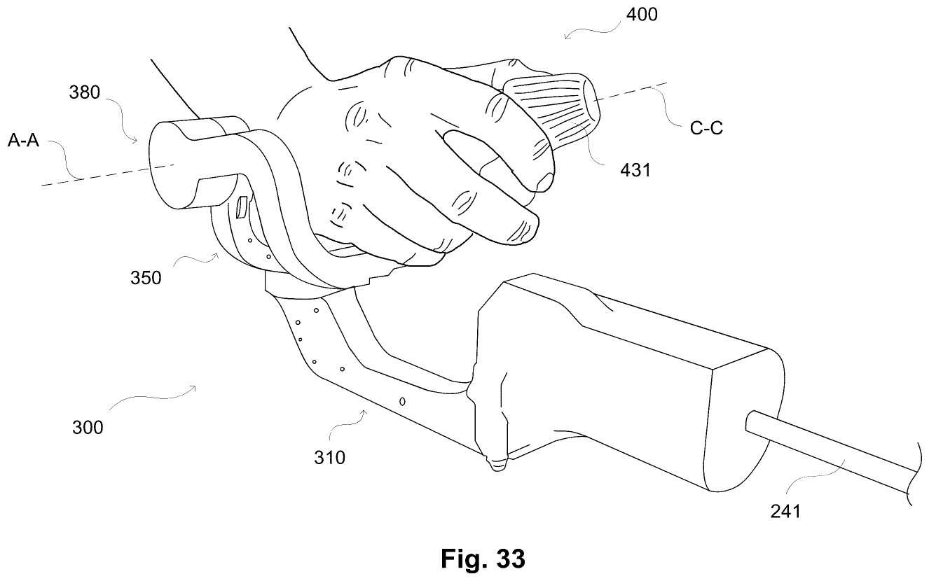

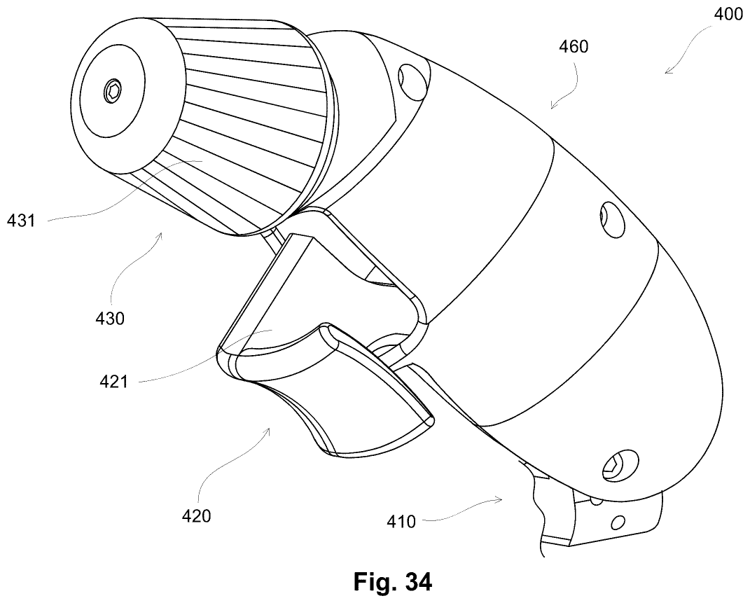

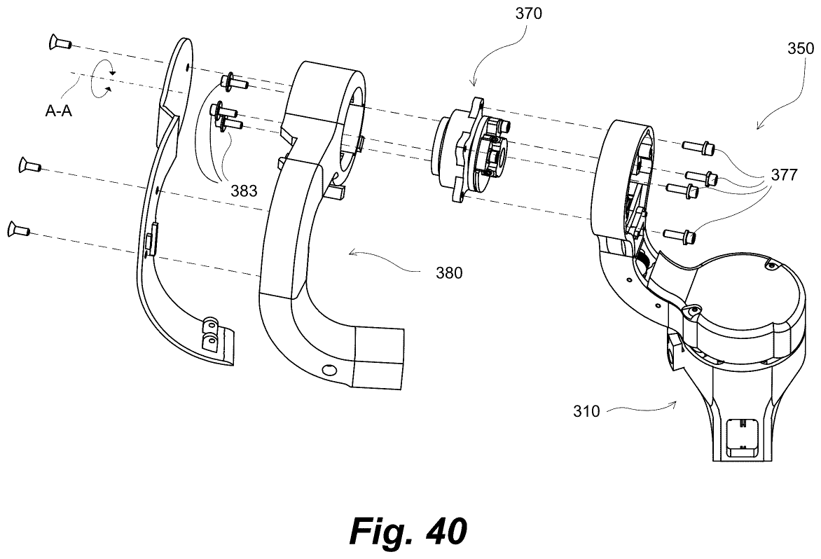

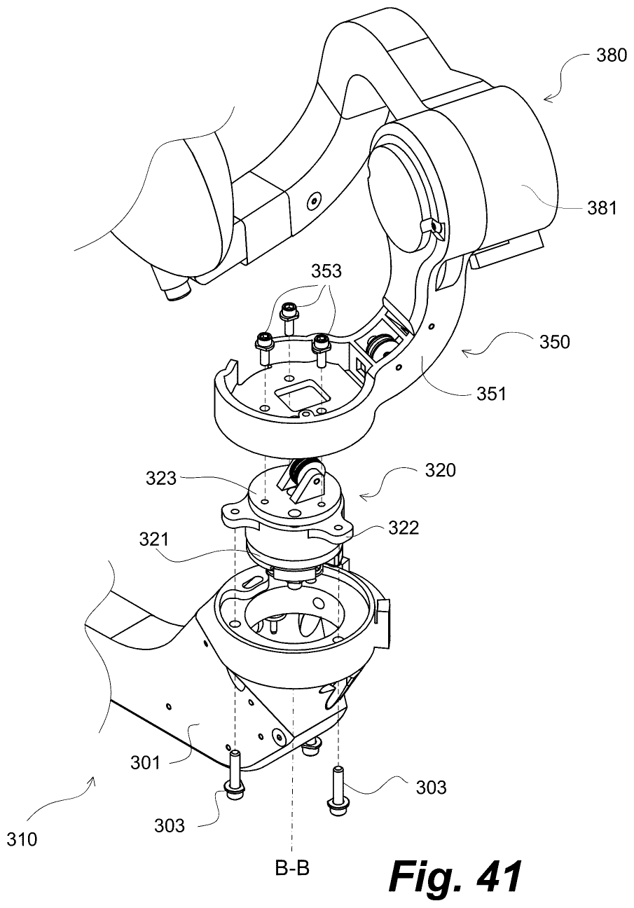

As illustrated in FIGS. 32 and 33, the control of the DoFs about the Pitch Input Axis A-A and Yaw Input Axis B-B is performed by a user's hand manipulating the Handle Assembly 400, which is coupled to and controls Input Linkage Assembly 300, which may feature two links (for example a Pitch Link Subassembly 380 and a Yaw Link Subassembly 350) and two revolute joints (e.g. a Pitch Joint Subassembly 370 as seen in FIG. 40 and a Yaw Joint Subassembly 320 as seen in FIG. 41). The rotational axes of the revolute joints may be orthogonal to each other and may correspond to the Yaw Input Axis B-B and Pitch Input Axis A-A. When the Handle Assembly 400 is held or grasped by a user, this arrangement allows the user to manipulate the linkage by rotating their wrist in flexion and extension (corresponding to rotation about the Yaw Input Axis B-B), and radial deviation and ulnar deviation (corresponding to rotation about the Pitch Input Axis A-A). The links are shaped to comfortably accommodate the hand and wrist of the user while holding the Handle, and allow for a natural interface between the user and the Device 2010 via the Handle Assembly 400. In some instances, the Yaw Input Axis B-B and/or the Pitch Input Axis A-A may pass close to the user's wrist, as the user holds the Handle Assembly. The rotation of the Distal Manipulator about Roll Output Axis Z-Z is controlled by rotating the Dial 431 on the Handle 400 with the thumb and index finger about Roll Input Axis C-C.

The Handle Assembly 400 is the portion of the Device 2010 that the user interfaces with in order to control the three rotational DoF of the Distal Manipulator 100. By manipulating the Handle Assembly 400, the user is able to comfortably and fluidly control the two articulation DoFs (rotations about the Yaw Input Axis B-B and Pitch Input Axis A-A) and one Roll DoF at the Distal Manipulator simultaneously in order to perform complex motions and maneuver. The Handle Assembly 400 comprises multiple mechanisms and subassemblies to ensure that the user can optimally control the three rotational DoF of the Device. This means that these mechanisms and subassemblies allow the user to impart (or input) precise motions at the Handle Assembly 400 and produce precise output motions at the Distal Manipulator assembly 100, and that the mechanisms and subassemblies minimize the amount of effort that the user must exert in order to do so. In the preferred embodiment, this is achieved by the design of the Handle Assembly 400, which comprises the Handle Body Subassembly 460, Pitch Link Interface 410, Trigger Subassembly 420, and Dial Subassembly 430, as shown in FIG. 34.

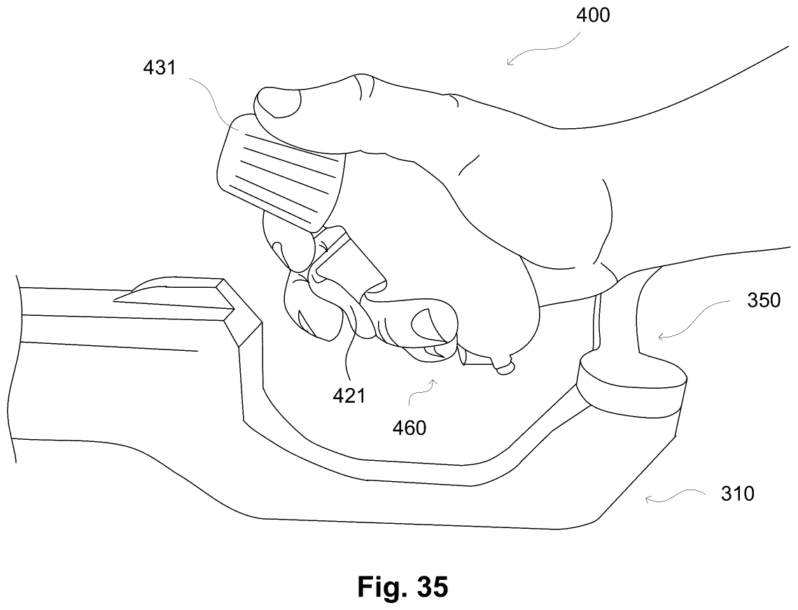

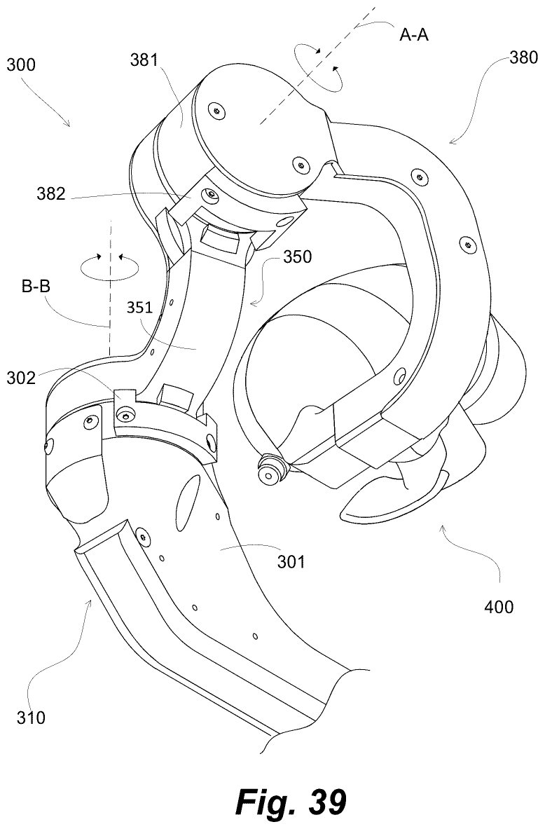

In the preferred embodiment, shown in FIG. 35, the user may control the rotation of the Handle about the Yaw Input Axis B-B and/or the Pitch Input Axis A-A by grasping the Handle Assembly 400 via the Handle Body 460, with the little finger and ring finger wrapping towards the underside of the Handle Body 460. The user may then rest their middle finger on the Trigger 421, and they may rest their index finger and thumb on the Dial 431. The user's grasp on the Handle Body 460 allows the user to articulate the Handle Assembly 400 along the two articulation DoFs allowed by the Input Linkage Assembly 300 (FIG. 39). For example, the user can articulate their hand left and right about their wrist (flexion/extension), with respect to their forearm. This articulation of the user's hand is transmitted to the Handle Body 460 since the user is grasping the Handle Body 460, causing the Handle Body 460 to articulate in Yaw rotation with respect to the Chassis Subassembly 310. The user's articulation inputs (i.e. Yaw, Pitch, or any combination thereof) are transmitted from the Handle Body 460 to the Input Linkage Assembly 300 via the Pitch Link Interface 410, which is a structural interface between the Handle Body 460 and the Pitch Link 381. Such an interface may be created via alignment pins, screws, press fits, friction joints, adhesives, etc.

The user's index finger and thumb may actuate (e.g. turn) the Dial 431, which produces an input for the Roll DoF, which is subsequently transmitted and translated into an rotation about the Roll Output Axis Z-Z by Distal Manipulator 100 via the Dial Subassembly 430, Mechatronics System 700 (e.g., that described with respect to FIG. 20B), Roll Transmission Subassembly 204, and Tool Shaft Subassembly 203. One or more of the user's fingers and/or thumb may actuate/engage the Trigger 421. Actuation of the Dial 431 and Trigger 421 can be done by the user in any articulated position of their hand when using the Device. The controls of the Handle Assembly 400 are designed to allow the user to comfortably provide articulation and Roll inputs simultaneously to the Device.

The user's fingers and hand do not need to overextend or apply significant pressure in order to use the DoF input controls or maintain their grasp on the Handle Assembly 400, and the user does not have to re-adjust their grip in order to properly provide input to the multiple DoFs simultaneously. In the preferred embodiment, this is achieved by a Handle Assembly 400 shape such that different fingers or groups of fingers perform separate and distinct control functions to control the Device DoFs. For example, the user's ring finger, little finger, and palm grasp the Handle Body Subassembly 460 as seen in FIG. 35, such that the user may control the articulation DoFs (rotations about the Yaw Input Axis B-B and Pitch Input Axis A-A) of the Device. The user's middle finger may rest on the Trigger 421 as seen in FIG. 35, and actuates the Trigger 421 against the user's palm which rests on the Handle Body Subassembly 460. The user's index finger and thumb grasp and rotate the Dial 431 as seen in FIG. 35 in order to provide Roll input into the Device. The Dial 431 is both positioned and shaped to allow the user's fingers and/or thumb to rest on the Dial 431 in their natural resting position (i.e. where they would rest if the user's hand were at ease). The Dial 431 is shaped so that the user may also adjust their fingers to use a wide range of grips on the Dial 431, depending on their preferred technique of inputting a rolling motion. The Dial's 431 shape and positioning also allows the user to turn the Dial 431 over a large angular range (in Roll rotation) without the user having to reposition or strain their fingers. This ergonomic design of the Dial 431 allows the user to provide precise, controlled, and fluid Roll rotation/Roll DoF input to the Device. In alternative embodiments, the design may allow for the user to use any combination of fingers to actuate/engage the various inputs to the Device. For example, in an alternative embodiment the middle finger can be used to provide Roll inputs to a Dial 431 located along the middle of the Handle Body Subassembly 460 (instead of its distal end).

The Dial 431, Trigger 421, Handle Body Subassembly 460, or their analogies in alternative embodiments may also be customizable, replaceable, or be designed to have customizable inserts that further allow the Handle Assembly 400 design to be adjusted to fit a given user's specific needs. The Handle Body Subassembly 460 may be built out of several separate components that are assembled together.

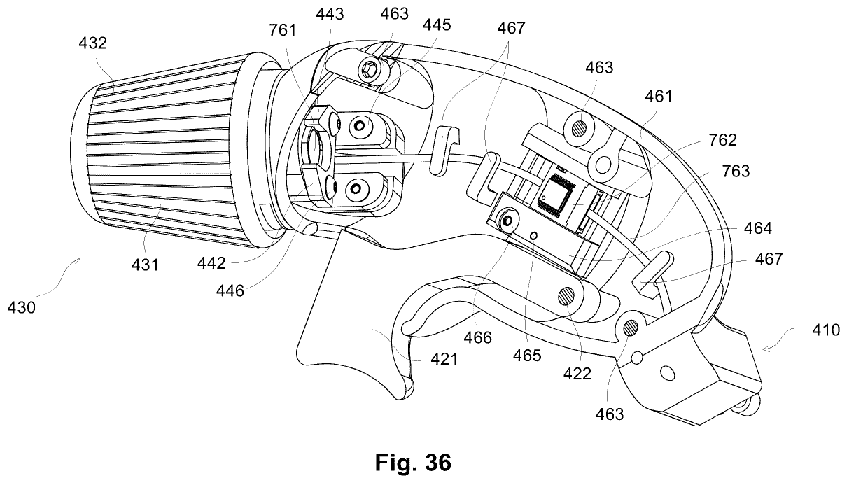

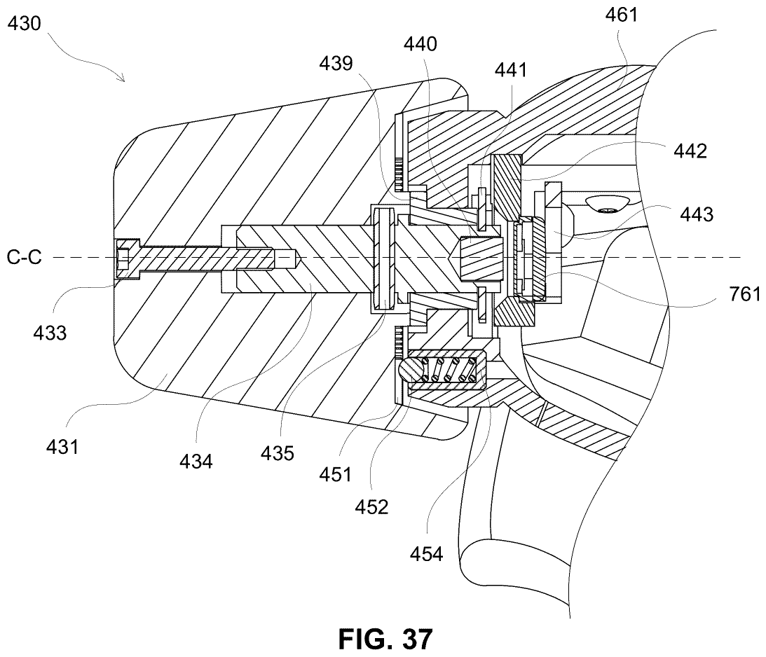

The Handle Assembly 400 captures the user's Roll input and transmits it to other mechanisms and components in the Device 2010 that then produce a Roll output at the Distal Manipulator 100. There are many embodiments of mechanisms that could capture and translate the Roll input within the Handle, and in the preferred embodiment this is achieved by the Dial Subassembly 430, which is shown in FIGS. 36 and 37. In this embodiment, the user may be grasping the Dial 431 with their fingers, and may apply a rotational input to the Dial 431 about the Roll Input Axis C-C, which is detected and measured by the Dial Encoder 761 (e.g., the dial encoder 526a/2126z described with respect to FIG. 20B) and transmitted to the Mechatronics System 700, which then produces a corresponding rotation about the Roll Output Axis Z-Z by the Distal Manipulator 100. In order to do this, the Dial 431 transmits rotational motion about Roll Input Axis C-C to the Dial Shaft 434, since the two components are rigidly connected via Dial Screw 433, and Dial Cross Pin 435. The Dial Shaft 434 is coupled to the Handle Body Subassembly 460 via the Dial Bushing 439. The Dial Shaft 434 is constrained in translation along the Roll Input Axis C-C to the Dial Bushing 439 via the Dial Shaft Snap Ring 441. The interface between the Dial Shaft 434 and the Dial Bushing 439 offers 1 DoF, which is a rotation DoF about the Roll Input Axis C-C.

A Dial Magnet 440 is mounted at the end of the Dial Shaft 434. As the Dial Shaft 434 rotates, the Dial Magnet 440 also rotates concentrically (about the Roll Input Axis C-C) with respect to the Dial Encoder 761, which is mounted to the Handle Body 460 via the Encoder Mount 442, Encoder Mount Screws 445, Encoder Clamp 443, and Encoder Clamp Screws 446. Dial Encoder 761 is electrically connected to the Encoder Line Driver 762, and the connecting electrical wires are constrained to the Handle Body Subassembly 460 by Wire Hooks 467. The Encoder Line Driver 762 interfaces with the Mechatronics System 700 via Wiring Harness 763, which is partially secured to the Handle Body Subassembly 460 by a Wire Hook 467 before passing through the Pitch Link Interface 410.

In the preferred embodiment, the Roll input that the user applies to the Dial 431 is captured by the interaction between the Dial Magnet 440 and the Dial Encoder 761. When the Dial Magnet 440 rotates, the orientation of the constant magnetic field that it emits changes with respect to the orientation of the Dial Encoder 440, which is capable of detecting this change in orientation at a high resolution. The Dial Encoder 761 transmits this information to the Mechatronics System 700, which then uses this information to generate an Roll rotation output at the Distal Manipulator 100, which results in a well defined relationship between the angular rotation of the Dial 431 in the Roll direction (relative to Handle Body Subassembly 460) and the angular rotation of the Distal Manipulator 100 in the Roll direction (relative to the Chassis Subassembly 310). This relationship could be a constant ratio of any magnitude, such as 1:1, or it could be more complex e.g. a different transmission ratio of 1:2, 1:3, 1:4, 2:1, 3:1, 4:1 etc. or any other desired transmission ratio.

In alternative embodiments, the sensing of the Dial position could be performed by any sensor that can detect changes in position, such as mechanical encoders, optical encoders, linear encoders, resolvers, potentiometers, or other sensors. In the preferred embodiment, the output of the Dial Subassembly 430 is an electrical signal that is sent to and processed by the Mechatronics System 700, but there are many alternative embodiments for methods of capturing the user's Roll input and translating that into a Roll output at the Distal Manipulator 100. For example, the rotation of the Dial 431 and the Distal Manipulator 100 could be coupled mechanically, with mechanisms such as torsion cables, hydraulically or pneumatically, with mechanisms such as fluid couplings, magnetical couplings, linkage mechanisms, gear assemblies, or electronically, with other types of mechatronic sensors and actuators.

In the preferred embodiment, the design of the Device has the ability to perform an infinite amount of rotation of the Distal Manipulator 100 about Roll Output Axis Z-Z in either direction of the rotation (clockwise or counter clockwise). This allows the user to adjust the rotation of the Distal Manipulator 100 about the Roll Output Axis Z-Z by as much as needed in any situation, which allows them to perform complex, extended movements without requiring them to reset their Roll input to some nominal or starting or initial position. The ability for the user to apply as much Roll input as needed enables their precise control of the Device, because it ensures that no articulation of the Distal Manipulator 100 will be inhibited by their inability to provide additional roll rotation to the Distal Manipulator 100, and vice versa.

The preferred embodiment of the Dial Subassembly 430 accommodates this by having no mechanical limit on how many rotations the Dial 431, or any components that are attached to it, can perform. The Dial Magnet 440 can rotate by an infinite amount of displacement relative to the Dial Encoder 761 (about Roll Input Axis C-C) in either direction, and so accordingly the Dial Subassembly 430 can continuously and indefinitely relay signals representing changes in angular position to the Mechatronic System 700.

In the preferred embodiment, the Dial Subassembly 430 provides the user with haptic feedback for their Roll input. With this feature (described next), the user is able to accurately assess or gauge the amount by which they are changing the angular position of the Tool Shaft Subassembly 203 and Distal Manipulator 100 without looking at the Dial 431, due to tactile features in the Dial Subassembly 430 that physically convey to the user how much they have changed the angular position of the Dial 431.

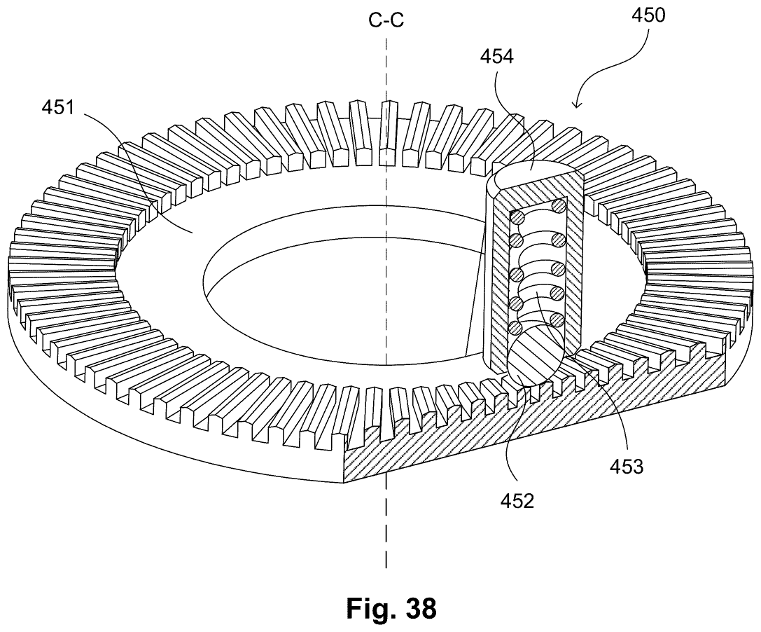

In the preferred embodiment of the Dial Subassembly 430, this is achieved in two ways: with the Detent Mechanism 450 (see FIG. 38) and the Dial Knurling 432 (see FIG. 36). The Dial Knurling 432 is intended to maximize the user's grip in the Dial 431, and minimize the risk of the Dial 431 slipping out of their grasp unexpectedly, which could lead to an undesired Roll input. The Dial Knurling 432 is also designed such that the user may discern each of the individual ribs or rib-like features that compose it. The user may be able to count, whether consciously or subconsciously, the amount of ribs of the Dial Knurling 432 that they feel rolling underneath their fingers as they rotate the dial, which gives them a periodic tactile sensation in fine increments which correlates to the amount they have rotated the Dial 431. The Detent Mechanism 450 provides the user with haptic feedback by producing a mechanical impulse into the Dial 431 at a fine, periodic interval, which the user can feel in their fingers. In the preferred embodiment, the Detent Mechanism 450, shown in FIG. 38 below, comprises a Detent Plunger 452, a Detent Spring 453, a Detent Body 454 which retains the former two components, and a Detent Track 451, which is rigidly attached to the Dial 431. As the user turns the Dial 431, which rotates about Roll Input Axis C-C, the Detent Track 451 also rotates about this axis and passes by the Detent Body 454 since the Detent Body 454 is attached to the Handle Body Subassembly 460. As this happens, the Detent Spring 453 pushes the Detent Plunger 452 into each of the valleys between the teeth of the Detent Track 451 as they pass by, and the peaks of the teeth push the Detent Plunger 452 back into the Detent Body 454. The only stable equilibrium in this cycle is when the Detent Plunger is resting in a valley between teeth, and so the Dial 431 will tend to come to rest in this condition. This creates a fixed number of rest conditions throughout one full rotation of the Dial 431, and every time the Detent Mechanism 450 is moved from one position to the next, it produces an impulse into the Dial 431 as the Detent Plunger 452 attempts to achieve stable equilibrium. This impulse is periodic and may be felt by the user, thus providing them with a tactile indication of changes in Dial 431 angular rotation (i.e., in Roll).

Alternative embodiments may provide haptic feedback to the user for the Roll input mechanically by alternative methods for producing impulses or sense of movement into the users fingers, such as by connecting the Roll input to the Roll output via a load path that allows the mechanical vibrations experienced by the output to reverberate back to the Roll input, where the user can detect them with their fingers. A non-visual relationship between the movement of the Roll input and the roll output could also be established via sound; if the Roll input generated a sound that changed in volume/pitch depending on the magnitude by which or how fast the Roll input was changed by the user, the user would be able to gauge how they are controlling the Roll output without having to visually inspect the Roll input.

FIG. 32 shows the Input Linkage Assembly 300 in the context of the full device, and FIG. 39 depicts the Input Linkage Assembly 300 as viewed in an isometric view from the proximal end. The Input Linkage Assembly 300 is responsible for transmitting the user's articulation inputs (e.g. Yaw, Pitch, or any combination thereof) from the Handle Assembly 400 to the Swashbox Assembly 2200 (see FIG. 50). The inputs that are transmitted from the Handle assembly 400 to the Swashbox Assembly via the Input Linkage Assembly 300 are the rotation of the Handle Assembly 400 with respect to the Chassis Subassembly 310 about the Yaw Input Axis B-B and the rotation of the Handle Assembly 400 with respect to the Yaw Link Subassembly 350 about the Pitch Input Axis A-A.

The Input Linkage Assembly 300 receives the articulation (any combination of rotation about the Yaw Input Axis B-B or rotation about the Pitch Input Axis A-A from the user) of the Handle Assembly 400 into two rotations, namely the rotation of the Handle Assembly with respect to the Chassis Subassembly 310 about the Yaw Input Axis B-B and the rotation of the Handle Assembly 400 with respect to the Yaw Link Subassembly 350 about the Pitch Input Axis A-A. The user can articulate their Hand (and the Handle Assembly 400) about their wrist in any combination of flexion/extension, and radial/ulnar deviation. The Input Linkage Assembly 300 resolves this articulation of the user's Hand (and therefore the Handle Assembly 400) into two rotations, one about the Yaw Input Axis B-B and the Pitch Input Axis A-A.

As seen in FIG. 39, the Input Linkage Assembly 300 comprises the Yaw Link Subassembly 350, the Pitch Link Subassembly 380, and the proximal portion of the Chassis Subassembly 310. The Pitch Link Subassembly 380 is coupled to the Handle Assembly 400 via the Pitch Link Interface 410 (See FIG. 36). In some instances, this coupling could be a rigid structural attachment or connection. Therefore, any rotations of the Handle Assembly 400 about the Pitch Input Axis A-A with respect to the Yaw Link Subassembly 350 or the Yaw Input Axis B-B with respect to the Chassis Subassembly 310 result in corresponding rotations of the Pitch Link Subassembly 380 about the Pitch Input Axis A-A and Yaw Input Axis B-B with respect to the aforementioned respective subassemblies.

FIG. 40 shows an exploded view of the subassemblies related to the rotation about the Pitch Input Axis A-A received from the Handle Assembly 400. The Pitch Joint Subassembly 370 enables the relative motion between the Pitch Link Subassembly 380 and the Yaw Link Subassembly 350 such that the only rotation of the Pitch Link Subassembly 380 with respect to the Yaw Link Subassembly 350 is about the Pitch Input Axis A-A is allowed. Therefore, the rotation of the Handle Assembly 400 (see FIG. 39) and Pitch Link Subassembly 380 with respect to the Chassis Subassembly 310 about the Yaw Input Axis B-B result in equivalent rotation of the Yaw Link Subassembly 350 with respect to the Chassis Subassembly 310 about this same axis. The Yaw Input Axis B-B will always be fixed in the Yaw Link Subassembly 350 and rotates about Yaw Input Axis B-B when the Handle Assembly 400 is rotated in about the Yaw Input Axis B-B.

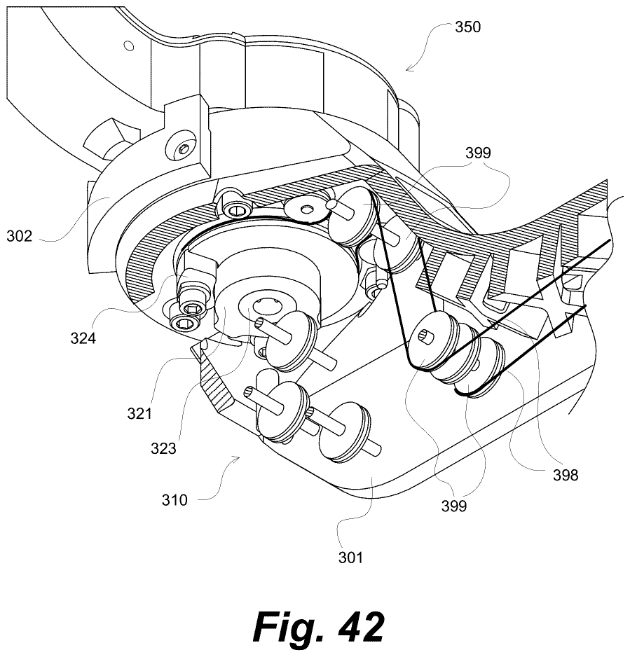

FIG. 41 presents the subassemblies that make up the Yaw Transmission Subassembly in an exploded view. The Yaw Transmission Subassembly transmits the relative rotation of the Handle Assembly 400 about the Yaw Input Axis B-B with respect to the Chassis Subassembly 310 to the Swashbox Assembly 2200 (not shown). The Yaw Transmission Subassembly is composed of the Yaw Link Subassembly 350, the Yaw Joint Subassembly 320, and the Yaw Input Cable 398 (shown in FIG. 42). FIG. 42 depicts a section view of the proximal end of Chassis Subassembly 310 viewed from below further illustrating the Yaw Transmission Subassembly.

As seen in FIG. 41, the Yaw Joint Subassembly 320 mates the Yaw Link Subassembly 350 to the Chassis Subassembly 310 such that a degree of freedom (rotation about the Yaw Input Axis B-B) is allowed. The Yaw Joint Subassembly 320 also couples the rotation of the Yaw Link Subassembly 350 with respect to the Chassis Subassembly 310 about the Yaw Input Axis B-B to the Yaw Input Cable 398.

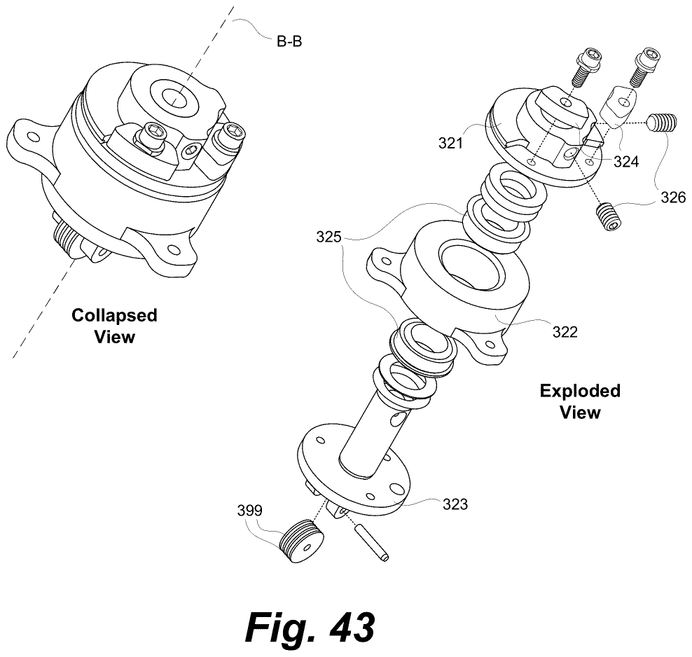

FIG. 43 shows the Yaw Joint Subassembly 320 isolated by itself, in a collapsed (i.e. assembled) view and an exploded view.

Referring to FIG. 41 and FIG. 43, the Yaw Joint Shaft 323 is secured to the Yaw Link 351 via Yaw Link Mounting Screws 353. The Yaw Joint Housing 322 is secured to the Chassis 301 via Yaw Joint Mounting Screws 303. Two Ball Bearings 325 allow relative rotation between Yaw Joint Shaft 323 and Yaw Joint Housing 322 about the Yaw Input Axis B-B. The Yaw Link Subassembly 350 and subsequently the Pitch Link Subassembly 380, Pitch Joint Subassembly 370 and Handle Assembly 400 are therefore allowed to rotate with respect to the Chassis Subassembly 310 about the Yaw Input Axis B-B.

As shown in FIG. 42, the Yaw Input Cable 398 is secured to the Yaw Joint Shaft 323 via clamping the ends of the Yaw Input Cable 398 to the Yaw Driving Pulley 321 with two Yaw Cable Clamps 324. The Yaw Driving Pulley 321 is attached to the Yaw Joint Shaft 323 via two Screws 326 (see FIG. 43).

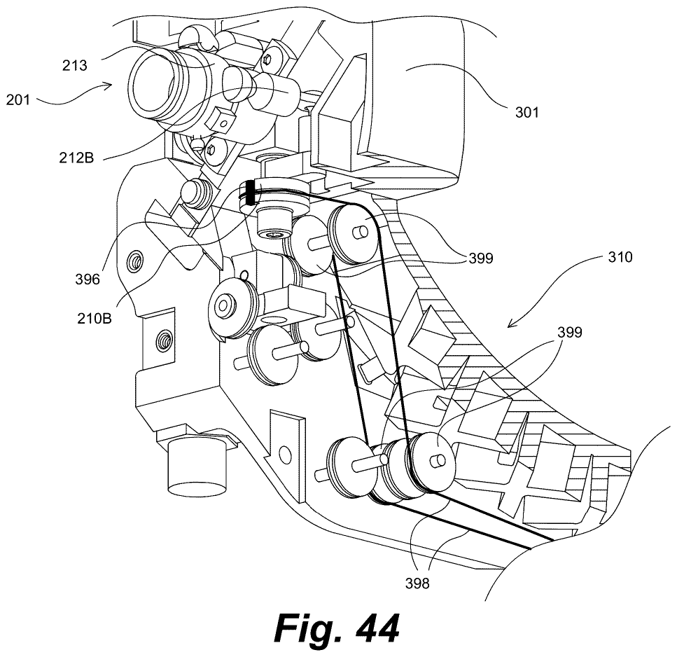

The mounting of the Yaw Link 351 to the Yaw Joint Shaft 323 to the Yaw Driving Pulley 321 to the Yaw Input Cable 398 defines the path from which the Yaw rotation of the Handle Assembly 400 and Pitch Link Subassembly 380 is transmitted to the Yaw Input Cable 398. The rotation of the Yaw Driving Pulley 321, and therefore the Yaw Link 351 and Yaw Joint Shaft 323, about the Yaw Input Axis B-B with respect to the Chassis Subassembly 310 is transmitted through the Yaw Input Cable 398 to the Yaw Driven Pulley 210B in the Swashbox Assembly 2200 via positive engagement between a Medial Crimp 396 on the Yaw Input Cable 398 and a groove in the Yaw Driven Pulley 210B. This interface between the Yaw Input Cable 398 and Yaw Driven Pulley 210B about the Yaw Input Axis B-B is depicted in FIG. 44 which provides a section view of the Chassis Subassembly 310 with the Articulation Subassembly 201 shown looking from the distal end towards the proximal end.

Referring to FIG. 44, the Yaw Driven Pulley 210B is located in the Swashbox Assembly 2200 at the distal end of the Chassis Subassembly 310. The rotation of the Yaw Driven Pulley 210B acts as the input to the Swashbox Assembly (2200) corresponding to the Handle Assembly's 400 rotation about the Yaw Input Axis B-B with respect to the Chassis Subassembly 310 as previously shown in FIG. 32.

Referring to FIG. 42 and FIG. 44, to suitably route the Yaw Input Cable 398 along the chassis 301, multiple Redirect Pulleys 399 (also known as Idler Pulleys) may be used within the Chassis Subassembly 310. The materials of these Idler Pulleys 399 and Idler Shafts are selected such that the friction between the Idler Pulleys 399 and the Idler Shafts is minimized.

Referring to FIG. 39, in some instances it may be desirable to limit the range of rotation of the Yaw Link Subassembly 350 with respect to the Chassis Subassembly 310 about the Yaw Input Axis B-B as to avoid stressing elements/components of the Yaw Transmission Subassembly. For example, failure to limit the aforementioned range of rotation in this situation can result in unnecessary stress on the Yaw Input Cable 398 and the rest of the Yaw Transmission Subassembly. To mitigate this risk, a Yaw Hardstop 302 is mounted to the Chassis 301. The Yaw Hardstop 302 makes positive contact with a boss on the Yaw Link 351 when the range of rotation is met in either direction. This is simply one location for such a range limiting feature. Yaw Hardstops 302 can be located anywhere else along the Yaw Transmission Subassembly.

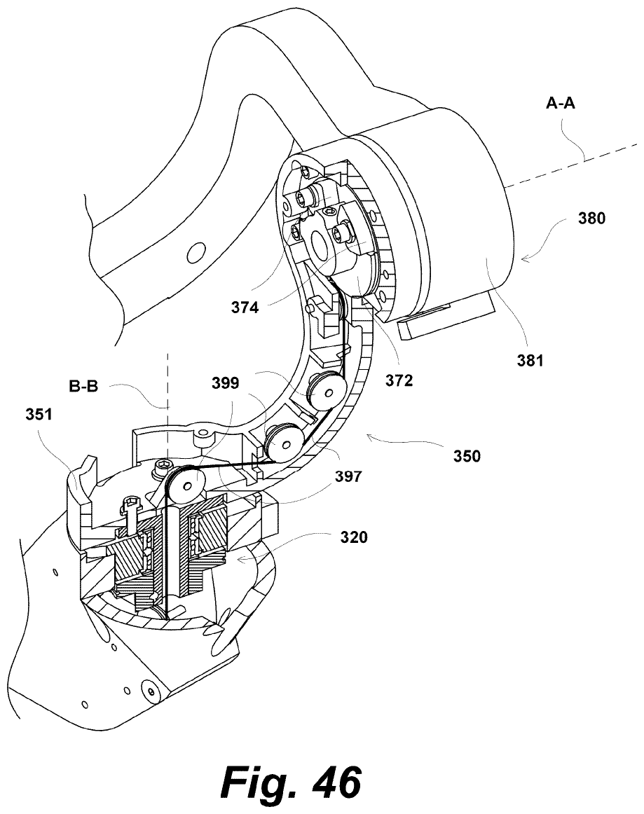

Referring back to FIG. 32 and FIG. 40, the relative rotation of the Handle Assembly 400 with respect to the Chassis Subassembly 310 about the Pitch Input Axis A-A is transmitted to the Swashbox Assembly 2200 via the Pitch Transmission Subassembly. An exploded view of the subassemblies that make up the Pitch Transmission are shown in FIG. 40. The Pitch Transmission Subassembly includes the Pitch Link Subassembly 380, the Pitch Joint Subassembly 370, and the Pitch Input Cable 397 (shown in FIG. 46). The Pitch Joint Subassembly 370 provides a mate between the Pitch Link Subassembly 380 and the Yaw Link Subassembly 350 such that a rotational degree of freedom (Pitch) between the two is allowed about the Pitch Input Axis A-A. The Pitch Joint Subassembly 370 also couples the rotation of the Pitch Link Subassembly 380 with respect to the Yaw Link Subassembly 350 about the Pitch Input Axis A-A to the Pitch Input Cable 397.

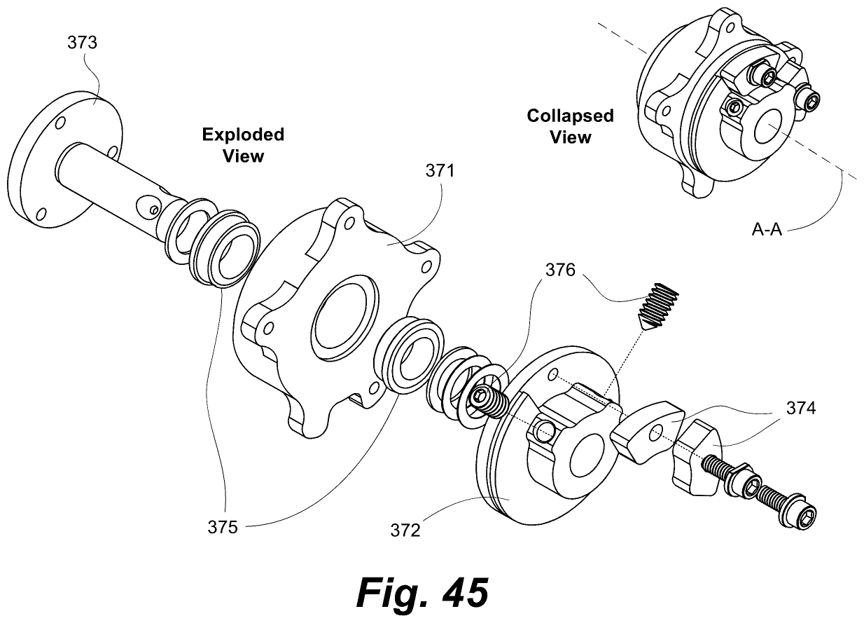

Referring to FIG. 40 alongside an exploded view of the Pitch Joint Subassembly 370 in FIG. 45, the Pitch Link 381 is rigidly secured to the Pitch Joint Shaft 373 via Screws 383. The Pitch Joint Housing 371 is secured to the Yaw Link Subassembly 350 via Screws 377. Two Ball Bearings 375 allow relative rotation between Pitch Joint Shaft 373 and Pitch Joint Housing 371 about the Pitch Input Axis A-A. The Pitch Link Subassembly 380 is therefore allowed to rotate with respect to the Yaw Link Subassembly 350 about the Pitch Input Axis A-A.

FIG. 46 depicts a section view of the Yaw Link Subassembly 350.

Referring to FIG. 45 and FIG. 46, the Pitch Driving Pulley 372 is secured to the Pitch Joint Shaft 373 via Screws 376. The Pitch Input Cable 397 is secured to the Pitch Joint Shaft 373 via clamping the ends of the Pitch Input Cable 397 to the Pitch Driving Pulley 372 with two Pitch Cable Clamps 374. The mounting of the Pitch Link Subassembly 380 to the Pitch Joint Shaft 373 to the Pitch Driving Pulley 372 to the Pitch Input Cable 397 defines the path from which rotation of the Handle Assembly 400 and Pitch Link Subassembly 380 about Pitch Input Axis A-A is transmitted to the Pitch Input Cable 397 within the Pitch Transmission Subassembly.

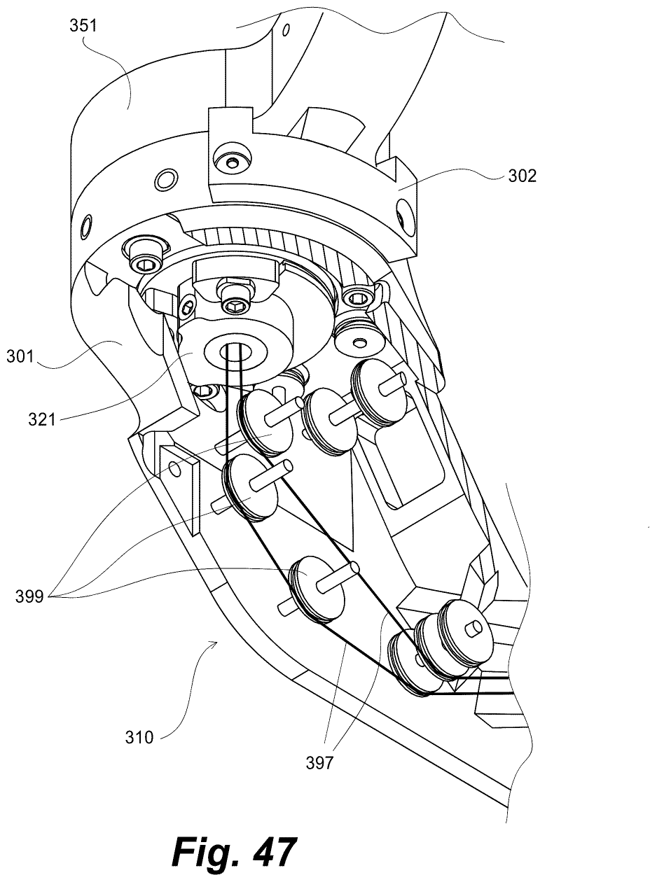

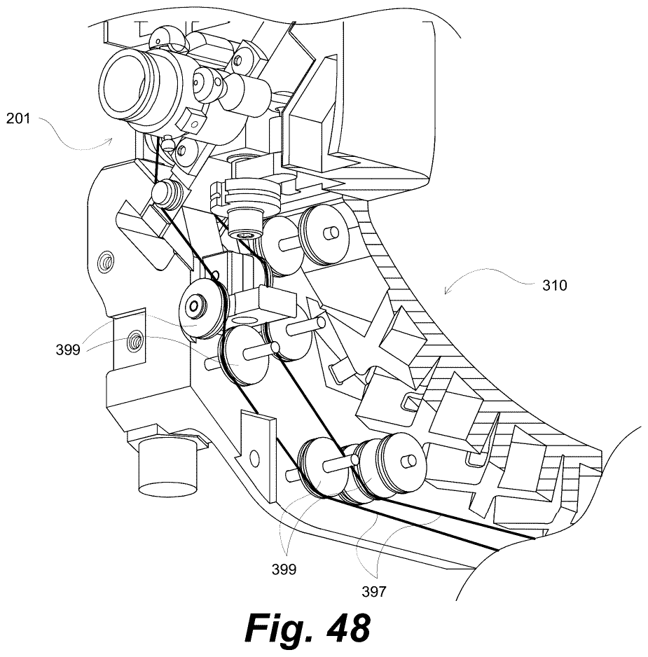

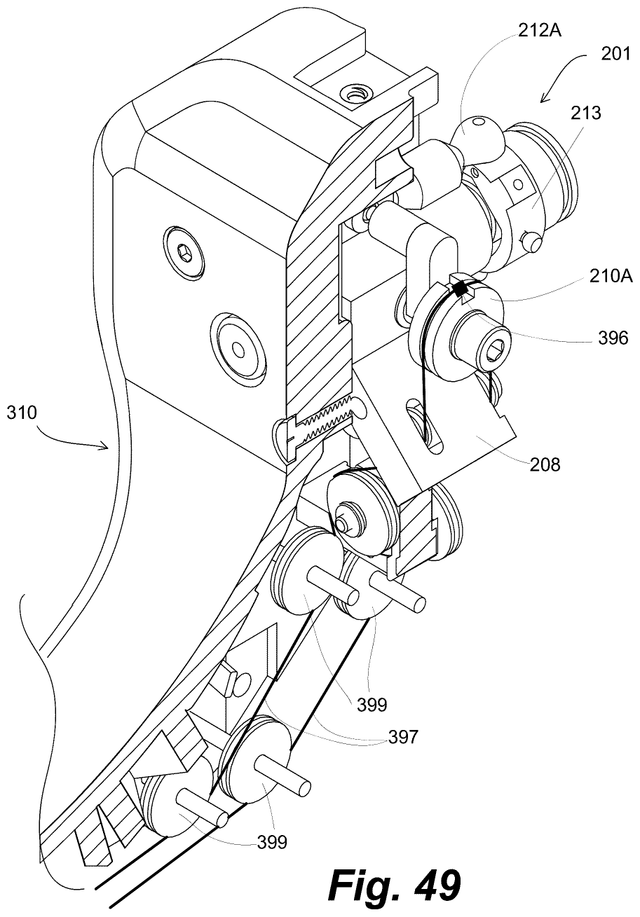

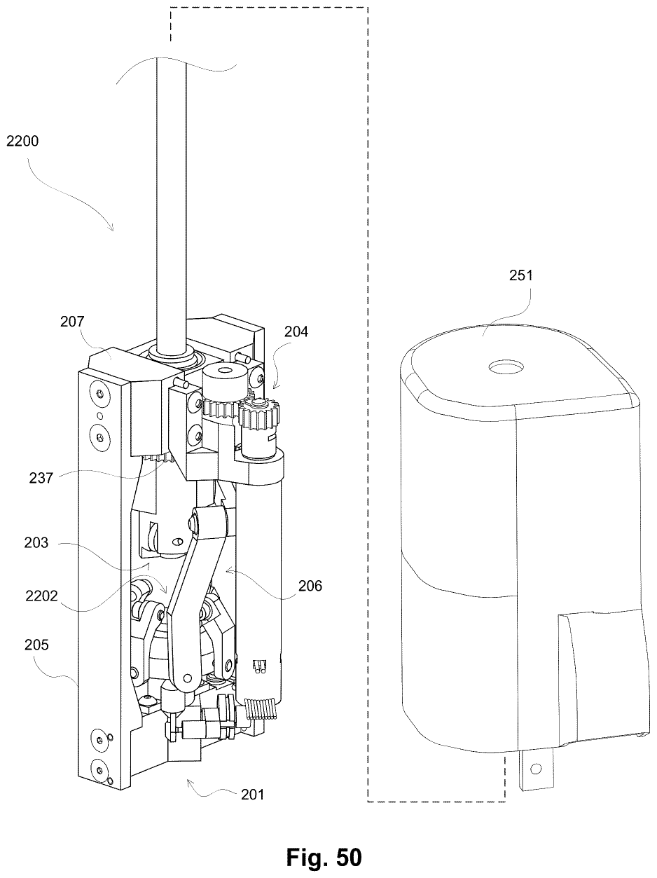

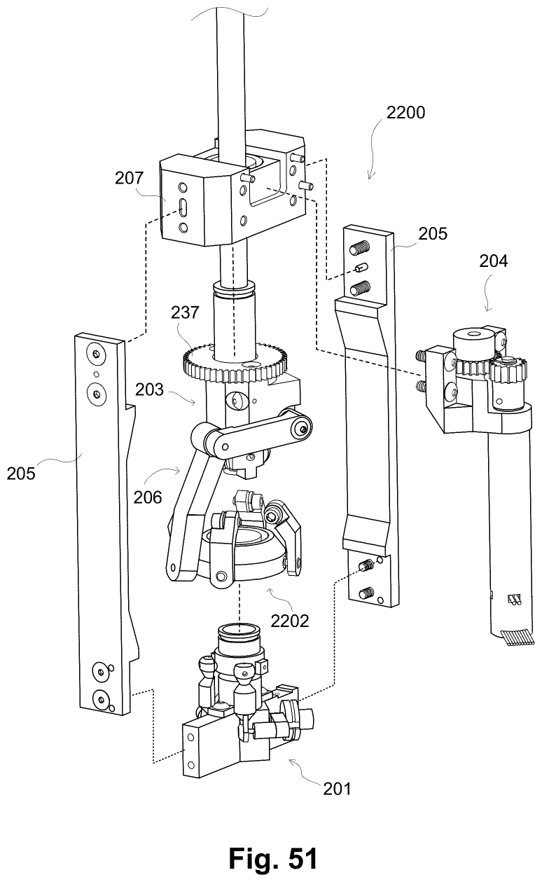

FIG. 46 and FIG. 47 depict the Pitch Input Cable 397 routed through the Input Linkage Assembly 300. FIG. 48 (looking from the distal end) and FIG. 49 (looking from the proximal end) depict the interface of the Pitch Input Cable 397) with the Articulation Subassembly 201.

FIGS. 46-49 show how the rotation of the Pitch Driving Pulley 372, and therefore the Pitch Link 381 and Pitch Joint Shaft 373, about the Pitch Input Axis A-A with respect to the Yaw Link Subassembly 350 (FIG. 46 and FIG. 47) is transmitted via the Pitch Input cable 397 to the Pitch Driven Pulley 210A via positive engagement between a Medial Crimp 396 mounted on the Pitch Input Cable 397 and a groove in the Pitch Driven Pulley 210A (FIG. 49).

The Pitch Driven Pulley 210A is located in the Swashbox Assembly 2200 at the distal end of the Chassis Subassembly 310. The rotation of the Pitch Driven Pulley 210A about Pitch Input Axis A-A acts as the input to the Swashbox Assembly 2200 corresponding to the Handle Assembly's 400 rotation about the Pitch Input Axis A-A (see FIG. 32) with respect to the Yaw Link Subassembly 350 and Chassis Subassembly 310.

Shown in FIG. 46-FIG. 49, to suitably route the Pitch Input Cable 397 along the Yaw Link 351 and Chassis 301, and to minimize frictional losses, Redirect Pulleys 399 mounted within the Yaw Link Subassembly 350, Yaw Joint Subassembly 320 and Chassis Subassembly 310 may be used. These Redirect Pulleys (also known as Idler Pulleys) are allowed to spin freely on Idler Shafts rigidly mounted to the Chassis 301, Yaw Joint Shaft 323 and Yaw Link 351. These Redirect Pulleys 399 are placed in such a way that the cable bend angles and cable path are minimized. The materials of these Idler Pulleys 399 and Idler Shafts are selected such that the friction between the Idler Pulleys 399 and the Idler Shafts is minimized.

Referring to FIG. 46, it is also important to note that the Pitch Input Cable 397 is routed through the center of the Yaw Joint Shaft 323. The risk of the motion of the Pitch Input Cable 397 being influenced by the rotation of the Yaw Link Subassembly 350 with respect to the Chassis Subassembly 310 about the Yaw Input Axis B-B is mitigated by centering the Pitch Input Cable 397 on the Yaw Input Axis B-B.

Referring back to FIG. 39, in some instances it may be desirable to limit the range of rotation of the Pitch Link Subassembly 380 with respect to the Yaw Link Subassembly 350 about the Pitch Input Axis A-A to avoid stressing the elements/components of the Pitch Transmission Subassembly. For example, failure to limit the aforementioned range of rotation in this situation can result in unnecessary stress on the Pitch Input Cable 397 and the rest of the Pitch Transmission Subassembly. To mitigate this risk, a Pitch Hardstop 382 is mounted to the Pitch Link 381. The Pitch Hardstop 382 makes positive contact with a boss that lies on the Yaw Link 351 when the range of rotation is met in either direction. This is simply one location for such a range limiting feature. Pitch Hardstops 382 can be located anywhere else along the Pitch Transmission Subassembly.

To ensure that the rotation of the Handle Assembly 400 with respect to the Yaw Link Subassembly 350 about the Pitch Input Axis A-A and the rotation of the Handle Assembly 400 with respect to the Chassis Subassembly 310 about Yaw Input Axis B-B (as shown in FIG. 32) is transmitted to the Swashbox Assembly 2200 with minimal losses, it is important to make the structural components of the Pitch Link Subassembly 380, Yaw Link Subassembly 350, and Chassis Subassembly 310 as rigid as possible. If these assemblies are too flexible, i.e. not rigid enough, the rotations of the Handle will not be fully transmitted via the Yaw Input Cable 398 and the Pitch Input Cable 397 resulting in a perceived compliant behavior (i.e. lacking in transmission stiffness) of the Distal Manipulator 100 articulation in response to the input articulation at the Handle Assembly 400. To make the aforementioned assemblies sufficiently rigid in loading that is expected from rotations of the Handle Assembly, cross ribbing is added to the Pitch Link 381, Yaw Link 351, and Chassis 301. In a preferred embodiment, the cross ribbing geometry resembles an “X”, a straight beam, or a combination of the two. Other possible embodiments are octagonal or hexagonal beams, i.e. beams resembling a “honeycomb-like” geometry or adjacent hexagons or octagons. Triangular beams structured in a likewise manner is another example of such stiffening features.

In the preferred embodiment, the Device provides the user with haptic feedback to the user. Haptic feedback for the articulation motions (Yaw and Pitch) allows the user to apply more precise inputs, since they receive feedback about how their inputs are affecting the object that the Distal Manipulator 100 is acting upon. This haptic feedback can be used by the user, such that they can adjust their inputs, without the need for visual feedback, in order to achieve more favorable interaction between the Distal Manipulator 100 and the object that it is acting upon.

In the preferred embodiment, this is achieved by the Handle Assembly 400 being rigidly connected to the Input Linkage Assembly 300 via the Pitch Link Interface 410. These structural features along with the yaw and pitch transmission subassemblies and mechanisms in the overall Device &&& allow the forces, torques, and vibrations experienced by the Distal Manipulator 100 to reach the Pitch Link 381, where they can then pass from the Pitch Link 381 into the Handle Body Subassembly 460, and then subsequently be felt by the user's hand as they grasp the Handle Body Subassembly 460.

Referring to FIG. 32, the purpose of the Swashbox Assembly 2200 is to receive two independent articulation inputs (i.e. rotations about the Yaw Input Axis B-B and Pitch Input Axis A-A) from the Handle Assembly 400, along with the Roll input from the Dial Subassembly 430 (see FIG. 34). The Swashbox Assembly 2200 translates rotation about the Yaw Input Axis B-B, Pitch Input Axis A-A, and Roll Input Axis C-C into rotation of the Distal Manipulator 100 about the Yaw Output Axis Y-Y, Pitch Output Axis X-X, and Roll Output Axis Z-Z. The Swashbox Assembly ensures that the translation of these rotational motions is a decoupled process. In other words, any of the three inputs (rotation about the Yaw Input Axis B-B, Pitch Input Axis A-A, and Roll InputAxis C-C) can be applied or changed while the other two remain unaffected. The method in which the Swashbox Assembly 2200 achieves this is described in the following sections.

Referring to FIGS. 50 and 51, one embodiment of the Swashbox Assembly 2200 is shown. FIG. 50 shows an assembled view of the Swashbox Assembly 2200 with the Swashbox Housing 251 shown on the side; while FIG. 51 shows an exploded view of this assembly with all of its sub-assemblies intact but without the Swashbox Housing 251.

The Swashbox Assembly 2200 comprises several sub-assemblies which are defined as the following. An Articulation Subassembly 201 which couples to a Rotating Plate Subassembly 2202 through a bearing interface. A Tool Shaft Subassembly 203 that is pressed into a Main Bearing Housing 207 on the distal and is coupled to a Rotating Plate Subassembly 2202 via a Coupler Linkage 206 (also referred to as the Roll Drive Coupler). A Roll Transmission Subassembly 204 is mounted to the Main Bearing Housing 207 and meshes with a Third Gear 237 in the Tool Shaft Subassembly 203. Also included are Structural Spines 205, which are mounted to the Main Bearing Housing 207 on the distal end and the Articulation Subassembly 201 on the proximal end via screw joints. The Swashbox Housing 251 is then placed over the Swashbox Assembly 2200. These subassemblies are explained in further detail in the following sections.

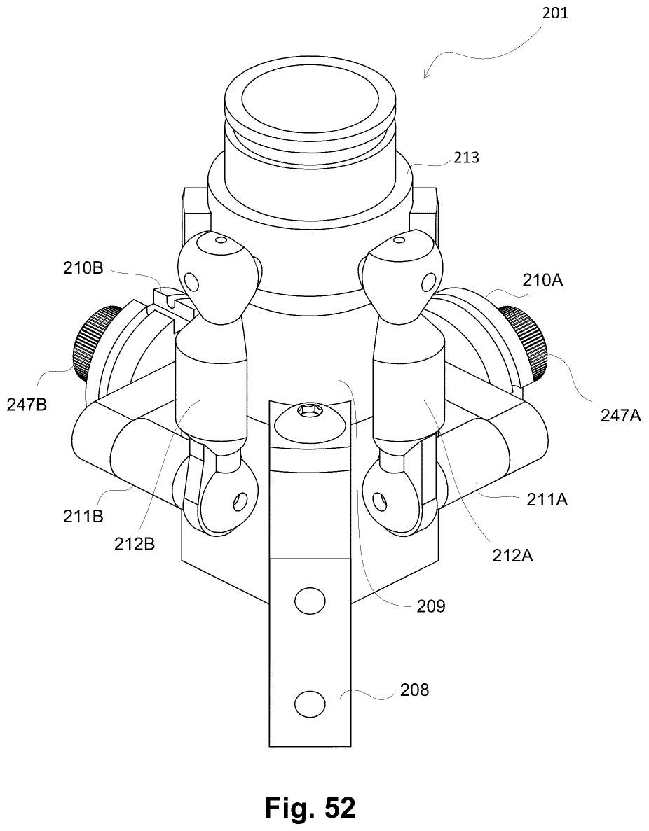

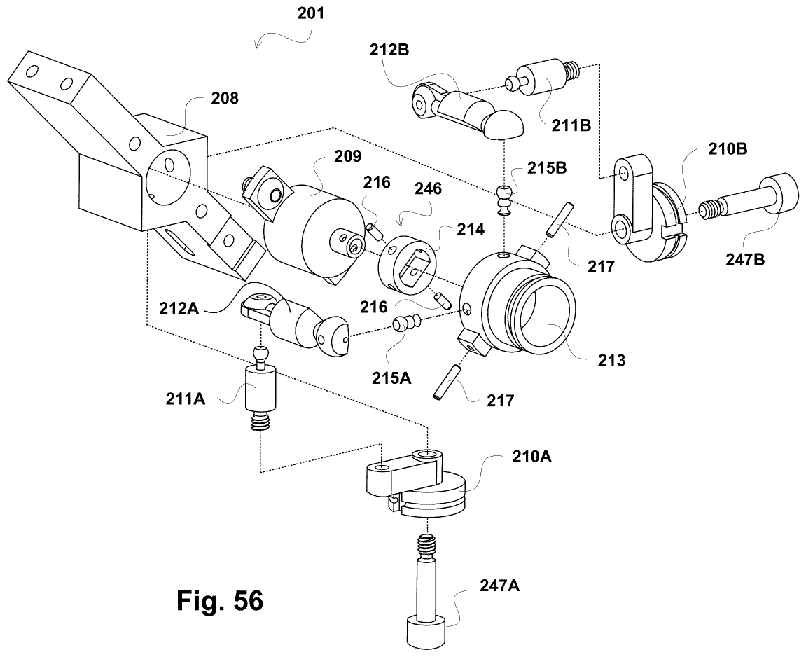

Referring to FIGS. 52 and 56, an assembled view of the Articulation Subassembly 201 is shown in FIG. 52 and an exploded view of the Articulation Subassembly 201 is shown in FIG. 56. The Articulation Subassembly 201 receives two inputs (motions due to rotations about the Yaw Input Axis B-B and Pitch Input Axis A-A rotations), which are sent from the Handle Assembly 400 through the Input Linkage Assembly 300 via the Pitch 397 and Yaw 398 Cables (shown in FIG. 49 and FIG. 44, respectively). It then combines those two independent 1 DoF inputs and transmits the combined motion to the Non-rotating Plate 213 that is coupled to the Non-rotating Plate Ground 208 via a Gimbal Joint 246. These two 1 DoF inputs may be the rotations about the Pitch Input Axis A-A and Yaw Input Axis B-B generated by the user.

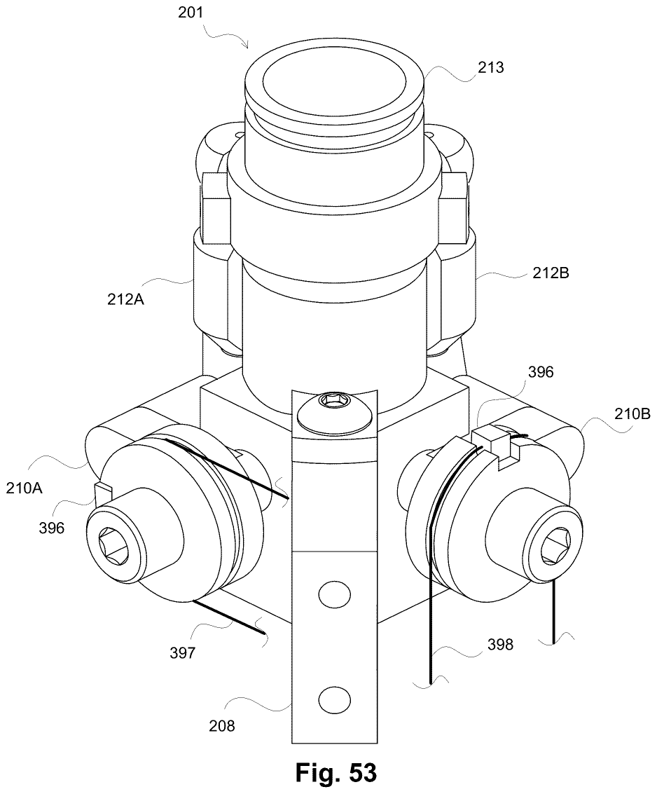

Referring to FIG. 53, another view of the assembled Articulation Subassembly 201 is shown. One preferred embodiment of the Articulation Subassembly 201 consists of a Yaw Driven Pulley 210B and a Pitch Driven Pulley 210A. These pulleys receive motion due to rotations about the Yaw Input Axis B-B and Pitch Input Axis A-A rotations via the Yaw Cable 398 and Pitch Cable 397, respectively.

Referring now to FIGS. 52 and 56, the Yaw Driven Pulley 210B and Pitch Driven Pulley 210A further transfer their respective rotations through their corresponding mechanisms to the Non-rotating Plate 213, causing it to rotate about axes D-D and E-E of the Gimbal Joint 246 with respect to Non-rotating Plate Shaft 209.

The Pitch drive linkage comprises the Pitch Driven Pulley 210A and a Ball Stud Standoff 211A which are rigidly interconnected with a screw joint. The Pitch drive linkage also includes the Pitch Driven Link 212A, a joint 215A comprising a ball stud, and the Non-rotating Plate Shaft 209 which is rigidly attached to the Non-rotating Plate Ground 208 together via a screw joint. The Pitch Driven Pulley 210A is mounted to the Non-rotating Plate Ground 208 with a Shoulder Screw 247A which acts as both a shaft for the pulley to rotate about and as an axial constraint. The Pitch Driven Link 212A couples the Pitch Driven Pulley 210A to the Non-rotating Plate 213 via the joint 215A and Ball Stud Standoff 211A.

The Yaw drive linkage comprising the Yaw Driven Pulley 210B and Yaw Driven Link 212B, among other elements, has a similar configuration but in a different plane as shown in FIGS. 52 and 56.

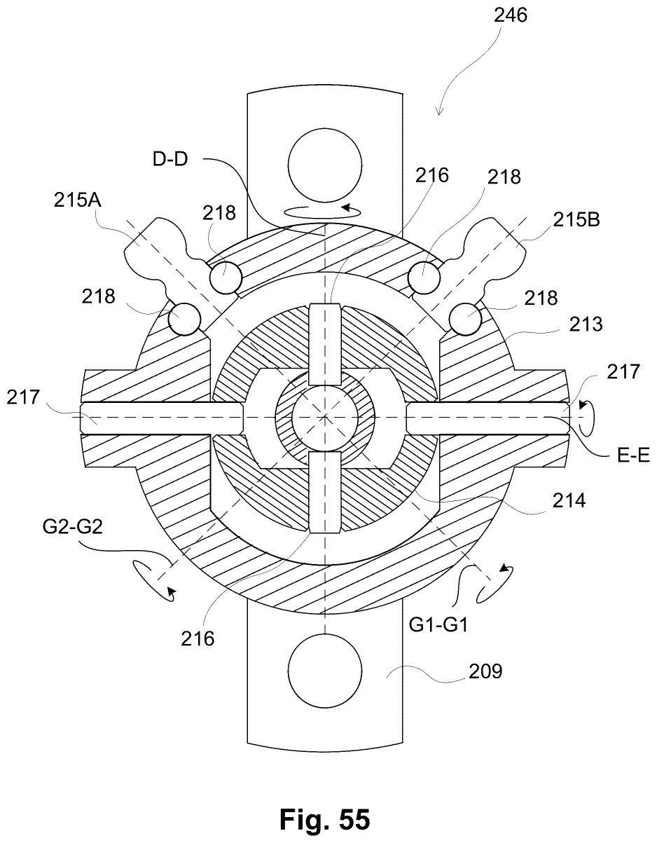

Referring to FIG. 55, a cross section of the Gimbal Joint 246 (which is part of the Articulation Subassembly 201) is shown. The Gimbal Joint 246 consists of the Non-rotating Plate Shaft 209 in the center, the Inner Ring 214, and an outer ring (i.e. Non-rotating Plate) 213 which are sequentially coupled to each other with a set of Short Pins 216 and Long Pins 217. The Inner Ring 214 rotates with respect to the Non-rotating Plate Shaft 209 about axis D-D, while the Non-rotating Plate 213 rotates with respect to the Inner Ring 214 about axis E-E.

Referring to FIG. 53, the Articulation Subassembly 201 receives motion, due to rotations about the Yaw Input Axis B-B and Pitch Input Axis A-A rotations, from the Handle Assembly 400 through the Input Linkage Assembly 300 via the Pitch Input Cable 397 and Yaw Input Cable 398. This allows the Articulation Subassembly 201 to transfer motion to the corresponding Yaw Output Axis Y-Y and Pitch Output Axis X-X at the Distal Manipulator 100, such that the transmission of rotational motion from the Handle Assembly 400 is consistent in all directions at the Distal Manipulator 100. In the preferred embodiment, this is done with the Yaw Driven Pulley 210B and Pitch Driven Pulley 210A which are orthogonal to one another. Since they are orthogonal and the transmission ratio provided by the Yaw Transmission Subassembly and Pitch Transmission Subassembly may be approximately equal, therefore the transmission ratio from the Handle Assembly 400 to the Non-rotating Plate 213 generally remains uniform in all articulation directions (i.e. various combinations of rotations about axes G1-G1 and G2-G2). In order to receive the motion generated by rotation of the Handle Assembly 400 about the Yaw Input Axis B-B and the Pitch Input Axis A-A, the Yaw Cable 398 and Pitch Cable 397 terminate at their respective Driven Pulleys 210B and 210A via respective Medial Crimps 396, coupling the rotation of the Driven Pulleys 210B and 210A to the translation of the respective cables. In an alternative embodiment this can be transmitted electronically by attaching motors directly to the Driven Pulleys 210B and 210A, pneumatically or hydraulically with a pneumatic or hydraulic rotary actuation, or the Yaw 398 and Pitch 397 Cables could directly attach to the Non-rotating Plate 213.

Referring to FIG. 52 and FIG. 53, once the motions about the Yaw Input Axis B-B and the Pitch Input Axis A-A are received by the Yaw Driven Pulley 210B and Pitch Driven Pulley 210B, they are separately and independently transferred to the Non-rotating Plate 213, causing the Non-rotating plate 213 to rotate about axes G1-G1 and G2-G2. In the current embodiment, these rotations are transferred from the Yaw Driven Pulley 210B and Pitch Driven Pulley 210A to the Non-rotating Plate 213 through the Yaw Driven Link 212B and Pitch Driven Link 212A.

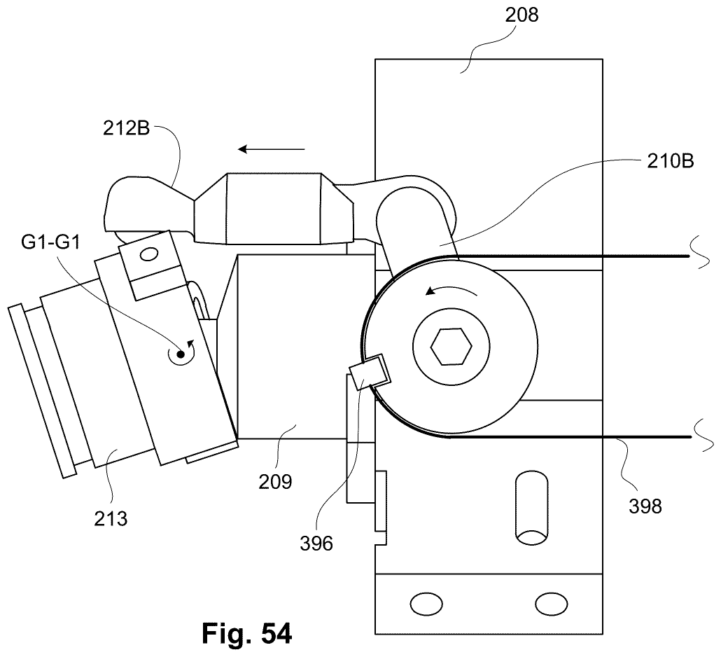

Referring now to FIG. 54, which shows a side view of the Articulation Subassembly 201 normal to the Yaw Driven Pulley 210B. When the Yaw Driven Pulley 210B rotates with respect to the Non-rotating Plate Ground 208 due to the Yaw Cable 398, it imparts an approximately linear motion along the Yaw Driven Link 212B which in turn causes the Non-rotating Plate 213 to rotate around its center about axis G1-G1. The same process occurs for Pitch rotation transmission, but in a different plane.

Referring to FIG. 55 and FIG. 56, the joint 215A that connects the Pitch Driven Link 212A to Non-rotating Plate 213, and the joint 215B that connects the Yaw Driven Link 212B and to Non-rotating Plate 213, should provide at least 2 DoF. These joints could be universal, cardan, spherical, or ball and socket joints, or any other type of joint or combination of joints that allows two DoF.

In the present embodiment, joints 215A and 215B are based on ball studs. The center of rotation of these joints lie in plane DE, which is the plane that contains axes D-D and E-E of the Gimbal Joint 246. This configuration allows rotational motion about axes G1-G1 and G2-G2 to be separately and independently transferred from the Pitch Driven Pulley 210A and Yaw Driven Pulley 210B to the Non-rotating Plate 213 without hindering the movement of one another; meaning these rotations about these axes are decoupled. In an alternative embodiment, this joint could be accomplished by directly attaching the Yaw Input Cable 398 and Pitch Input Cable 397 to the Non-rotating Plate 213 as aforementioned, or hydraulically by utilizing pistons which tip/tilt the Non-rotating Plate 213, or electronically with motors attached to ball/lead screws with a spherical joints at the end.

Referring to FIG. 55, when the rotations about the Pitch Input Axis A-A and Yaw Input Axis B-B are transferred through the Pitch Driven Link 212A and Yaw Driven Link 212B, they separately act upon the Gimbal Joint 246 causing the Non-rotating Plate 213 to rotate about axes G1-G1 and G2-G2. In order to make it such that rotations about the Pitch Input Axis A-A and the Yaw Input Axis B-B from the Input Linkage Assembly 300 remain decoupled in the Non-rotating Plate 213, the 2 DoF joint must allow for independent rotation of two axes. In the current embodiment, this is achieved with the Gimbal Joint 246 as shown in FIG. 55. The Gimbal Joint 246 is designed such that its 2 DoF, represented with axes D-D and E-E, intersect in the center. This results in the rotation about one axis not impacting the location of the second axis in space, therefore making them decoupled. In addition, the DoF of the spherical joint represented with axes G1-G1 and G2-G2 must also intersect with axes D-D and E-E as shown in FIG. 55, so that the movement of one linkage does not cause unwanted movement in the other. In an alternative embodiment, the 2 DoF joint could be a spherical joint, traditional U joint, or other joint which achieves the aforementioned requirements of at least 2 Rotational DoFs.

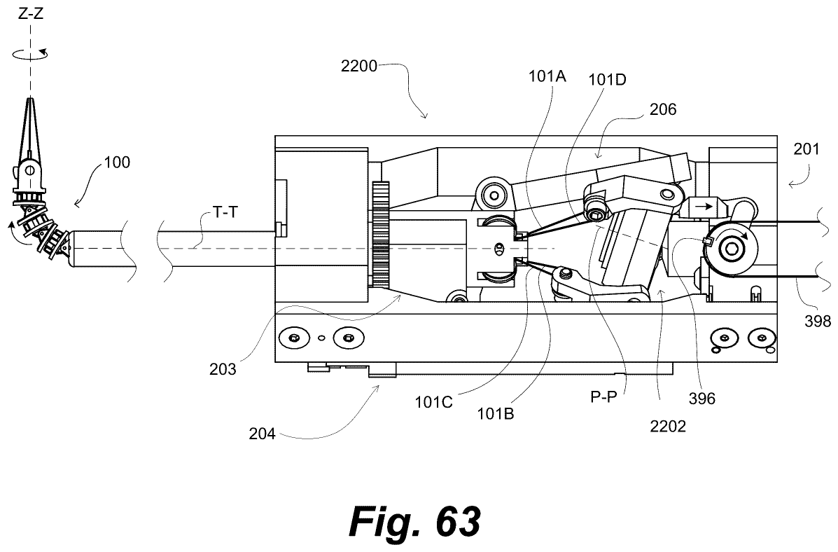

Referring to FIG. 63, a view that shows the Swashbox Assembly 2200, and Distal Manipulator Assembly 100 in an articulated position is shown. The Rotating Plate Subassembly 2202 transmits rotation about axes G1-G1 and G2-G2, from the Articulation Subassembly 201, to the Distal Manipulator Assembly 100 via the Tool Shaft Subassembly 203. The Rotating Plate Subassembly 2202 has been designed to allow roll rotation about the articulated axis P-P with respect to the Articulation Subassembly 201, while also transmitting the rotation about axes G1-G1 and G2-G2 from the Articulation Subassembly 201 to the Rotating Plate Subassembly 2202.

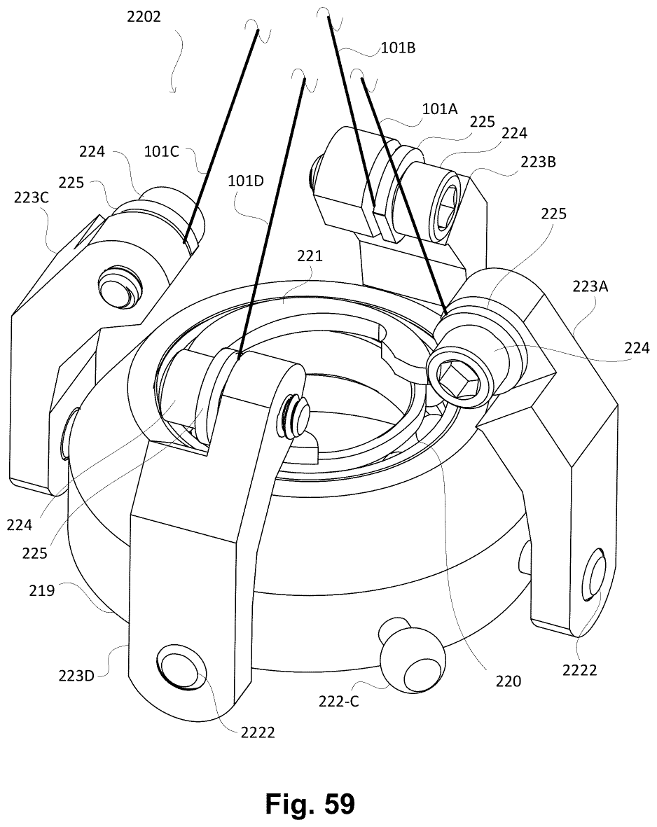

Referring to FIG. 59, in the preferred embodiment, the Rotating Plate Subassembly 2202 consists of the Rotating Plate 219, Rotating Plate Bearing 220, Snap-ring 221, Coupler Link Ball Stud joint 222-C, and four Cable Legs 223A through 223D each coupled to the Rotating Plate 219 via a respective Ball Stud 2222. In the current view, the respective Ball Studs 2222 for Cable Legs 223A and 223D can be seen, while those for 223B and 223C are hidden. In addition, for each of the Cable Legs 223A-D they contain their respective Clamping Screw 224 and Clamping Washer 225.

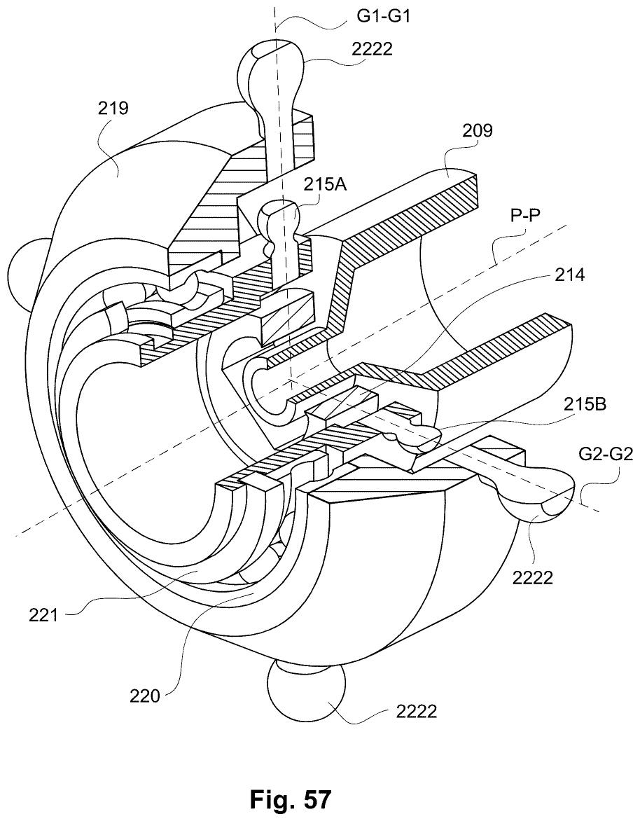

Referring to FIG. 57, a quarter cross section of the interface between the Gimbal Joint 246, and the Rotating Plate 219, Rotating Plate Bearing 220 and Snap-ring 221 is shown. The Rotating Plate 219 is attached to the Non-rotating Plate 213 via the Rotating Plate Bearing 220 secured with a Snap-ring 221. This mates the Rotating Plate Subassembly 2202 to the Articulation Subassembly 201 as seen in FIG. 50. This allows the Rotating Plate 219 to rotate with respect to the Non-rotating Plate 213 about axis P-P. Referring to FIG. 59, each of the Cable Legs 223A-D are attached to the Rotating plate 219 via a snap fit to their respective Ball Studs 2222 which are pressed onto the Rotating plate 219. In addition, the Coupler Link Ball Stud joint 222-C is also pressed onto the Rotating plate 219 and is attached to the Coupler Linkage 206 via a snap fit as shown in FIG. 51. Referring now to FIG. 57, rotation about axes G1-G1 and G2-G2 is transferred from the Articulation Subassembly 201 to the Rotating Plate Assembly 2202, through the Non-rotating Plate 213 to the Rotating Plate 219 since they are coupled to one another. This means that when the Non-rotating Plate 213 tips/tilts about axes D-D and E-E due to the rotations from the Driven Pulleys 210 about axes G1-G1 and G2-G2, the Rotating Plate 219 inherits this tip/tilt motion.

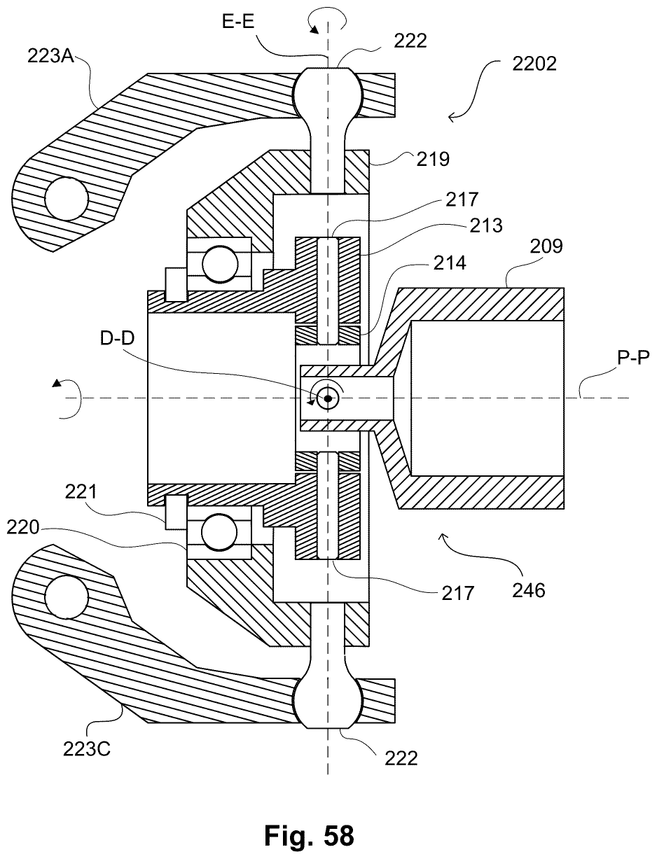

Referring to FIG. 63, the Rotating Plate Subassembly 2202 translates the rotational motion about axes G1-G1 and G2-G2 of the Articulation Subassembly 201 to the Distal Manipulator 100 by actuating the Distal Manipulator Cables 101A-D in such a way that they are decoupled. The following now refers to FIG. 58, which shows a cross section of the interface between the Gimbal Joint 246, and the Rotating Plate 219, Rotating Plate Bearing 220 and Snap-ring 221. This decoupling is achieved by ensuring that the rotational center of the joints that attach the Distal Manipulator Cables 101A-D to the Rotating Plate Assembly 202 lie on plane DE. This is achieved by attaching the Distal Manipulator Cables 101A-D to the Rotating Plate 219 with a spherical joint, and then by overlapping the Rotating Plate 219 and Non-rotating plate 213 so that the center of the spherical joint is coincident with plane D-E. In the preferred embodiment, the Rotating plate 219 is overlapped over the Non-rotating plate 213. Referring now also to FIG. 58, in an alternative embodiment, the decoupling of rotational motion about axes G1-G1 and G2-G2 can also be achieved by designing the Non-rotating Plate 213 to overlap over the Rotating Plate 219, by having both plates lie in plane DE, or some other variation which ensures that the rotational center of the joints that attach the Distal Manipulator Cables 101A-D to the Rotating Plate Subassembly 2202 lie on plane DE.

Referring to FIG. 59, in the current embodiment, the Distal Manipulator Cables 101A-D are attached to their respective Cable Legs 223A-D via a clamping interface, which are then attached to the Rotating plate 219 via spherical joints. In alternative embodiments of the Cable Legs 223A-D, The joint that attaches it to the Rotating plate 219 could be a U joint or other 2DoF joint, and the attachment point of the Distal Manipulator Cables 101A-D could be a crimp interface.

Referring to FIG. 59, the preferred embodiment of securing the Distal Manipulator Cables 101A-D to the Cable Legs 223A-D is through a clamping interface. This is done by clamping the Distal Manipulator cables 101A-D between a plastic surface which comes from the Cable Leg 223A-D, and a metal surface which comes from their respective Clamping Washer 225, while their respective Clamping Screw 224 maintains the pressure on the Distal Manipulator Cables 101A-D that they experiences between the two surfaces. This could also be done between two metal surfaces or two plastic surfaces, however a metal and plastic surface is preferred to maintain clamping pressure while not causing damage to a cable.

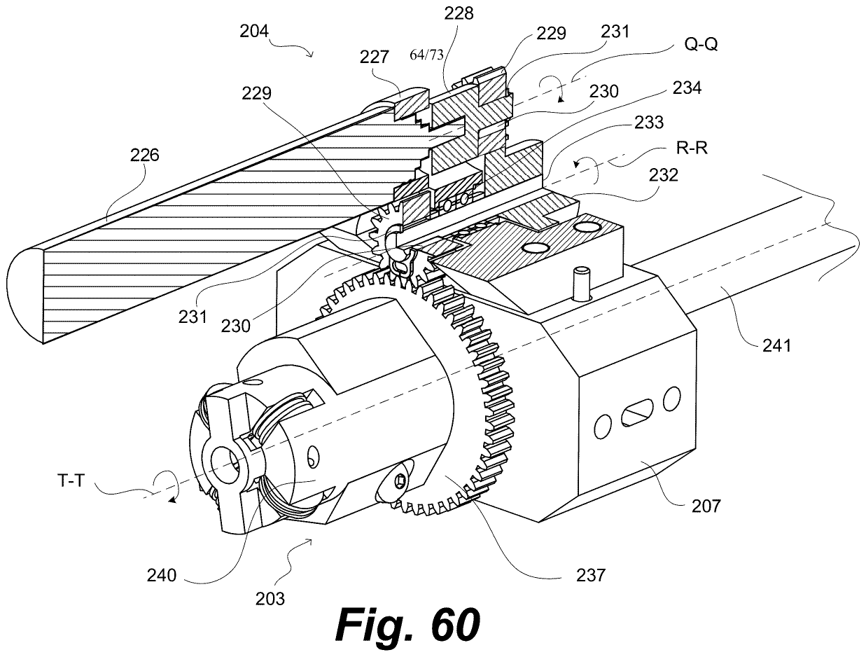

This Subassembly receives (via the Mechatronics System 700) an input that corresponds to the rotational motion that the user imparts into the Dial Subassembly 430 about Roll Input Axis C-C as shown in FIG. 33. That input is then converted into rotational motion that is imparted onto the Tool Shaft 241 through the Roll transmission Subassembly 204 as shown in FIG. 60, which shows a quarter cross section of the Roll Transmission Subassembly 204, with the Tool Shaft Subassembly 203.

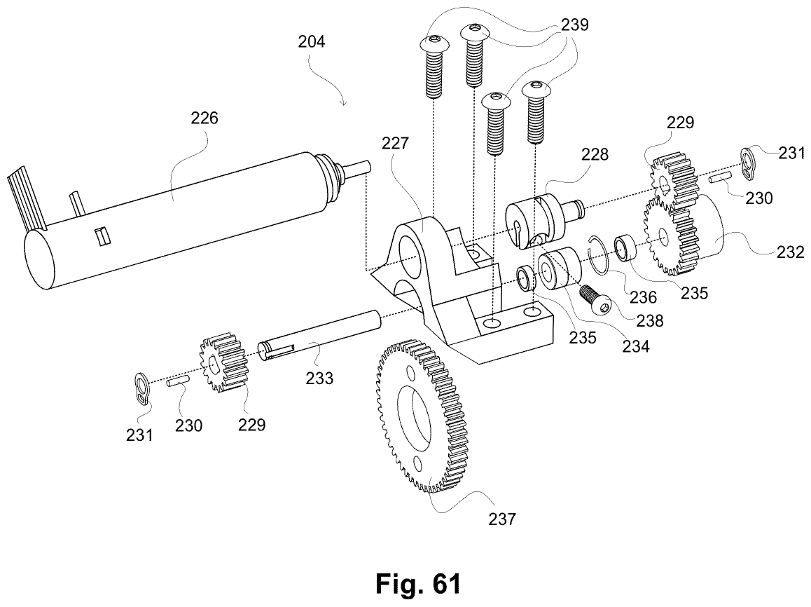

In this preferred embodiment, the Motor 226 receives a command from the Mechatronic System 700. The command dictates that the Motor 226 changes the angular position of its output shaft about axis Q-Q, which directly correlates to the rotational motion that the Mechatronic System 700 measured from the Dial Encoder 761 seen in FIG. 36 as a result of the user changing the Dial's 431 angular position about Roll Input Axis C-C. Referring to FIG. 60 and FIG. 61, which shows an exploded view of the Roll Transmission Subassembly 204, the rotation of the Motor 226 output shaft is directly transferred via a clamping collar joint to the Motor Collar 228, to which the First Gear 229 is attached with a Dutchman Pin 230 and Gear Snapring 231 joint. The First Gear 229 meshes with Second Gear 232 to transfer the motion to Intermediate Shaft 233 to which it is mounted via a clamping collar joint. Intermediate Shaft 233 is supported and allowed to rotate by Intermediate Bearing 234, which it passes through. Intermediate Bearing 234 is housed in the Intermediate Housing 227, which is mounted to the Main Bearing Housing 207 via a screw joint. Intermediate Shaft 233 transfers the motion to a second First Gear 229 which is mounted to it with a Dutchman Pin 230 and Gear Snapring 231 joint. The First Gear 229 meshes with Third Gear 237 in order to transfer the motion to the Tool Shaft Sleeve 240, which is part of the Tool Shaft Subassembly 203. The Third Gear 237 is mounted to the Tool Shaft Sleeve 240 via a screw joint.

Referring to FIG. 60, in the preferred embodiment, the Motor 226 applies torque to the Tool Shaft 241 which results in a desired change in its angular position about axis T-T. The Motor 226 is connected to the Tool Shaft 241 mechanically with a geared reduction, which increases the torque and decreases the rotational speed at the Tool Shaft 241 with respect to the Motor 226 output shaft. In alternative embodiments, this connection may also be achieved by a belt drive, friction drive, chain drive, cable drive, or without a geared reduction, and could also utilize a magnetic, electromagnetic, or fluid coupling as a means of transferring the torque from the Motor 226 to the Tool Shaft 241. In any of the aforementioned alternative embodiments, a sufficient amount of torque is applied to the Tool Shaft 241 such that angular position of a Tool Shaft 241 is related to the angular position of the Dial 431 by a constant ratio, even when there is a reasonable resistive torque applied to the Tool Shaft 241 as a result of the Device being used for actions such as the Distal Manipulator providing a torque at the output in the roll direction. The ratio for any given embodiment could be any magnitude, such as 1:1 in one instance, and that ratio will be maintained as the Roll Transmission Subassembly 204 changes Tool Shaft's 241 angular position and applies a torque to an object being manipulated.

Referring to FIG. 60, in the preferred embodiment, the Device is designed to have a specific ratio between the change in Dial 431, seen in FIG. 35, angular position and the resulting change in Tool Shaft 241 angular position. There is also a constant ratio between the angular position of the Motor 226 armature and the Tool Shaft 241, which is defined by geared transmissions both internal and external to Motor 226, and may or may not be the same ratio as the one between the angular position of the Dial 431 and the angular position of the Tool Shaft 241. The ratio of the angular positions of the Motor 226 armature and the Tool Shaft 241 is selected such that the Motor 226 is able to operate at an ideal operating point, which could be selected to provide the user with optimal torque at the Tool Shaft 241, optimal angular speed at the Tool Shaft 241, optimal power efficiency for the Motor 226, a combination of the former, or any other desired operating point, all while contributing to the maintenance of the constant ratio between the angular positions of the Tool Shaft 241 and Dial 431.

Referring to FIG. 60, in the preferred embodiment, the Roll Transmission Subassembly 204 is not only designed to be able to maintain the constant ratio between the angular positions of the Dial 431, seen in FIG. 35, and the Tool Shaft 241 while the user is commanding a change in angular position, but also maintains the positional relationship when no command is given by the user to change angular position. The Roll Transmission Subassembly 204 is designed to resist the attempts of external torques to change the angular position of the Tool Shaft 241. The preferred embodiment achieves this by using the Motor 226 to apply an opposing torque to the rest of the Roll Transmission Subassembly 204 such that a torque is applied to the Tool Shaft 241 of equal magnitude to the external load, thus resulting in no net torque on the Tool Shaft 241, and therefore no change in the angular position of the Tool Shaft 241. In other embodiments, this functionality can also be achieved by mechanical, magnetic, pneumatic, or hydraulic interlocks that constrain the rotational degree of freedom of any of the components in the Roll Transmission Subassembly 204, depending on whether or not the user is changing the angular position of the Dial 431.

Referring to FIG. 60, The Roll Transmission Subassembly 204 is designed in such a way that, if excessive torque is applied to the Roll Transmission Subassembly 204, it has the ability to slip in angular position before any component in the transmission fails mechanically. The ability to do this preserves the mechanical integrity of the Roll Transmission Subassembly 204 throughout the application of the excessive torque, such that it may return to desired functionality after the excessive torque is relieved. In the preferred embodiment, this is achieved by intentionally limiting the amount current supplied to the Motor 226, and thus limiting the amount of torque that the Motor 226 can apply to the rest of the Roll Transmission Subassembly 204. If the opposing external torque is higher than this limit, then the Motor 226 armature will rotate inside the Motor 226 housing in order to prevent further external torque from being applied to the Roll Transmission Subassembly 204 or Tool Shaft Subassembly 203. If the user is actively applying a rotational motion to the Dial 431, they will be able to continue doing this until the excessive external load is relieved, due to the mechatronic interface between the Dial Subassembly 430 and the Roll Transmission Subassembly 204, which allows for slip as described above. Once the external load is relieved, the rotational motion of the Tool Shaft Subassembly 203 will resume its relationship with the Dial 431 rotational motion. In alternative embodiments, this functionality could be achieved at any interface by implementing mechanical clutches, friction couplings, fluid couplings, magnetic couplings, or any other couplings or mechanisms that allow for components to slip relative to each other after a predefined torque threshold has been breached.

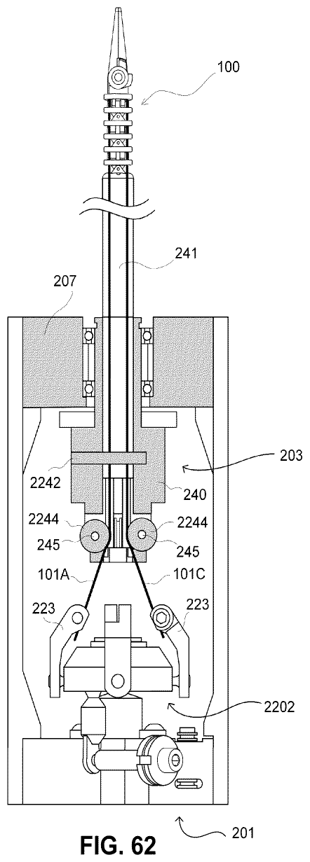

Referring to FIG. 62, a cross section of the Tool Shaft Subassembly 203 and Main Bearing Housing 207 with the Rotating Plate Subassembly 2202 and Articulation Subassembly 201 shown, is depicted. The Tool Shaft Subassembly 203 acts as a conduit which transmits rotation from the Rotating Plate Subassembly 2702 about axes G1-G1 and G2-G2, and rotation about axis R-R from the Roll Transmission Subassembly 204 as seen in FIG. 60, then transfers those motions to the Yaw Output Axis Y-Y, Pitch Output Axis X-X and Roll Output Axis Z-Z at the Distal Manipulator Assembly 100 (see FIG. 32).

Referring to FIG. 62, the preferred embodiment of the Tool Shaft Subassembly 203 consists of a Tool Shaft 241, Tool Shaft Sleeve 240, Cross Pin 2242, Tool Shaft Redirect Pulleys 2244, and Pulley Pins 245 for each pulley. The Distal Manipulator Assembly 100 seats on top of the Tool Shaft 241, held in place by the tension in the Distal Manipulator Cables 101A-D. The Distal Manipulator Cables 101A-D run through the Tool Shaft 241, ride the four Tool Shaft Redirect Pulleys 2244 attached to the Tool Shaft Sleeve 240 with Pulley Pins 245, and terminate at the clamping interface of their respective Cable Legs 223A-D. The Tool Shaft Sleeve 240 is pressed onto the proximal end of the Tool Shaft 241, and rigidly attached to it with a Cross Pin 2242 which is secured to the Tool Shaft Sleeve 240 with a screw joint.

Referring to FIG. 62, the Tool Shaft 241 receives motion from the Rotating Plate Subassembly 2202, then redirects that motion by using the Tool Shaft Redirect Pulleys 2244 to route the Distal Manipulator Cables 101A-D from the Rotating Plate Assembly 2202 to the Distal Manipulator 100. It is important that the Tool Shaft Redirect Pulleys 2244 be made of a lubricious material and rotate, as to reduce resistance to the movement of the Distal Manipulator Cables 101. As shown in FIG. 63, the Articulation Assembly 201 receives motion due to the rotation of the Handle Assembly 400 about the Yaw Input Axis B-B, then transmits that motion to the Rotating Plate Subassembly 2202. Since the Distal Manipulator Cables 101A-D are attached to the Rotating Plate 213, as it tilts, it pulls Distal Manipulator Cables 101A-D towards the proximal end of the Swashbox Assembly 2200. This produces a moment at each of the links in the Distal Manipulator 100 causing it to articulate in about the Yaw Output Axis Y-Y An analogous process occurs in order to produce rotation about the Pitch Output Axis X-X and in any combination of rotations about the Pitch Output Axis X-X and the Yaw Output Axis Y-Y.

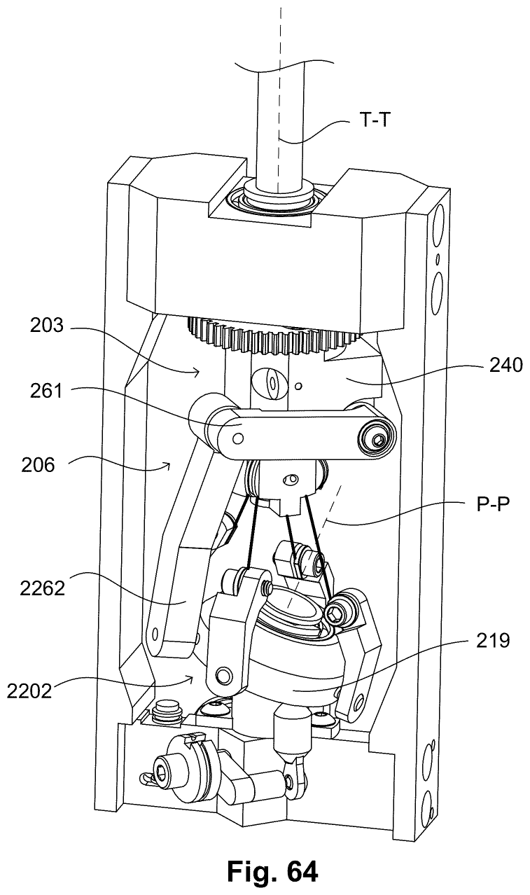

Referring to FIG. 64, in order for the Rotating Plate Subassembly 2202 to rotate about axes G1-G1 and G2-G2 while coupling the rotation of the Rotating Plate Subassembly 2202 about P-P to the rotation of the Tool Shaft Subassembly 203 about axis T-T, the Coupler Linkage 206 may be a R-R-S kinematic chain. In the current embodiment, the Coupler Linkage 206 comprises the Distal Link 261 and the Proximal Link 2262, which are attached together end-to-end by a revolute joint. The other end of the Distal Link 261 is attached to the Tool Shaft Sleeve 240 with a revolute joint, and the other end of the Proximal Link 2262 is attached to the Rotating Plate 219 via the Coupler Link Ball Stud joint 222-C (FIG. 59), which is a spherical joint. In an alternate embodiment, the Coupler Linkage 206 may be a R-R-U kinematic chain, with the last joint the chain being a universal (U) joint.

Referring to FIG. 60, The Tool Shaft 241 receives rotational motion from the Roll Transmission Subassembly 204 through the Third Gear 237. Referring now to FIG. 63, this causes rotation of the Tool Shaft Subassembly 203 about axis T-T which induces rotation of the Distal Manipulator 100 about Roll Output Axis Z-Z and the Rotating Plate Subassembly 2202 about axis P-P. The rotation of the Rotating Plate Subassembly 2202 about axis P-P is a result of the coupling of rotation between the Tool Shaft Subassembly 203 to the Rotating Plate Subassembly 2202 via the Coupler Linkage 206. This rotational coupling is essential to the continuous roll functionality of the Swashbox Assembly 2200; if the rotations were not coupled, the Distal Manipulator Cables 101A-D may twist around each other, and the transmission would bind.

Looking at FIG. 63, when the Distal Manipulator 100 rotates about the Yaw Output Axis Y-Y, the Distal Manipulator Cables 101A-D near the top side of the figure are pulled towards the proximal end of the Swashbox Assembly 2200. As the Rotating Plate Subassembly 2202 rolls about axis P-P, the positions of the Distal Manipulator Cables 101A-D changes as they orbit, but the Rotating Plate Subassembly 2202 remains at the same articulated angle. This ensures that whenever the Distal Manipulator Cables 101A-D rotate to the top position, they will be pulled towards the proximal end of the Swashbox Assembly 2200, allowing the Distal Manipulator 100 to maintain roll rotation at the same articulated angle.

In many applications of wristed apparatus that offer multiple motions (or DoF) and that are controlled by a human user, there are often competing requirements.

The DoF or motions offered by the apparatus have to be controllable by the user such that the muscle groups engaged by the user to drive these DoF (at the input of the apparatus) do not get overly taxed or strained. Otherwise, this can lead to discomfort and fatigue for the user.

At the same time, in many applications such as surgery or minimally invasive surgery, it is important that the user is able to control the various DoF of the wristed apparatus in a very precise, repeatable, and controllable manner. This requires fine and sensitive control over the quantity and quality of motion of the distal manipulator.

Furthermore, it is desirable for the user to receive haptic feedback via the device so that they are able to sense or gauge the interaction (e.g. force, torque, pressure, relative displacement) between the distal manipulator of the wristed apparatus and the external object that is being manipulated by the distal manipulator and controlled by the user. In some applications like surgery or minimally invasive surgery, this haptic feedback can provide critical information, not easily gauged visually or by other means, back to the user (e.g. surgeon). Lack of such information can prove to be risky for the patient being operated on.

Finally, in applications of the wristed apparatus being held and operated by the human user, the apparatus has to be relatively compact in size and weight so that it does not become burdensome or tiresome for the user to handle and use over a period of time.

The above requirements of user comfort in driving the DoF of the wristed apparatus, fine and precise control of the DoF at the distal manipulator, haptic feedback from the distal manipulator back to the user, and compact size and weight of the apparatus are applicable in a wide range of applications beyond surgery. These applications may include precision fabrication and/or assembly, delicate material handling, or remote exploration in settings where access is restricted.

However, it is difficult to meet all of the above requirements in a given design of wristed apparatus because many of these requirements compete against each other.

For example, one approach to achieve good haptic feedback in a wristed apparatus is to use mechanical transmission between the user and all the way to the distal manipulator. Such a mechanical transmission helps provide a physical path not only for the user's inputs to be transmitted via the apparatus to the distal manipulator, but also provides the same path for forces (including loads, torques, etc.) at the distal manipulator to flow back to the user. The resulting “feel” of forces being applied by and at the distal manipulator can be invaluable for the user.

But a purely mechanical transmission can be taxing on the muscles engaged by the user to drive the various DoF of the wristed apparatus. A mechanical transmission requires that the user has to generate enough driving forces at the input to not only produce the desired forces at the distal manipulator, but also overcome the inertia and frictional resistance/losses associated with the mechanical transmission. This can be a problem particularly for user inputs that require muscle groups associated with fine motor control (e.g. fingers). In particular, if the user's fingers are used to drive certain DoF of the wristed apparatus, e.g. turning the roll dial, then the muscle groups associated with finger movement can easily get strained and fatigued.

Thus, with a wristed apparatus that employs purely mechanical transmission, while the user gains haptic feedback, they are also prone to discomfort and fatigue associated with fine motor control muscle groups.

To reduce the burden on the user's muscle groups that are engaged in driving the various DoF of the wristed apparatus, an alternative approach is to use mechatronic (or electronic) transmission to control all the DoF a wristed apparatus. Mechatronic transmission here implies that the user interface on the wristed apparatus for any given DoF may comprise a sensor that picks up the user intent or command for that DoF/motion. This command is captured via an electronic signal that is sent to a microcontroller, which in turn runs a control algorithm/logic and that sends a suitable amount of power (typically via a driver) to an actuator (e.g. an electric motor) within the wristed apparatus. Via the actuator and various mechanical transmission elements, the necessary driving forces for the DoF are delivered from the actuator to the distal manipulator. Given the combination of mechanical, electronic, and electromechanical components involved, this transmission may be referred to as a “mechatronic” transmission.

A key advantage of using mechatronic transmission for all the DoF in a wristed apparatus is that the various muscle groups (of the user) will experience lower burden in terms of the amount of input forces and torques needed to drive the multiple DoFs. Much of the forces, torques, and power needed at the distal manipulator are drawn from the actuators and not the user. Also, the motorized power and microprocessor control of the various DoF can provide fine and precise control of DoFs of the distal manipulator by the user.

But on the flip side, since a direct physical or mechanical transmission from the user to the DoFs of the distal manipulator no longer exists, haptic or force feedback from the distal manipulator back to the user's hand is also compromised. As noted previously, haptic feedback can be crucial for precision manipulation applications.

Furthermore, in this architecture of the wristed apparatus where all the DoF use a mechatronic transmission, it is necessary to have actuators (e.g. electric motors) and associated drivers, electronics, power sources (e.g. battery), microcontroller capabilities, etc. for each of the DoF. This increases the cost and complexity of the design, and makes the overall physical design more bulky and heavy. This added size and weight, in turn, makes it more difficult, burdensome, and tiresome for the user to handle and operate the wristed apparatus.

Thus, there are clear tradeoffs between the two architectures for wristed apparatus—one, where all the DoF are driven purely mechanically, and second, where all the DoF are motorized and are driven mechatronically.

These tradeoffs may be overcome by employing a hybrid architecture for a wristed apparatus, where some DoF may employ a mechanical transmission, while other DoF employ a mechatronic transmission from the user to the distal manipulator. In particular, DoF that require the use of fine motor muscles (e.g. in fingers) may employ mechatronic transmission. This can relieve strain and fatigue on those smaller muscle groups of the user while providing fine and precise movements at the distal manipulator. At the same time, DoFs that engage major muscle groups (e.g. in the forearm to articulate the wrist) may not need motorized power (e.g. from an electric motor), and can maintain a mechanical transmission. Since large muscle groups are less prone to fatigue, precise control of these DoF is maintained. At the same time, mechanical transmission ensures haptic feedback. And, the fact that fewer DoF employ a mechatronic transmission implies fewer actuators (e.g. motors), drivers, battery capacity, etc. This hybrid architecture makes the overall wristed apparatus more compact and lighter, compared to an architecture where all the DoF are motorized.

In particular, the preferred embodiment of the wristed apparatus 2000 shown here includes motorized power and mechatronic transmission for the Roll rotation of the distal manipulator. Roll control via a dial requires inputs from the user's fingers, which engage small muscle groups that are most prone to fatigue. Motorized power and microcontroller control overcomes this challenge. However, mechanical transmission is used for driving the Yaw and Pitch rotations and implicitly provides haptic feedback. Since these are driven by the user's wrist that engages large muscle groups in the forearm, muscle fatigue is less of a concern. Overall, this leads to a more compact and lighter overall apparatus.

In applications such as surgery or minimally invasive surgery of a wristed apparatus handled by a user, motorizing the roll rotation helps achieve the fine and precise motion required for suturing without fatiguing the associated muscles and losing fine motor control. Simultaneously, by ensuring mechanical transmission of the Yaw and Pitch rotations, the user receives valuable haptic feedback that is critical in surgical applications. This haptic feedback can be used by the user, such that they can adjust their inputs, without the need for visual feedback, in order to achieve more favorable interaction between the distal manipulator and the object that it is being manipulated (e.g. soft tissue of a patient). This haptic feedback can provide life saving information to the user, and this information cannot be perceived visually or by any other means.

While we have shown mechatronic transmission for Roll DoF, in principle a similar mechatronic transmission could be used for the Pitch and/or Roll DoF as well, within the overall framework of input articulation joint that wraps around a user's hand and the swashbox assembly presented herein.

We want to make sure that Pitch and Yaw are defined generally as two orthogonal rotations that define articulation. The specific axes within the plane of articulation do not matter. What is shown in the figures and the embodiments are simply representative.

While electric motors have been mentioned as a potential actuator to use for the mechatronic transmission for the Roll DoF, other rotary and linear actuators, with suitable transmissions, may be considered. Rotary actuators can include electric, electromagnetic, air motor/turbine, fluidic motor/turbine, etc. Linear actuators can include electric and electromagnetic motors, voice coils, piezoelectric, electrostatic, and fluidic piston-cylinders to name a few. Transmission systems include various types of gears (spur, helical, bevel, planetary, etc.), belts, cables, chains, linkages, flexures, etc.

The wristed apparatus 2000 shown here may be used in various surgical applications, in particular minimally invasive where dexterity and precision are critical in difficult to reach spaces within the patient body. Such needs arise in various surgical specialties, including but not limited to laparoscopy (i.e. minimally invasive surgery in the abdominal space), cardiothoracic surgery, vascular surgery, orthopedic and spine surgery, cranial and facial surgery, ENT surgery, ophthalmic surgery, and neurosurgery, to name a few.

Depending on the surgical specialty, certain sizes and shapes may have to be changed to suit the anatomy and workspace. For example, the Distal Manipulator instruments and size; the Handle assembly size and shape; length, diameter and shape of the tool shaft; nominal location of Handle body with respect to the chassis; size and shape of the chassis, etc. may be modified as needed, while retaining the core operating principles of providing Yaw, Pitch, and Roll rotations at a Distal Manipulator.

When used in laparoscopic surgery (i.e. minimally invasive surgery in the abdominal space), the Distal Manipulator can take various different shapes, size, form, and features to enable various different functions such as: holding a needle to drive it via tissue during suturing, holding a suture for knot tying; grasping; dissection; shearing/cutting; electrocauterization.

The Distal Manipulator could be equipped with jaws that open and close. Typically, there may be two jaws but in other instances, there could be three or more jaws as well.

In the case of two jaws at the Distal Manipulator, in some instances only one jaw may move relative to the other, while in other instances both jaws may move relative to each other.

In some instances, the Distal Manipulator may not have two or more jaws (or moving members) and instead only have a single member that is suitably shaped such as a hook cautery or spatula.

Accordingly, the Distal Manipulator of the wristed apparatus could be any of the following instrument types: Needle Driver/Holder, Traumatic Grasper, Atraumatic Grasper, Dissector, Shears, Hook Cautery, Monopolar or Bipolar Electrocautery. Other instrument types may include surgical staplers (linear or circular) and vessel sealers that use advanced energy (e.g. ultrasonic).

When used in cardiothoracic or vascular surgery, the distal manipular could comprise forceps (e.g. Resano toothing forceps, suture forceps, knot pusher forceps, diathermy forceps, etc.), needle holders (e.g. ryder needle holder, mini-jaw needle holder, coronary needle holder, locking and non-locking needle holders, etc.).

Other instrument types at the Distal Manipulator for vascular surgery could include Pott-Diethrich valve scissors for dissecting vessels and enlarge vascular incisions, and nerve and vessel hooks used to manipulate and probe valves or veins.

Instrument types at the Distal Manipulator of the wristed apparatus for cardiac surgery could include Retractors for holding incisions open and holding back tissues to maintain a clean surgical field (e.g. swiveling blade—straight and curved, Fixed blades, rultract, sternal, trans-thoracic wall retractor, etc.); sternal saw used for median sternotomy, opening the patient's chest by splitting the breastbone to access the heart and lungs; Rumel tourniquet passer to tighten purse string sutures to control bleeding at cannulation site; Bulldog appliers used to grip and hold tissues, vessels, or sutures; and cardiovascular clamps (e.g. Derra partial occlusion, Debakey, aortic cross, etc.) used to temporarily clamp blood vessels for hemostasis.

Instrument types at the Distal Manipulator of the wristed apparatus adapted for use in neurosurgery may include curettes (e.g. Spinal Fusion, Scoop) that are used to extract tissue samples from bones; Dissectors (e.g. Double-ended, dural guide and director, Penfield, Probe, etc.); Elevators (e.g. spinal, periosteal, dura, etc.) that are used to separate hard tissue around the bones to expose them for surgery; Forceps (e.g. Dressing, sweet clip applying, spatula, iris, etc.); Hooks (e.g. Adson hook, Adson oliver neuro, Cairns dural, Meninges, Oliver dural); Retractors; Rongeurs (e.g. Bateman, Cairns, Dandys, Daniel, Love-Grunwald, Northfield); and Scissors (e.g. Cairns, Olivecrona, Pituitary, Schmieden dural, etc.).

Instrument types at the Distal Manipulator of the wristed apparatus adapted for use in Orthopedic and Spine Surgery include Rongeurs (e.g. thin footplate, kerrison, ceramic bone injecting, ceramic open up, gerard, ferris smith, IVD, adons, leksell) used to gouge out bone or remove small pieces of tissue; Retractors (e.g. lateral plif, zelpi with cerebellar, offset zelpi, wiltse gelpi, miskimmon, adson cerebellar, meyerding) used to keep an incision open, hold back tissues and organs, or reach other structures; Curettes (e.g. long, lateral angle, cone ring, O' Brien, Charnley, triangle endplate, teardrop, down pushing, toothed long, american, pedicle, american half, micro half, american ring, american rainbow, micro rainbow, micro, micro axial) used to scrape or remove tissue, debris or foreign substances during surgery; Elevators (e.g. long handle cobb, long cobb, shovel nose, flat cobb, angled cobb, endplate scraper, woodson, long penfield, curved chisel, neidre, fellrath PLL, impactors, freer, key periosteal, anulus cutting, axial woodson, watson chain, howorth) used to elevate, scrape, or dissect bones, tissues, and nerves during a variety of surgical procedures; Rasps (e.g. mini, foraminal, single-sided endplate, lateral bayonet, double sided endplate, double sided, double sided lateral angle) that are used to sculpt bone in surgical procedures; Soft tissue screw retractors that fit around a pedicle screw; Gouges (smith perterson, piggot, ferret, harvesting, hibbs) used for cutting or removing bone during surgery; and Osteotomes (lambotte, thin shaft) used for cutting or preparing bones.

Instrument types at the Distal Manipulator of the wristed apparatus adapted for use in Ophthalmic Surgery include cannulas that may be used to irrigate and/or aspirate the interior of the eye during the surgical treatment or for Air Injection, Anesthesia, Aspirating, Backflush, Hydrodissection, etc. Other instrument types may include choppers (e.g. Femtosecond, nucleus, phaco) used to facilitate the manipulation of lens nucleus or other intraocular structures or sutures. Other instrument types at the Distal Manipulator of the wristed apparatus adapted for use in Ophthalmic Surgery may include: Forceps, Manipulator, Needle Holders, Markers, Retractors, Probes, Scissors, Speculum, Trephines, Spatulas, Calipers, etc.

The Device (FIG. 32) in the Wristed Apparatus (FIG. 20A) could be operated by a user holding the handle assembly 400 with one hand, while the shaft 241 could be supported externally for example by a trocar, in case of minimally invasive surgery.

The shaft 241 or the swashbox assembly 2200 or the chassis subassembly 310 could be supported by the user's other hand, or could be supported via an external support structure e.g. frame, or table mount, tripod, robotic arm, etc. such that the user does not have to bear the weight of the apparatus but can drive and control the inputs of the apparatus.

The wristed apparatus shown here can be used for various industrial style tasks—remote access tasks, assembly, pick and place, complex repair in difficult to reach spaces, precision fabrication, 3D printing, delicate material handling, or remote exploration when access is restricted, etc. Various types of tools or instruments may be equipped at the distal manipulator such as graspers, welding tips, drill bits, metal cutting tools etc.

The use of a cable transmissions to transmit motion from the Handle Assembly 400 is one particular and preferred embodiment of the Input Linkage Assembly 300. Alternatively, a series of linkage mechanisms can be used instead of or in addition to the cable transmission to transmit the motions of the Handle Assembly 400 (particularly yaw and pitch rotations) to the Swashbox Assembly 2200.

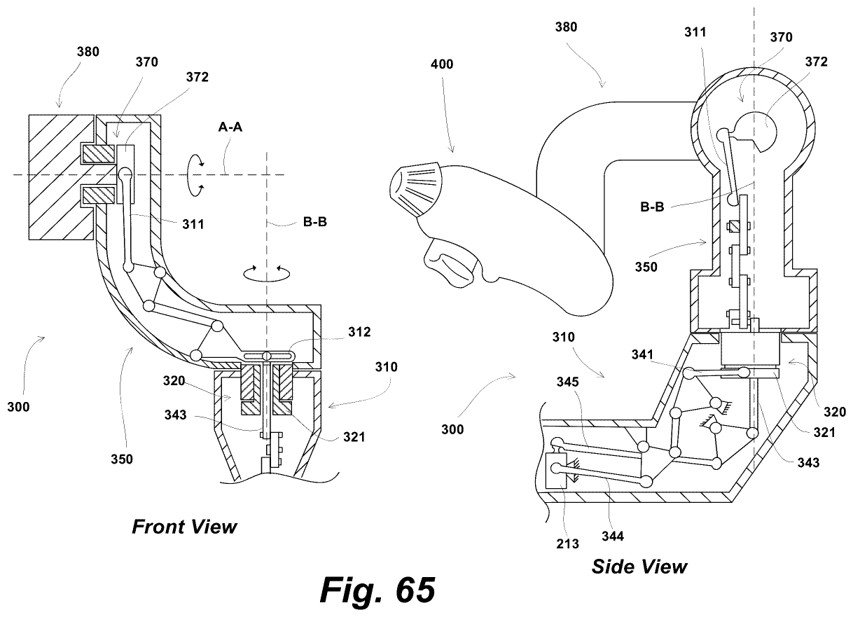

FIG. 65 depicts two section views, a Front View and a Side View, of an alternative Input Linkage Assembly 300 that uses linkages to transmit the yaw and pitch rotations of the Handle Assembly 400 to the Non-Rotating Plate 213.

Referring to FIG. 65, this particular embodiment transmits the rotation of the Pitch Driving Pulley 372 to a Drive Link 311 with two spherical joints on each end. This Drive Link 311 converts the rotational motion of the Pitch Driving Pulley 372 into linear motion and actuates the links connected to it throughout the Yaw Link Assembly 350. The rotation of the Yaw Link Subassembly 350 about Axis B-B with respect to the Chassis Subassembly 310 is decoupled from this linkage as it actuates adjacent links in the Chassis Subassembly 310 by keeping the Push Link 343 centered on Axis B-B by using a Slider 312. The Slider 312 will allow the Push Link 343 to be constrained along Axis B-B such that the Push Link 343 moves upward along Axis B-B as the Slider 312 rotates counterclockwise (in the Front View of FIG. 65) when the linkage is actuated. The pin on the Push Link 343 will be allowed to rotate with the Yaw Link Assembly 350 and the Slider 312 about Axis B-B by the use of a revolute joint aligned with Axis B-B. Therefore, only the upward and downward motion of the Push Link 343 will be transmitted through the remaining linkage in the Chassis Assembly 310 to the Output Link 345 which drives the Non-Rotating Plate 213.

The Yaw Transmission works in a very similar way. Shown in FIG. 65, the Yaw Driving Pulley 321 actuates the Drive Link 341 very similar to how the Pitch Driving Pulley 372 drives its Drive Link 311. The Drive Link 341 drives the rest of the linkage up to Output Link 344 which actuates the Non-Rotating Plate 213.

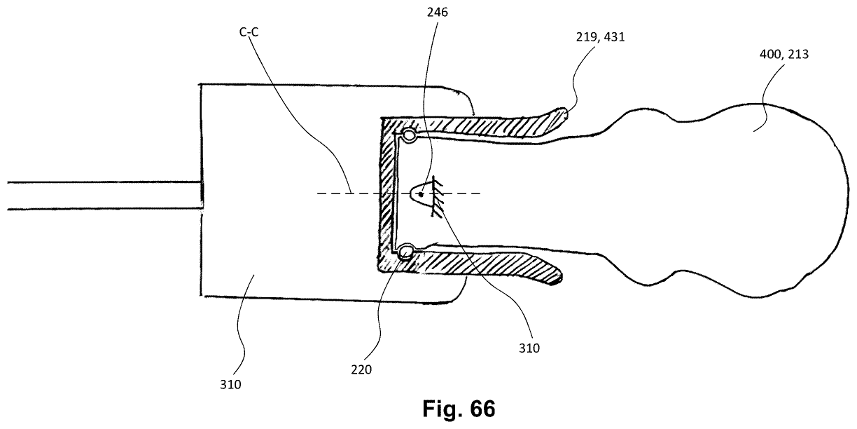

In another alternative architecture of the device, the transmission of motion from the handle assembly 400 to the non-rotating plate 213 can be via a rigid connection. FIG. 66. depicts a kinematic diagram of a possible embodiment. The handle assembly 400 and non-rotating plate 213 form a monolith (or are rigidly connected to one another), which is connected to the chassis subassembly 310 via a gimbal (universal) joint 246. Note that the axes of rotation of the gimbal joint 246 are orthogonal to the axis C-C. The dial 431 and rotating plate 219 form a monolith (or are rigidly connected to one another), which is coupled to the handle assembly 400/non-rotating plate 213 monolith via the rotating plate bearing 220, such that any rotation of the handle assembly 400/non-rotating plate 213 monolith with respect to the chassis subassembly 310 as enabled by the gimbal (universal) joint 246 is inherited by the dial 431/rotating plate 219 monolith. Rotation of the dial 431 with respect to the handle assembly 400/non-rotating plate 213 monolith is also transmitted to the rotating plate 219 via rigid connection. The dial 431 and rotating plate 219 form a monolith which can rotate about axis C-C with respect to the chassis subassembly 310 and the handle assembly 400/non-rotating plate 213 monolith due to the rotating plate bearing 220.

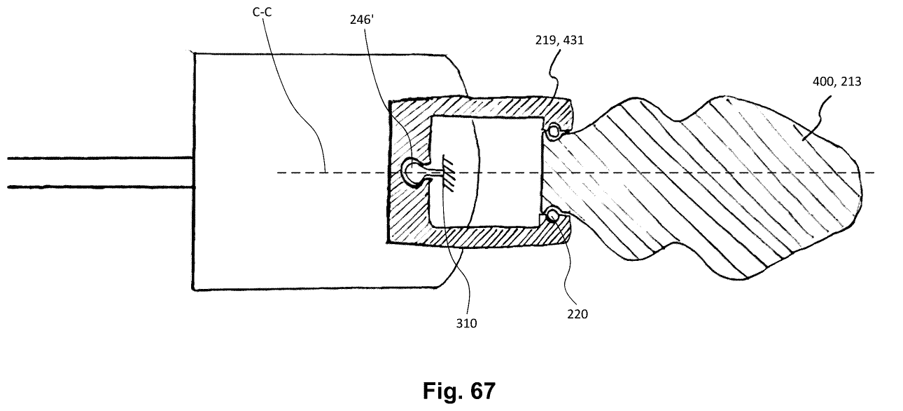

FIG. 67 depicts a kinematic diagram of another possible embodiment of the aforementioned rigid connection transmission architecture. The dial 431 and rotating plate 219 form a monolith (or are rigidly connected to one another) which is connected to the chassis subassembly 310 via a spherical joint 246′. The handle assembly 400 and non-rotating plate 213 form a monolith (or are rigidly connected to one another), which is coupled to the dial 431/rotating plate 219 monolith via the rotating plate bearing 220, such that any rotation (with the exception of rotation about the axis C-C) of the handle assembly 400/non-rotating plate 213 monolith with respect to the chassis subassembly 310, as enabled by the spherical joint 246′, is inherited by the dial 431/rotating plate 219 monolith. Rotation of the handle assembly 400/non-rotating plate 213 monolith about axis C-C is not inherited by the dial 431/rotating plate 219 monolith. The rotation of the dial 431/rotating plate 219 monolith about axis C-C with respect to the chassis subassembly 310 is enabled by the spherical joint 246′.

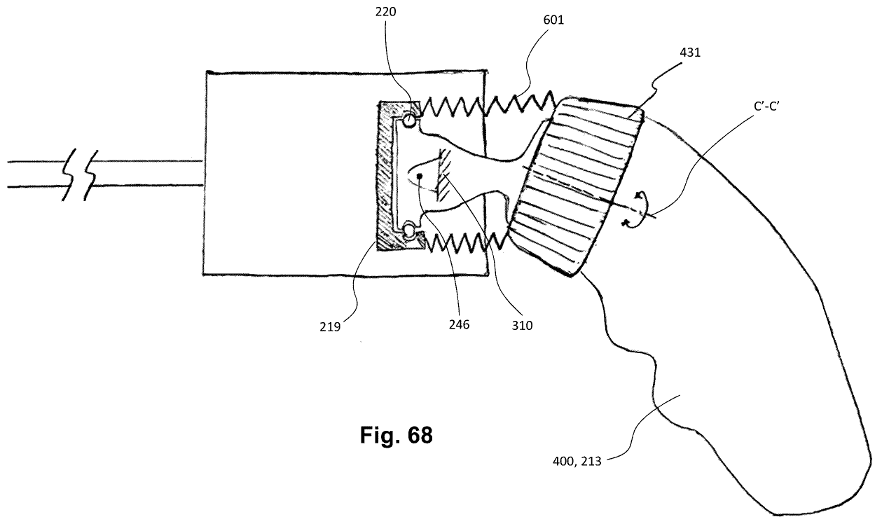

FIG. 68. depicts a kinematic diagram of another possible embodiment of the aforementioned rigid connection transmission architecture. The handle assembly 400 and non-rotating plate 213 form a monolith (or are rigidly connected to one another), which is connected to the chassis subassembly 310 via a gimbal (universal) joint 246. The rotating plate 219 is coupled to the handle assembly 400/non-rotating plate 213 monolith via the rotating plate bearing 220, such that any rotation of the handle assembly 400/non-rotating plate 213 monolith with respect to the chassis subassembly 310 as enabled by the gimbal (universal) joint 246 is inherited by the rotating plate 219. Rotation of the dial 431 with respect to the handle assembly 400/non-rotating plate 213 monolith about axis C′-C′ is transmitted to the rotating plate 219 via bellows 601 as depicted, although alternate coupling mechanisms are also possible, such as linkages, flexures etc.

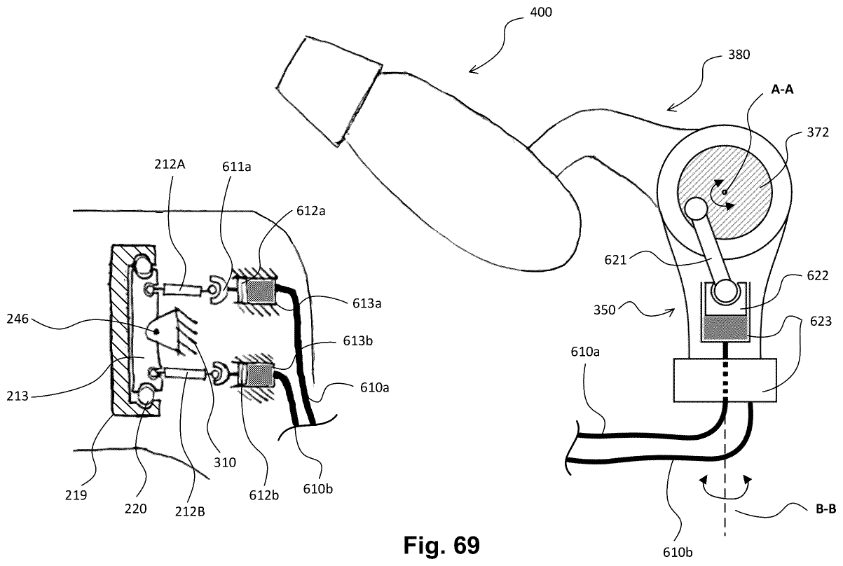

In yet another alternative architecture of the device, the transmission of motion from the handle assembly 400 to the non-rotating plate 213 can be via fluid transmission, where the fluid can be either hydraulic or pneumatic. FIG. 69 depicts a possible embodiment of such architecture.

Referring to FIG. 69, the handle assembly 400 is rigidly attached to the pitch link subassembly 380. Rotation of the handle assembly 400 relative to yaw link subassembly 350 about axis A-A results in the rotation of the pitch driving pulley 372, which displaces a connecting rod 621 that is attached to the pitch driving pulley 372 on one end via a spherical joint. The other end of the connecting rod 621 is connected to a driving piston 622 via another spherical joint. The driving piston 622 is allowed to translate within a cylinder 623 containing fluid, which is rigidly connected to the yaw link subassembly 350. Note that the two spherical joints of the connecting rod 621 can also possibly be revolute joints or universal joints. Hence, Rotation of the handle assembly 400 relative to the yaw link subassembly 350 about axis A-A results in the driving piston 622 compressing/decompressing the fluid within cylinder 623. Note that cylinder 623 can be a linear cylinder or a rotary cylinder. Referring to FIG. 69, a rotary type cylinder 623 is used to transmit yaw rotation.

Compression and decompression of the fluid is transmitted via a fluid line 610a to the swashbox assembly 2200, where there is a cylinder 613a with a pitch driven piston 612a that will be displaced along the cylinder in response to the changing fluid pressure in fluid line 610a. The pitch driven piston 612a is coupled to the pitch driven link 2212a via a spherical joint 611a, such that any displacement of the pitch driven piston 612a will be transmitted to the pitch driven link 2212a. The pitch driven link 2212a is then coupled to the nonrotating plate 213 via another spherical joint. The nonrotating plate is connected to the chassis subassembly 310 via a gimbal (universal joint) 246, such that displacement of the pitch driven link 2212a approximately along its longitudinal axis results in rotation of the nonrotating plate 213 about the gimbal (universal) joint 246. Therefore, rotation of the handle assembly 400 and pitch link sub assembly 380 relative to the yaw link subassembly about axis A-A will result in the tip/tilt of the nonrotating plate 213 about gimbal (universal) joint 246 relative to the chassis assembly 310. The rotating plate 219 is allowed to rotate relative to the non rotating plate 213 via the rotating plate bearing 220.

The rotation of the yaw link assembly 350 relative to the chassis assembly 310 about axis B-B can also be transmitted via a similar mechanism described above. Compression/decompression of the fluid 624 is transmitted via fluid line 610b to the swashbox assembly 2200, where a yaw driven piston 612b is displaced along cylinder 613b, which actuates the yaw driven link 212b resulting in the tip/tilt of the nonrotating plate 213 about gimbal (universal) joint 246 relative to the chassis assembly 310.

The yaw driven link 212b and pitch driven link 212a shown in FIG. 69 are attached to the non-rotating plate 213 in such a way that each mechanism actuates the non-rotating plate 213 relative to chassis 310 via the gimbal joint 246 about an axis that is orthogonal to the other.

In this particular architecture, rotation of the handle assembly 400 relative to yaw link subassembly 350 about axis A-A will only actuate the pitch driven link 212a, and rotation of the yaw link assembly 350 relative to the chassis assembly 310 about axis B-B will only actuate the yaw driven link 212b, because the two corresponding fluid lines are independent, such that actuating one of the fluid lines will not affect the pressure of the other fluid line.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

For purposes of this disclosure, the term “coupled” (in all of its forms, couple, coupling, coupled, etc.) generally means the joining of two components directly or indirectly to one another. Such joining may be stationary in nature or movable in nature; may be achieved with the two components and any additional intermediate members being integrally formed as a single unitary body with one another or with the two components; and may be permanent in nature or may be removable or releasable in nature, unless otherwise stated.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional implementations that also incorporate the recited features. Furthermore, the terms “first,” “second,” and the like, as used herein do not denote any order, quantity, or importance, but rather are used to denote element from another.

Numbers, percentages, ratios, or other values stated herein are intended to include that value, and also other values that are “about” or “approximately” the stated value, as would be appreciated by one of ordinary skill in the art encompassed by implementations of the present disclosure. A stated value should therefore be interpreted broadly enough to encompass values that are at least close enough to the stated value to perform a desired function or achieve a desired result. For example, the terms “approximately,” “about,” and “substantially” may refer to an amount that is within less than 5% of, within less than 1% of, within less than 0.1% of, and within less than 0.01% of a stated amount.

Further, it should be understood that any directions or reference frames in the preceding description are merely relative directions or movements. For example, the terms “upper,” “lower,” “right,” “left,” “rear,” “front,” “vertical,” “horizontal,” “inboard,” “outboard” and derivatives thereof shall relate to the orientation shown in FIG. 1. However, it is to be understood that various alternative orientations may be provided, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Changes and modifications in the specifically described embodiments may be carried out without departing from the principles of the present invention, which is intended to be limited only by the scope of the appended claims as interpreted according to the principles of patent law. The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

What is claimed is:

1. A surgical device comprising:
a distal manipulator movable between a nominal orientation and articulated orientations and rotatable in the articulated orientations with unlimited range of motion;
a handle movable by a hand of a user between a second nominal orientation and second articulated orientations according to which the distal manipulator is moved between the nominal orientation and the articulated orientations and a roll input for receiving user input to rotate the distal manipulator in the articulated orientations;
a roll transmission that causes the distal manipulator to rotate according to the user input; and
an articulation transmission that maintains the distal manipulator in the articulated orientations as the distal manipulator is rotated by the roll transmission, the articulation transmission having an output member that is movable between a third nominal orientation and third articulated orientations and rotatable in the third articulated orientations with unlimited range of motion.

2. The surgical device according to claim 1, wherein the second nominal orientation corresponds to the nominal orientation and the third nominal orientation, and the second articulated orientations correspond to the articulated orientations and the third articulated orientations.

3. The surgical device according to claim 1, wherein movement of the handle assembly in pitch and yaw directions causes the output member and the distal manipulator to move in pitch and yaw directions.

4. The surgical device according to claim 3, wherein movement of the handle assembly in pitch and yaw directions relative to a frame of reference causes the output member and the distal manipulator to move in pitch and yaw directions relative to the frame of reference.

5. The surgical device according to claim 1, further comprising a shaft;
wherein the distal manipulator is pivotably coupled to the shaft; and
wherein the roll transmission includes a motor that rotates the shaft according to the user input.

6. The surgical device according to claim 5, wherein the shaft transfers torque from the motor of the roll transmission to the distal manipulator to cause rotation thereof in the articulated orientations.

7. The surgical device according to claim 5, wherein the shaft is coupled to the output member and transfers torque thereto to cause rotation thereof, and wherein the shaft receives torque from the electric motor and transfers torque to the distal manipulator and the first structure in parallel to cause rotation thereof.

8. The surgical device according to claim 1, wherein the roll input is a roll dial that is coupled to a distal end of the handle away from the user and rotatable relative thereto for receiving the user input.

9. The surgical device according to claim 1, further comprising an input transmission having a first pulley and a first drive link that is coupled to and extends between the first pulley and an input member of the articulation transmission;
wherein the articulation transmission includes the input member coupled to the output member, the output member being rotatable there relative to the input member t an axis and constrained in all other movement relative thereto; and
wherein when the first pulley is rotated by the handle, the first drive link pushes and pulls the input member to move the output member between the third nominal orientation and the third articulated orientations.

10. The surgical device according to claim 9, wherein input transmission includes a second pulley and a second drive link that is coupled to and extends between the first pulley and the input member;
wherein the when second pulley is rotated by the handle, the second drive link pushes and pulls the input member to move the output member between the third nominal orientation and the third articulated orientations;
wherein the first drive link and the second drive link are coupled to the input member at positions spaced apart by approximately 90 degrees about a longitudinal axis of the input member;
wherein the first pulley and the second pulley rotate about axes that are perpendicular to each other;
wherein the input transmission includes cable lengths that are pulled by the handle to rotate the first pulley and the second pulley to move the output member;
wherein the handle includes a first link and a second link, the first link being rotatably coupled at a first pivot joint to the second link, and the second link 522b being rotatably coupled at a second pivot joint to a chassis that contains the articulation transmission; and
wherein first cable lengths of the cable lengths extend from another first pulley at the first pivot joint to the first pulley, and second cable lengths of the cable lengths extend from another second pulley at the second pivot joint through the first pivot joint to the second pulley.

11. A device for manipulating an object comprising:
a distal manipulator for engaging the object, the distal manipulator movable between a reference orientation and articulated orientations, and is rotatable in the articulated orientations with unlimited range of motion;
a roll transmission that causes the distal manipulator to rotate, the roll transmission having a motor;
an articulation transmission that maintains the distal manipulator in the articulated orientations as the distal manipulator is rotated by the roll transmission, the articulation transmission having an output member that is movable between another reference orientation and other articulated orientations that correspond to the reference orientation and the articulated orientations of the distal manipulator.

12. The device according to claim 11, further comprising a shaft;
wherein the distal manipulator is pivotably coupled to the shaft; and
wherein the shaft transfers torque from the motor of the roll transmission to the distal manipulator to cause rotation thereof.

13. The device according to claim 11, further comprising a shaft;
wherein the distal manipulator pivotably coupled to the shaft; and
wherein the shaft is coupled to the output member and transfers torque thereto to cause rotation thereof.

14. The device according to claim 13, further comprising a linkage that transfers torque between the shaft and the output member.

15. The device according to claim 14, wherein the linkage includes a first end coupled to the shaft and a second end coupled to the output member, and the linkage accommodates changes in distance between first end and second end as the output member rotates while in the articulated orientations.

16. The device according to claim 15, wherein the shaft rotates about a longitudinal axis, the first end rotates at a fixed radial distance about the longitudinal axis, and the second end rotates at variable radial distances about the longitudinal axes as the output member rotates in the other articulated orientations; and wherein the shaft rotates about a longitudinal axis, the first end of the linkage is coupled to the shaft at a fixed longitudinal position along the axis, and the second end of the linkage varies in longitudinal position along the longitudinal axis as the output member rotates in the other articulated orientations.

17. The device according to claim 11, further comprising a shaft;
wherein the distal manipulator pivotably coupled to the shaft; and
wherein the shaft receives torque from the electric motor and transfers torque to the distal manipulator and the first structure in parallel to cause rotation thereof.

18. The device according to claim 11, further comprising a shaft;
wherein the distal manipulator pivotably coupled to the shaft; and
wherein the shaft rotates about a longitudinal axis, the distal manipulator rotates about the longitudinal axis when in the nominal orientation, and the output member rotates about the longitudinal axis in the other nominal orientation.

19. The device according to claim 12, further comprising cables having output cable lengths that extend through the shaft and are coupled to the output member and the distal manipulator, such that as the output member moved between the other nominal orientation and the other articulated orientations, the distal manipulator moves between the nominal orientation and the articulated orientations, respectively.

20. The device according to claim 11, further comprising a housing;
wherein the articulation transmission is a swashplate mechanism contained within the housing and includes the output member and an input member, the input member being pivotably supported within the housing to pivot about a first axis and a second axis that are perpendicular to each other and being constrained from in all other movement relative to the housing;
wherein the input member is pivoted according to articulation inputs, and the output member rotates relative to the input member and is constrained in all other movements relative to the input member;
wherein the output member is spaced apart from the housing so as to rotate in the unlimited range of motion relative to the housing;
wherein the output member is movable between the other nominal orientation and the articulated orientation without engaging the housing; and
wherein a device frame of reference is fixed relative to the housing, and the output member is pivotable relative to the housing about a pitch axis and a yaw axis of the frame of reference between the other nominal orientation and the other articulated orientations.
